SEQUENCE LISTING (156 sequences)

SEQ ID NO: 1 - protein, 40 aa, Homo sapiens
  MISC_FEATURE (1)..(1): Xaa is His or Ala
XXXXXFTXXXXXXRXXXAXXXYLXXXXXXXXXXXXXXXXX

SEQ ID NO: 2 - protein, 31 aa, Homo sapiens
HSDAVFTDQYTRLRKQVAAKKYLQSIKQKRY

SEQ ID NO: 3 - protein, 31 aa, Homo sapiens
HSDAVFTDQYTRLRKQMAAKKYLQSIKQKRY

SEQ ID NO: 4 - protein, 29 aa, Homo sapiens
HSDAVFTDQYTRLRKQVAAKKYLQSIKQK

SEQ ID NO: 5 - protein, 31 aa, Homo sapiens
HTEAVFTDQYTRLRKQVAAKKYLQSIKQKRY

SEQ ID NO: 6 - protein, 31 aa, Homo sapiens
HSDAVFTDQYTRLRKQLAVKKYLQDIKQGGT

SEQ ID NO: 7 - protein, 30 aa, Homo sapiens
HSDAVFTDQYTRLRKQMAAKKYLQSIKQKR

SEQ ID NO: 8 - protein, 31 aa, Homo sapiens
HSDAVFTDQYTRLRKQLAAKKYLQTIKQKRY

SEQ ID NO: 9 - protein, 31 aa, Homo sapiens
HSDAVFTDQYTRLRKQMAAKKYLQTIKQKRY

SEQ ID NO: 10 - protein, 31 aa, Homo sapiens
HSDAVFTDQYTRLRKQMAAHKYLQSIKQKRY

SEQ ID NO: 11 - protein, 31 aa, Homo sapiens
HSDAVFTDQYTRLRKQMAAKHYLQSIKQKRY

SEQ ID NO: 12 - protein, 30 aa, Homo sapiens
HSDAVFTDQYTRLRKQMAGKKYLQSIKQKR

SEQ ID NO: 13 - protein, 30 aa, Homo sapiens
HSDAVFTDQYTRLRKQMAKKKYLQSIKQKR

SEQ ID NO: 14 - protein, 30 aa, Homo sapiens
HSDAVFTDQYTRLRKQMARKKYLQSIKQKR

SEQ ID NO: 15 - protein, 30 aa, Homo sapiens
HSDAVFTDQYTRLRKQMASKKYLQSIKQKR

SEQ ID NO: 16 - protein, 30 aa, Homo sapiens
HSDAVFTDQYTRLRKQMAAKKYLQSIPQKR

SEQ ID NO: 17 - protein, 30 aa, Homo sapiens
HSDAVFTDQYTRLRKQMAAKKYLQSIQQKR

SEQ ID NO: 18 - protein, 30 aa, Homo sapiens
HSDAVFTDQYTRLRKQMAAKKYLQSIRQKR

SEQ ID NO: 19 - protein, 30 aa, Homo sapiens
HSDAVFTDQYTRLRKQMAAKKYLQSIKQRR

SEQ ID NO: 20 - protein, 30 aa, Homo sapiens
HSDAVFTDQYTRLRKQMAAKKYLQSIKQKA

SEQ ID NO: 21 - protein, 30 aa, Homo sapiens
HSDAVFTDQYTRLRKQMAAKKYLQSIKQKF

SEQ ID NO: 22 - protein, 30 aa, Homo sapiens
HSDAVFTDQYTRLRKQMAAKKYLQSIKQKH

SEQ ID NO: 23 - protein, 30 aa, Homo sapiens
HSDAVFTDQYTRLRKQMAAKKYLQSIKQKI

SEQ ID NO: 24 - protein, 30 aa, Homo sapiens
HSDAVFTDQYTRLRKQMAAKKYLQSIKQKK

SEQ ID NO: 25 - protein, 30 aa, Homo sapiens
HSDAVFTDQYTRLRKQMAAKKYLQSIKQKL

SEQ ID NO: 26 - protein, 30 aa, Homo sapiens
HSDAVFTDQYTRLRKQMAAKKYLQSIKQKM

SEQ ID NO: 27 - protein, 30 aa, Homo sapiens
HSDAVFTDQYTRLRKQMAAKKYLQSIKQKP

SEQ ID NO: 28 - protein, 30 aa, Homo sapiens
HSDAVFTDQYTRLRKQMAAKKYLQSIKQKQ

SEQ ID NO: 29 - protein, 30 aa, Homo sapiens
HSDAVFTDQYTRLRKQMAAKKYLQSIKQKS

SEQ ID NO: 30 - protein, 30 aa, Homo sapiens
HSDAVFTDQYTRLRKQMAAKKYLQSIKQKT

SEQ ID NO: 31 - protein, 30 aa, Homo sapiens
HSDAVFTDQYTRLRKQMAAKKYLQSIKQKV

SEQ ID NO: 32 - protein, 30 aa, Homo sapiens
HSDAVFTDQYTRLRKQMAAKKYLQSIKQKW

SEQ ID NO: 33 - protein, 30 aa, Homo sapiens
HSDAVFTDQYTRLRKQMAAKKYLQSIKQKY

SEQ ID NO: 34 - protein, 30 aa, Homo sapiens
HSDAVFTDQYTRLRKQMAGKKYLQSIKQRI

SEQ ID NO: 35 - protein, 30 aa, Homo sapiens
HSDAVFTDQYTRLRKQMAKKKYLQSIKQRI

SEQ ID NO: 36 - protein, 30 aa, Homo sapiens
HSDAVFTDQYTRLRKQMASKKYLQSIKQRI

SEQ ID NO: 37 - protein, 30 aa, Homo sapiens
HSDAVFTDQYTRLRKQMAAKKYLQSIPQRI

SEQ ID NO: 38 - protein, 30 aa, Homo sapiens
HSDAVFTDQYTRLRKQMASKKYLQSIRQRI

SEQ ID NO: 39 - protein, 31 aa, Homo sapiens
HSDAVFTDNYTRLRKQVAAKKYLQSIKQKRY

SEQ ID NO: 40 - protein, 31 aa, Homo sapiens
HSDAVFTDNYTRLRKQMAAKKYLQSIKQKRY

SEQ ID NO: 41 - protein, 29 aa, Homo sapiens
HSDAVFTDNYTRLRKQVAAKKYLQSIKQK

SEQ ID NO: 42 - protein, 31 aa, Homo sapiens
HTEAVFTDNYTRLRKQVAAKKYLQSIKQKRY

SEQ ID NO: 43 - protein, 31 aa, Homo sapiens
HSDAVFTDNYTRLRKQLAVKKYLQDIKQGGT

SEQ ID NO: 44 - protein, 30 aa, Homo sapiens
HSDAVFTDNYTRLRKQMAAKKYLQSIKQKR

SEQ ID NO: 45 - protein, 31 aa, Homo sapiens
HSDAVFTDNYTRLRKQLAAKKYLQTIKQKRY

SEQ ID NO: 46 - protein, 31 aa, Homo sapiens
HSDAVFTDNYTRLRKQMAAKKYLQTIKQKRY

SEQ ID NO: 47 - protein, 31 aa, Homo sapiens
HSDAVFTDNYTRLRKQMAAHKYLQSIKQKRY

SEQ ID NO: 48 - protein, 31 aa, Homo sapiens
HSDAVFTDNYTRLRKQMAAKHYLQSIKQKRY

SEQ ID NO: 49 - protein, 30 aa, Homo sapiens
HSDAVFTDNYTRLRKQMAGKKYLQSIKQKR

SEQ ID NO: 50 - protein, 30 aa, Homo sapiens
HSDAVFTDNYTRLRKQMAKKKYLQSIKQKR

SEQ ID NO: 51 - protein, 30 aa, Homo sapiens
HSDAVFTDNYTRLRKQMARKKYLQSIKQKR

SEQ ID NO: 52 - protein, 30 aa, Homo sapiens
HSDAVFTDNYTRLRKQMASKKYLQSIKQKR

SEQ ID NO: 53 - protein, 30 aa, Homo sapiens
HSDAVFTDNYTRLRKQMAAKKYLQSIPQKR

SEQ ID NO: 54 - protein, 30 aa, Homo sapiens
HSDAVFTDNYTRLRKQMAAKKYLQSIQQKR

SEQ ID NO: 55 - protein, 30 aa, Homo sapiens
HSDAVFTDNYTRLRKQMAAKKYLQSIRQKR

SEQ ID NO: 56 - protein, 30 aa, Homo sapiens
HSDAVFTDNYTRLRKQMAAKKYLQSIKQRR

SEQ ID NO: 57 - protein, 30 aa, Homo sapiens
HSDAVFTDNYTRLRKQMAAKKYLQSIKQKA

SEQ ID NO: 58 - protein, 30 aa, Homo sapiens
HSDAVFTDNYTRLRKQMAAKKYLQSIKQKF

SEQ ID NO: 59 - protein, 30 aa, Homo sapiens
HSDAVFTDNYTRLRKQMAAKKYLQSIKQKH

SEQ ID NO: 60 - protein, 30 aa, Homo sapiens
HSDAVFTDNYTRLRKQMAAKKYLQSIKQKI

SEQ ID NO: 61 - protein, 30 aa, Homo sapiens
HSDAVFTDNYTRLRKQMAAKKYLQSIKQKK

SEQ ID NO: 62 - protein, 30 aa, Homo sapiens
HSDAVFTDNYTRLRKQMAAKKYLQSIKQKL

SEQ ID NO: 63 - protein, 30 aa, Homo sapiens
HSDAVFTDNYTRLRKQMAAKKYLQSIKQKM

SEQ ID NO: 64 - protein, 30 aa, Homo sapiens
HSDAVFTDNYTRLRKQMAAKKYLQSIKQKP

SEQ ID NO: 65 - protein, 30 aa, Homo sapiens
HSDAVFTDNYTRLRKQMAAKKYLQSIKQKQ

SEQ ID NO: 66 - protein, 30 aa, Homo sapiens
HSDAVFTDNYTRLRKQMAAKKYLQSIKQKS

SEQ ID NO: 67 - protein, 30 aa, Homo sapiens
HSDAVFTDNYTRLRKQMAAKKYLQSIKQKT

SEQ ID NO: 68 - protein, 30 aa, Homo sapiens
HSDAVFTDNYTRLRKQMAAKKYLQSIKQKV

SEQ ID NO: 69 - protein, 30 aa, Homo sapiens
HSDAVFTDNYTRLRKQMAAKKYLQSIKQKW

SEQ ID NO: 70 - protein, 30 aa, Homo sapiens
HSDAVFTDNYTRLRKQMAAKKYLQSIKQKY

SEQ ID NO: 71 - protein, 30 aa, Homo sapiens
HSDAVFTDNYTRLRKQMAGKKYLQSIKQRI

SEQ ID NO: 72 - protein, 30 aa, Homo sapiens
HSDAVFTDNYTRLRKQMAKKKYLQSIKQRI

SEQ ID NO: 73 - protein, 30 aa, Homo sapiens
HSDAVFTDNYTRLRKQMASKKYLQSIKQRI

SEQ ID NO: 74 - protein, 30 aa, Homo sapiens
HSDAVFTDNYTRLRKQMAAKKYLQSIPQRI

SEQ ID NO: 75 - protein, 30 aa, Homo sapiens
HSDAVFTDNYTRLRKQMASKKYLQSIRQRI

SEQ ID NO: 76 - protein, 31 aa, Homo sapiens
HSDAVFTDQYTRLRKQVAAKKYLQSIKNKRY

SEQ ID NO: 77 - protein, 31 aa, Homo sapiens
HSDAVFTDQYTRLRKQMAAKKYLQSIKNKRY

SEQ ID NO: 78 - protein, 29 aa, Homo sapiens
HSDAVFTDQYTRLRKQVAAKKYLQSIKNK

SEQ ID NO: 79 - protein, 31 aa, Homo sapiens
HTEAVFTDQYTRLRKQVAAKKYLQSIKNKRY

SEQ ID NO: 80 - protein, 31 aa, Homo sapiens
HSDAVFTDQYTRLRKQLAVKKYLQDIKNGGT

SEQ ID NO: 81 - protein, 30 aa, Homo sapiens
HSDAVFTDQYTRLRKQMAAKKYLQSIKNKR

SEQ ID NO: 82 - protein, 31 aa, Homo sapiens
HSDAVFTDQYTRLRKQLAAKKYLQTIKNKRY

SEQ ID NO: 83 - protein, 31 aa, Homo sapiens
HSDAVFTDQYTRLRKQMAAKKYLQTIKNKRY

SEQ ID NO: 84 - protein, 31 aa, Homo sapiens
HSDAVFTDQYTRLRKQMAAHKYLQSIKNKRY

SEQ ID NO: 85 - protein, 31 aa, Homo sapiens
HSDAVFTDQYTRLRKQMAAKHYLQSIKNKRY

SEQ ID NO: 86 - protein, 30 aa, Homo sapiens
HSDAVFTDQYTRLRKQMAGKKYLQSIKNKR

SEQ ID NO: 87 - protein, 30 aa, Homo sapiens
HSDAVFTDQYTRLRKQMAKKKYLQSIKNKR

SEQ ID NO: 88 - protein, 30 aa, Homo sapiens
HSDAVFTDQYTRLRKQMARKKYLQSIKNKR

SEQ ID NO: 89 - protein, 30 aa, Homo sapiens
HSDAVFTDQYTRLRKQMASKKYLQSIKNKR

SEQ ID NO: 90 - protein, 30 aa, Homo sapiens
HSDAVFTDQYTRLRKQMAAKKYLQSIPNKR

SEQ ID NO: 91 - protein, 30 aa, Homo sapiens
HSDAVFTDQYTRLRKQMAAKKYLQSIQNKR

SEQ ID NO: 92 - protein, 30 aa, Homo sapiens
HSDAVFTDQYTRLRKQMAAKKYLQSIRNKR

SEQ ID NO: 93 - protein, 30 aa, Homo sapiens
HSDAVFTDQYTRLRKQMAAKKYLQSIKNRR

SEQ ID NO: 94 - protein, 30 aa, Homo sapiens
HSDAVFTDQYTRLRKQMAAKKYLQSIKNKA

SEQ ID NO: 95 - protein, 30 aa, Homo sapiens
HSDAVFTDQYTRLRKQMAAKKYLQSIKNKF

SEQ ID NO: 96 - protein, 30 aa, Homo sapiens
HSDAVFTDQYTRLRKQMAAKKYLQSIKNKH

SEQ ID NO: 97 - protein, 30 aa, Homo sapiens
HSDAVFTDQYTRLRKQMAAKKYLQSIKNKI

SEQ ID NO: 98 - protein, 30 aa, Homo sapiens
HSDAVFTDQYTRLRKQMAAKKYLQSIKNKK

SEQ ID NO: 99 - protein, 30 aa, Homo sapiens
HSDAVFTDQYTRLRKQMAAKKYLQSIKNKL

SEQ ID NO: 100 - protein, 30 aa, Homo sapiens
HSDAVFTDQYTRLRKQMAAKKYLQSIKNKM

SEQ ID NO: 101 - protein, 30 aa, Homo sapiens
HSDAVFTDQYTRLRKQMAAKKYLQSIKNKP

SEQ ID NO: 102 - protein, 30 aa, Homo sapiens
HSDAVFTDQYTRLRKQMAAKKYLQSIKNKQ

SEQ ID NO: 103 - protein, 30 aa, Homo sapiens
HSDAVFTDQYTRLRKQMAAKKYLQSIKNKS

SEQ ID NO: 104 - protein, 30 aa, Homo sapiens
HSDAVFTDQYTRLRKQMAAKKYLQSIKNKT

SEQ ID NO: 105 - protein, 30 aa, Homo sapiens
HSDAVFTDQYTRLRKQMAAKKYLQSIKNKV

SEQ ID NO: 106 - protein, 30 aa, Homo sapiens
HSDAVFTDQYTRLRKQMAAKKYLQSIKNKW

SEQ ID NO: 107 - protein, 30 aa, Homo sapiens
HSDAVFTDQYTRLRKQMAAKKYLQSIKNKY

SEQ ID NO: 108 - protein, 30 aa, Homo sapiens
HSDAVFTDQYTRLRKQMAGKKYLQSIKNRI

SEQ ID NO: 109 - protein, 30 aa, Homo sapiens
HSDAVFTDQYTRLRKQMAKKKYLQSIKNRI

SEQ ID NO: 110 - protein, 30 aa, Homo sapiens
HSDAVFTDQYTRLRKQMASKKYLQSIKNRI

SEQ ID NO: 111 - protein, 30 aa, Homo sapiens
HSDAVFTDQYTRLRKQMAAKKYLQSIPNRI

SEQ ID NO: 112 - protein, 30 aa, Homo sapiens
HSDAVFTDQYTRLRKQMASKKYLQSIRNRI

SEQ ID NO: 113 - protein, 32 aa, Homo sapiens
HSDAVFTDQYTRLRKQVAAKKYLQSIKQKRYC

SEQ ID NO: 114 - protein, 32 aa, Homo sapiens
HSDAVFTDQYTRLRKQMAAKKYLQSIKQKRYC

SEQ ID NO: 115 - protein, 30 aa, Homo sapiens
HSDAVFTDQYTRLRKQVAAKKYLQSIKQKC

SEQ ID NO: 116 - protein, 32 aa, Homo sapiens
HTEAVFTDQYTRLRKQVAAKKYLQSIKQKRYC

SEQ ID NO: 117 - protein, 32 aa, Homo sapiens
HSDAVFTDQYTRLRKQLAVKKYLQDIKQGGTC

SEQ ID NO: 118 - protein, 31 aa, Homo sapiens
HSDAVFTDQYTRLRKQMAAKKYLQSIKQKRC

SEQ ID NO: 119 - protein, 32 aa, Homo sapiens
HSDAVFTDQYTRLRKQLAAKKYLQTIKQKRYC

SEQ ID NO: 120 - protein, 32 aa, Homo sapiens
HSDAVFTDQYTRLRKQMAAKKYLQTIKQKRYC

SEQ ID NO: 121 - protein, 32 aa, Homo sapiens
HSDAVFTDQYTRLRKQMAAHKYLQSIKQKRYC

SEQ ID NO: 122 - protein, 32 aa, Homo sapiens
HSDAVFTDQYTRLRKQMAAKHYLQSIKQKRYC

SEQ ID NO: 123 - protein, 31 aa, Homo sapiens
HSDAVFTDQYTRLRKQMAGKKYLQSIKQKRC

SEQ ID NO: 124 - protein, 31 aa, Homo sapiens
HSDAVFTDQYTRLRKQMAKKKYLQSIKQKRC

SEQ ID NO: 125 - protein, 31 aa, Homo sapiens
HSDAVFTDQYTRLRKQMARKKYLQSIKQKRC

SEQ ID NO: 126 - protein, 31 aa, Homo sapiens
HSDAVFTDQYTRLRKQMASKKYLQSIKQKRC

SEQ ID NO: 127 - protein, 31 aa, Homo sapiens
HSDAVFTDQYTRLRKQMAAKKYLQSIPQKRC

SEQ ID NO: 128 - protein, 31 aa, Homo sapiens
HSDAVFTDQYTRLRKQMAAKKYLQSIQQKRC

SEQ ID NO: 129 - protein, 31 aa, Homo sapiens
HSDAVFTDQYTRLRKQMAAKKYLQSIRQKRC

SEQ ID NO: 130 - protein, 31 aa, Homo sapiens
HSDAVFTDQYTRLRKQMAAKKYLQSIKQRRC

SEQ ID NO: 131 - protein, 31 aa, Homo sapiens
HSDAVFTDQYTRLRKQMAAKKYLQSIKQKAC

SEQ ID NO: 132 - protein, 31 aa, Homo sapiens
HSDAVFTDQYTRLRKQMAAKKYLQSIKQKFC

SEQ ID NO: 133 - protein, 31 aa, Homo sapiens
HSDAVFTDQYTRLRKQMAAKKYLQSIKQKHC

SEQ ID NO: 134 - protein, 31 aa, Homo sapiens
HSDAVFTDQYTRLRKQMAAKKYLQSIKQKIC

SEQ ID NO: 135 - protein, 31 aa, Homo sapiens
HSDAVFTDQYTRLRKQMAAKKYLQSIKQKKC

SEQ ID NO: 136 - protein, 31 aa, Homo sapiens
HSDAVFTDQYTRLRKQMAAKKYLQSIKQKLC

SEQ ID NO: 137 - protein, 31 aa, Homo sapiens
HSDAVFTDQYTRLRKQMAAKKYLQSIKQKMC

SEQ ID NO: 138 - protein, 31 aa, Homo sapiens
HSDAVFTDQYTRLRKQMAAKKYLQSIKQKPC

SEQ ID NO: 139 - protein, 31 aa, Homo sapiens
HSDAVFTDQYTRLRKQMAAKKYLQSIKQKQC

SEQ ID NO: 140 - protein, 31 aa, Homo sapiens
HSDAVFTDQYTRLRKQMAAKKYLQSIKQKSC

SEQ ID NO: 141 - protein, 31 aa, Homo sapiens
HSDAVFTDQYTRLRKQMAAKKYLQSIKQKTC

SEQ ID NO: 142 - protein, 31 aa, Homo sapiens
HSDAVFTDQYTRLRKQMAAKKYLQSIKQKVC

SEQ ID NO: 143 - protein, 31 aa, Homo sapiens
HSDAVFTDQYTRLRKQMAAKKYLQSIKQKWC

SEQ ID NO: 144 - protein, 31 aa, Homo sapiens
HSDAVFTDQYTRLRKQMAAKKYLQSIKQKYC

SEQ ID NO: 145 - protein, 31 aa, Homo sapiens
HSDAVFTDQYTRLRKQMAGKKYLQSIKQRIC

SEQ ID NO: 146 - protein, 31 aa, Homo sapiens
HSDAVFTDQYTRLRKQMAKKKYLQSIKQRIC

SEQ ID NO: 147 - protein, 31 aa, Homo sapiens
HSDAVFTDQYTRLRKQMASKKYLQSIKQRIC

SEQ ID NO: 148 - protein, 31 aa, Homo sapiens
HSDAVFTDQYTRLRKQMAAKKYLQSIPQRIC

SEQ ID NO: 149 - protein, 31 aa, Homo sapiens
HSDAVFTDQYTRLRKQMASKKYLQSIRQRIC

SEQ ID NO: 150 - protein, 38 aa, Homo sapiens
HSDGIFTDSYSRYRKQMAVKKYLAAVLGKRYKQRVKNK

SEQ ID NO: 151 - protein, 27 aa, Homo sapiens
HSDGIFTDSYSRYRKQMAVKKYLAAVL

SEQ ID NO: 152 - protein, 28 aa, Homo sapiens
HSDAVFTDNYTRLRKQMAVKKYLNSILN

SEQ ID NO: 153 - protein, 31 aa, Homo sapiens; identical to SEQ ID NO: 2

SEQ ID NO: 154 - protein, 31 aa, Homo sapiens
HTDAVFTDQYTRLRKQVAAKKYLQSIKQKRY

SEQ ID NO: 155 - protein, 32 aa, Homo sapiens; identical to SEQ ID NO: 113

SEQ ID NO: 156 - protein, 32 aa, Homo sapiens
HTDAVFTDQYTRLRKQVAAKKYLQSIKQKRYC

(19) United States
(12) Patent Application Publication
(10) Pub. No.: US 2009/0280106 A1
(43) Pub. Date: Nov. 12, 2009

(54) Pituitary adenylate cyclase acivating peptide (pacap) receptor (vpac2) agonists and their pharmacological methods of use

(75) Inventors: Kevin Clairmont, Cheshire, CT (US); James Whelan, Madison, CT (US); Kevin Lumb, Guilford, CT (US)

Correspondence Address: Barbara A. Shimei;Director, Patents & Licensing, Bayer HealthCare LLC - Pharmaceuticals, 555 White Plains Road, Third Floor, Tarrytown, NY, 10591, US

(73) Assignee: Bayer Pharmaceuticals Corporation, West Haven, CT (US)

(21) Appl. No.: 11/919,819

(22) PCT Filed: Apr. 18, 2006
(86) PCT No.: PCT/US2006/014808

§ 371 Date: Nov. 2, 2007

Related U.S. Application Data
(60) Provisional application No. 60/678,860, filed on May 6, 2005.

(51) Int. Cl. A61K 38/48; C07K 14/435; C07H 21/04; C12N 15/63; C12N 5/00; C12P 21/06; C07K 16/18; A61K 38/16; A61K 38/28; A61K 38/46

(52) U.S. Cl. 424/94.63; 530/324; 536/23.5; 435/320.1; 435/325; 435/69.1; 530/387.9; 514/12; 514/4; 424/94.6

(57) ABSTRACT

This invention provides peptides with novel modifications that provide suitable derivatization sites to improve the pharmacokinetic properties of the peptides. These modified peptides function in vivo as agonists of the VPAC2 receptor. The peptides of the present invention provide a new therapy for patients with decreased endogenous insulin secretion, for example, type 2 diabetics.

This application claims benefit of U.S. Provisional Application Ser. No. 60/678,860; filed on May 6, 2005, the contents of which are incorporated herein by reference in their entirety.

### FIELD OF THE INVENTION

This invention relates to novel modifications that provide suitable derivatization sites to improve the pharmacokinetic properties of the peptides. Such N-terminal modifications at the main chain amino group of the first amino acid residue or C-terminal modifications at the main chain carboxylate group of the last amino acid residue may include aliphatics, C3 to C7 cycloalkyl, aryl, or mono- or bi-cyclic heteroaromatics containing one or more nitrogen, oxygen, and/or sulfur heteroatoms. In addition, the N-terminal modifications may provide suitable derivatization sites (exemplified, but not limited to, amino and thiol groups). The modified peptides of the present invention are useful in stimulating the release of insulin from pancreatic β-cells in a glucose-dependent manner, thereby providing a treatment option for those individuals afflicted with metabolic disorders such as diabetes or impaired glucose tolerance, a prediabetic state.

### BACKGROUND OF THE INVENTION

Diabetes is characterized by impaired glucose metabolism manifesting itself, among other things, by an elevated blood glucose level in the diabetic patient. Underlying defects lead to a classification of diabetes into two major groups: type 1 diabetes, or insulin dependent diabetes mellitus (IDDM), which arises when patients lack β-cells producing insulin in their pancreatic islets of Langerhans; and type 2 diabetes, or non-insulin dependent diabetes mellitus (NIDDM), which occurs in patients with an impaired β-cell function and alterations in insulin action.

Type 1 diabetic patients are currently treated with insulin, while the majority of type 2 diabetic patients are treated with agents that stimulate β-cell function or with agents that enhance the tissue sensitivity of the patients towards insulin. Over time almost one-half of type 2 diabetic subjects lose their response to these agents and then must be placed on insulin therapy. The drugs presently used to treat type 2 diabetes are described below.

Alpha-glucosidase inhibitors (e.g., Precose®, Voglibose™, and Miglitol®) reduce the excursion of postprandial glucose by delaying the absorption of glucose from the gut. These drugs are safe and provide treatment for mild to moderately affected diabetic subjects. However, gastrointestinal side effects have been reported in the literature.

Insulin sensitizers are drugs that enhance the body's response to insulin. Thiozolidinediones such as Avandia™ (rosiglitazone) and Actos™ (pioglitazone) activate the peroxisome proliferator-activated receptor (PPAR) gamma subtype and modulate the activity of a set of genes that have not been well described. Rezulin™ (troglitazone), the first drug in this class, was withdrawn because elevated liver enzyme levels and drug induced hepatotoxicity. These hepatic effects do not appear to be a significant problem in patients using Avandia™ and Actos™. Even so, liver enzyme testing is recommended every 2 months in the first year of therapy and periodically thereafter. Avandia™ and Actos™ seem to be associated with fluid retention and edema. Another potential side effect is weight gain. Avandia™ is not indicated for use with insulin because of concern about congestive heart failure.

Insulin secretagogues (e.g., sulfonylureas (SFUs) and other agents that act by the ATP-dependent K+ channel) are another drug type presently used to treat type 2 diabetes. SFUs are standard therapy for type 2 diabetics that have mild to moderate fasting hyperglycemia. The SFUs have limitations that include a potential for inducing hypoglycemia, weight gain, and high primary and secondary failure rates. Ten to 20% of initially treated patients fail to show a significant treatment effect (primary failure). Secondary failure is demonstrated by an additional 20-30% loss of treatment effect after six months on an SFU. Insulin treatment is required in 50% of the SFU responders after 5-7 years of therapy (Scheen, et al., Diabetes Res. Clin. Pract. 6:533-543, 1989).

Glucophage™ (metformin HCl) is a biguanide that lowers blood glucose by decreasing hepatic glucose output and increasing peripheral glucose uptake and utilization. The drug is effective at lowering blood glucose in mildly and moderately affected subjects and does not have the side effects of weight gain or the potential to induce hypoglycemia. However, Glucophage™ has a number of side effects including gastrointestinal disturbances and the potential for lactic acidosis. Glucophage™ is contraindicated in diabetics over the age of 70 and in subjects with impairment in renal or liver function. Finally, Glucophage™ has primary and secondary failure rates similar to the SFUs.

Insulin treatment is instituted after diet, exercise, and oral medications have failed to adequately control blood glucose. This treatment has the drawbacks that it is an injectable, that it can produce hypoglycemia, and that it causes weight gain.

Because of the problems with current treatments, new therapies to treat type 2 diabetes are needed. In particular, new treatments to retain normal (glucose-dependent) insulin secretion are needed. Such new drugs should have the following characteristics: dependent on glucose for promoting insulin secretion (i.e., produce insulin secretion only in the presence of elevated blood glucose); low primary and secondary failure rates; and preservation of islet cell function. The strategy to develop the new therapy disclosed herein is based on the cyclic adenosine monophosphate (cAMP) signaling mechanism and its effects on insulin secretion.

Cyclic AMP is a major regulator of the insulin secretion process. Elevation of this signaling molecule promotes the closure of the K+ channels following the activation of protein kinase A pathway. Closure of the K+ channels causes cell depolarization and subsequent opening of Ca⁺⁺ channels, which in turn leads to exocytosis of insulin granules. Little if any effects on insulin secretion occurs in the absence of low glucose concentrations (Weinhaus, et al., Diabetes 47:1426-1435, 1998). Secretagogues like PACAP (pituitary adenylate cyclase activating peptide), VIP (vasoactive intestinal peptide), GIP (glucose-dependent insulinotropic polypeptide), and GLP-1 (glucagon-like peptide 1) use the cAMP system to regulate insulin secretion in a glucose-dependent fashion (Komatsu, et al., Diabetes 46:1928-1938, 1997; Filipsson, et al., Diabetes 50:1959-1969, 2001; Drucker, Endocrinology 142:521-527, 2001). Insulin secretagogues working through the elevation of cAMP such as GLP-1, VIP, GIP, and PACAP are also able to enhance insulin synthesis in addition to insulin release (Skoglund, et al., Diabetes 49:1156-1164, 2000; Borboni, et al., Endocrinology 140:5530-5537, 1999).

GLP-1 is released from the intestinal L-cell after a meal and functions as an incretin hormone (i.e., it potentiates glucose-induced insulin release from the pancreatic β-cell). It is a 37-amino acid peptide that is differentially expressed by the glucagon gene, depending upon tissue type. The clinical data that support the beneficial effect of raising cAMP levels in β-cells have been collected with GLP-1. Infusions of GLP-1 in poorly controlled type 2 diabetics normalized their fasting blood glucose levels (Gutniak, et al., New Eng. J. Med. 326:1316-1322, 1992) and with longer infusions improved the β-cell function to those of normal subjects (Rachman, et al., Diabetes 45:1524-1530, 1996). A recent report has shown that GLP-1 improves the ability of β-cells to respond to glucose in subjects with impaired glucose tolerance (Byrne, et al., Diabetes 47:1259-1265, 1998). All of these effects, however, are short-lived because of the short half-life of the peptide.

Amylin Pharmaceuticals is conducting Phase III trials with Exendin-4 (AC2993), a 39 amino acid peptide originally identified in Gila Monster. Amylin has reported that clinical studies demonstrated improved glycemic control in type 2 diabetic patients treated with Exendin-4. However, the incidence of nausea and vomiting was significant.

Glucose-dependent insulinotropic polypeptide (GIP) is a 42-residue gut peptide involved in the regulation of fat and glucose metabolism, with the insulinotropic function localized to residues 1-30. GIP is degraded by DPPIV proteolysis and eliminated by renal clearance. Limited clinical data have been collected with GIP. IV administration or continuous in type 2 diabetics caused an aute increase in plasma insulin levels (Kindmark et al., J. Clin. Endocrinol. Metab. 86: 2015-2019, 2001; Meier et al., Diabetes 53 (Suppl 3): S220-S224, 2004). These effects, however, are short-lived because of the short half-life of the peptide.

PACAP is a potent stimulator of glucose-dependent insulin secretion from pancreatic β-cells. Three different PACAP receptor types (PAC1, VPAC1, and VPAC2) have been described (Harmar, et al., Pharmacol. Reviews 50:265-270, 1998; Vaudry, et al., Pharmacol. Reviews 52:269-324, 2000). PACAP displays no receptor selectivity, having comparable activities and potencies at all three receptors. PAC1 is located predominately in the CNS, whereas VPAC1 and VPAC2 are more widely distributed. VPAC1 is located in the CNS as well as in liver, lungs, and intestine. VPAC2 is located in the CNS, pancreas, skeletal muscle, heart, kidney, adipose tissue, testis, and stomach. Recent work argues that VPAC2 is responsible for the insulin secretion from β-cells (Inagaki, et al., Proc. Natl. Acad. Sci. USA 91:2679-2683, 1994; Tsutsumi, et al., Diabetes 51:1453-1460, 2002). This insulinotropic action of PACAP is mediated by the GTP binding protein Gs. Accumulation of intracellular cAMP in turn activates the nonselective cation channels in β-cells increasing [Ca++], and promotes exocytosis of insulin-containing secretory granules.

PACAP is the newest member of the superfamily of metabolic, neuroendocrine, and neurotransmitter peptide hormones that exert their action through the cAMP-mediated signal transduction pathway (Arimura, Regul. Peptides 37:287-303, 1992). The biologically active peptides are released from the biosynthetic precursor in two molecular forms, either as a 38-amino acid peptide (PACAP-38) and/or as a 27-amino acid peptide (PACAP-27) with an amidated carboxyl termini (Arimura, supra).

The highest concentrations of the two forms of the peptide are found in the brain and testis (Arimura, supra). The shorter form of the peptide, PACAP-27, shows 68% structural homology to vasoactive intestinal polypeptide (VIP). However, the distribution of PACAP and VIP in the central nervous system suggests that these structurally related peptides have distinct neurotransmitter functions (Koves, et al., Neuroendocrinology 54:159-169, 1991).

Recent studies have demonstrated diverse biological effects of PACAP-38, from an ability to stimulate insulin secretion (Yada, et al., J. Biol. Chem. 269:1290-1293, 1994) to a role in reproduction (McArdle, Endocrinology 135:815-817, 1994). In addition, PACAP appears to play a role in hormonal regulation of lipid and carbohydrate metabolism (Gray, et al., Mol. Endrocrinol. 15:1739-47, 2001); circadian function (Harmar, et al., Cell 109: 497-508, 2002); the immune system, including autoimmune diseases (e.g., systemic lupus erythematosus); growth; energy homeostasis; regulation of appetite (Tachibana, et al., Neurosci. Lett. 339:203-206, 2003); male reproductive function (Asnicar, et al., Endrocrinol. 143:3994-4006, 2002), including erectile dysfunction (Hafez, et al., Arch. Androl. 51:15-31, 2005); neuroprotection (Zusev, et al., Regul. Pept. 123:33-41, 2004); acute and chronic inflammatory diseases, chronic obstructive pulmonary disease (COPD), septic shock, (Pozo, Trends Mol. Med. 9:211-217, 2003), and HIV infection.

Vasoactive intestinal peptide (VIP) is a 28 amino acid peptide that was first isolated from hog upper small intestine (Said and Mutt, Science 169:1217-1218, 1970; U.S. Pat. No. 3,879,371). This peptide belongs to a family of structurally-related, small polypeptides that includes helodermin, secretin, the somatostatins, and glucagon. The biological effects of VIP are mediated by the activation of membrane-bound receptor proteins that are coupled to the intracellular cAMP signaling system. These receptors were originally known as VIP-R1 and VIP-R2, however, they were later found to be the same receptors as VPAC1 and VPAC2. VIP displays comparable activities and potencies at VPAC1 and VPAC2.

To improve the stability of VIP in human lung fluid, Bolin, et al., (Biopolymers 37:57-66, 1995) made a series of VIP variants designed to enhance the helical propensity of this peptide and reduce proteolytic degradation. Substitutions were focused on positions 8, 12, 17, and 25-28, which were implicated to be unimportant for receptor binding. Moreover, the “GGT” sequence was tagged onto the C-terminus of VIP muteins with the hope of more effectively capping the helix. Finally, to further stabilize the helix, several cyclic variants were synthesized (U.S. Pat. No. 5,677,419). Although these efforts were not directed toward receptor selectivity, they yielded two analogs that have greater than 100-fold VPAC2 selectivity (Gourlet, et al., Peptides 18:403-408, 1997; Xia, et al., J. Pharmacol. Exp. Ther., 281:629-633, 1997).

There exists a need for improved peptides that have the glucose-dependent insulin secretagogue activity of PACAP, VIP, GIP, GLP-1, or Exendin-4, but with fewer side-effects, and preferably which are stable in formulation and have long plasma half-lives in vivo. Such improved in vivo half-life results from peptides with both decreased clearance and decreased susceptibility to proteolysis. Furthermore, tighter control of plasma glucose levels may prevent long-term diabetic complications. Thus, new diabetic drugs should provide an improved quality of life for patients.

### SUMMARY OF THE INVENTION

The present invention provides novel modifications that provide suitable derivatization sites to Improve the pharmacokinetic properties of the peptides. Such N-terminal modifications at the amino group of the first peptide residue may include aliphatics, C₃ to C₇ cycloalkyl, aryl, or mono- or bi-cyclic heteroaromatics containing one or more nitrogen, oxygen, and/or sulfur heteroatoms. In addition, the N-terminal modifications may provide suitable derivatization sites (exemplified, but not limited to, amino and thiol groups). Several examples of such N-terminal modifications include, but are not limited to, 2-amino benzoic acid, 3-amino benzoic acid, 4-amino benzoic acid, 4-amino-2-chloro-benzoic acid, 4-amino-3-methoxy-benzoic acid, 4-amino-3-methyl-benzoic acid, 1-amino-cyclopentane-3-carboxylic acid, trans-3-aminocyclohexane carboxylic acid, D-pipecolinic acid, 4-amino-1-methyl-1H-imidazole-2-carboxylic acid, 4-methylhiobenzoic acid, 2-methylthiobenzoic acid, 2-methylthionicotinic acid, proline, 6-aminohexanoic acid, benzoic acid, (S)-tetrahydroisoquinoline acetic acid, indoline-2-carboxylic acid, cis-3-aminocyclohexane carboxylic acid, L-pipecolinic acid, 9-gluorenylmethoxycarbonyl, 2-thio-polyethylene glycol benzoic acid, 2-thio-polyethylene glycol nicotinic acid, 4-amino-1-methyl-1H-imidazole-2-carboxylic acid, 1-amino-cyclopentane-3-carboxylic acid, 4-amino-1-methyl-1H-imidazole-2-carboxylic acid, 1-amino-cyclopentane-3-carboxylic acid, 1-amino-cyclopentane-3-carboxylic acid, (2-mercapto-1H-benzimidazol-1-yl)acetic acid, 2-(tritylthio)ethyl]amino}nicotinate, 2-{[2-(tritylthio)ethyl]amino}nicotinate, 1-[2-(tritylthio)ethyl]-1H-imidazole-2-carboxylate, 4-{[2-(tritylthio)ethyl]amino}pyrimidine-5-carboxylate, 1-[2-(tritylthio)ethyl]-1H-benzimidazole-2-carboxylate, 2-mercapto-1H-imidazol-1-yl)acetic acid, ({1-[2-(tritylthio)ethyl]-1H-imidazol-2-yl}thio)acetate, 3-(2-tritylsulfanylethylamino)pyrazine-2-carboxylate, 4-mercaptothiazole-5-carboxylic acid, 2-mercaptothiazole-5-carboxylic acid, 2-(2-tritylsulfanylethylamino)thiazole-5-carboxylae, 2-mercapto-6-methylpyrimidine-4-carboxylic acid, 5-mercaptonicotinic acid, 5-isopropyl-2-mercaptothiazole-4-carboxylic acid, 1-hexadecyl-1H-benzoimidazol-2-ylsulfanyl)acetic acid, and 2-(2-tert-butoxycarbonylaminoethylamino)thiazole-5-carboxylic acid.

The invention relates to a peptide of Formula (I)

Z1-A1-A2-A3-A4-A5-Phe-Thr-A8-A9-A10-A11-A12-A13-Arg-A15-A16-A17-Ala-A19-A20-

A21-Tyr-Leu-A24-A25-A26-A27-A28-A29-A30-A31-A32-A33-A34-A35-A36-A37-A38-A39-

A40-Z2 (SEQ ID NO: 1)

wherein
A1 is His or Ala;
A2 is Ser, Thr, or Ala;
A3 is Asp or Glu;
A4 is Ala or Gly;
A5 is Val or Ile;
A8 is Asp, Glu, or Ala;
A9 is Gln, Asn, Ser, or Ala;
A11 is Thr or Ser;
A12 is Arg or Lys;
A13 is Leu or Tyr;
A15 is Lys or Ala;
A16 is Gln or Ala;
A17 is Val, Met, Leu, Nle, or Ala;
A19 is Ala, Val, Gly, Lys, Arg, Ser, Glu, Phe, Ile, Leu, Met, Thr, or Trp;
A20 is Lys or His;
A21 is Lys, His, or Ala;
A24 is Gln, Asn, or Ala;
A25 is Ser, Asp, Thr, or Ala;
A26 is Ile, Val, Leu, or Ala;
A27 is any amino acid;
A28 is Gln, Asn, Gly, Ala, or Lys;
A29 is Lys, Gly, Arg, Cys, Ala, Asp, Glu, His, Ile, Leu, Met, Asn, Pro, Gln, Arg, Ser, Thr or deleted;
A30 is any amino acid or deleted;
A31 is Tyr, Thr, Cys or deleted;
A32 is Lys, Cys, Lys-X, Cys-PEG or deleted;
A33 is Gln, Lys, Cys, Lys-X, Cys-PEG or deleted;
A34 is Arg, Lys, Cys, Lys-X, Cys-PEG or deleted;
A35 is Val, Lys, Cys, Lys-X, Cys-PEG or deleted;
A36 is Lys, Cys, Lys-X, Cys-PEG or deleted;
A37 is Asn, Lys, Cys, Lys-X, Cys-PEG or deleted;
A38 is Lys, Cys, Lys-X, Cys-PEG or deleted; and
A39 is Lys, Cys, Lys-X, Cys-PEG or deleted.

Lys-X is Lys modified at N^{B} with a fatty acid exemplified by CH₃(CH₂)ₙCOOH where n ranges from 0 to about 24.

Z1 is selected from

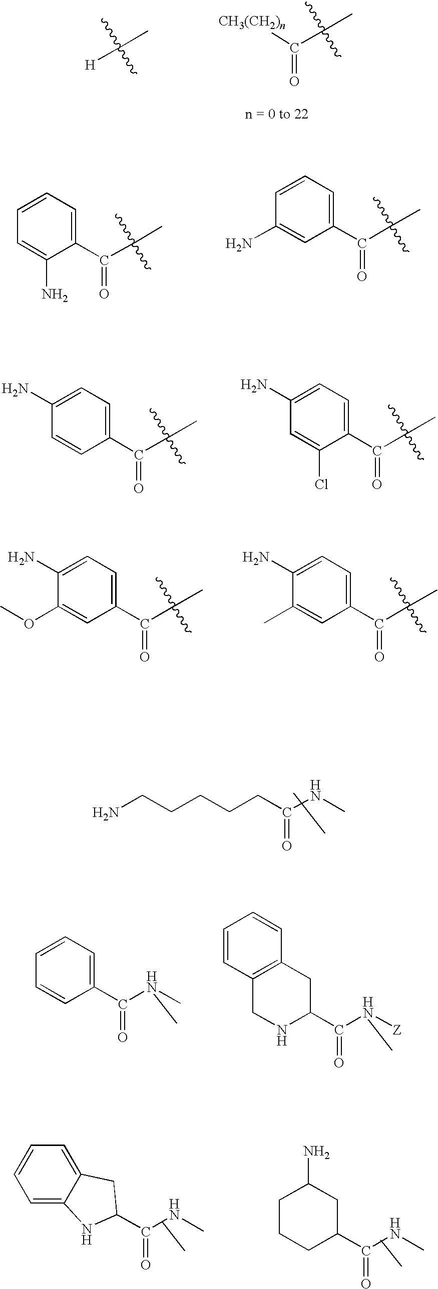

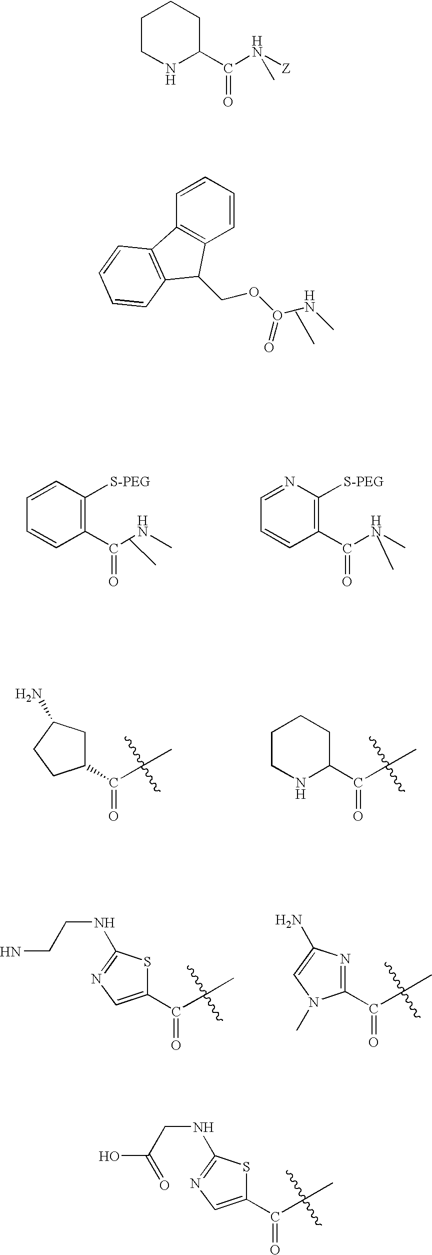

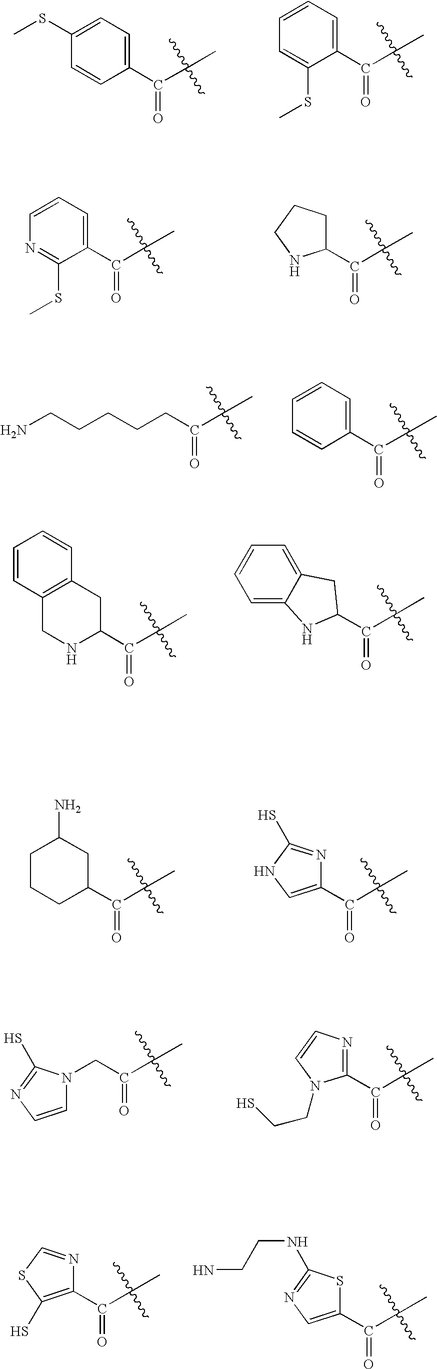

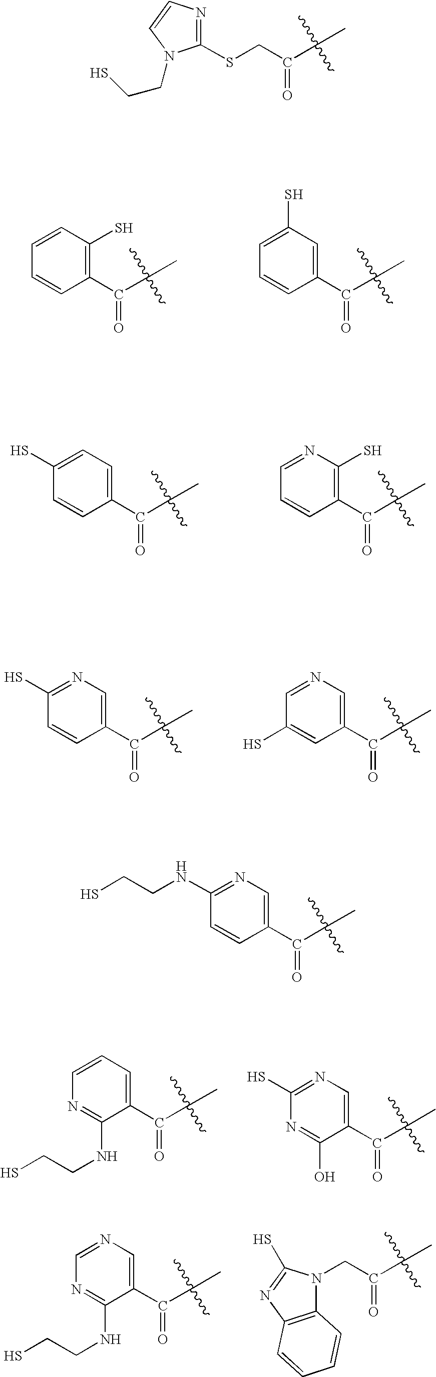

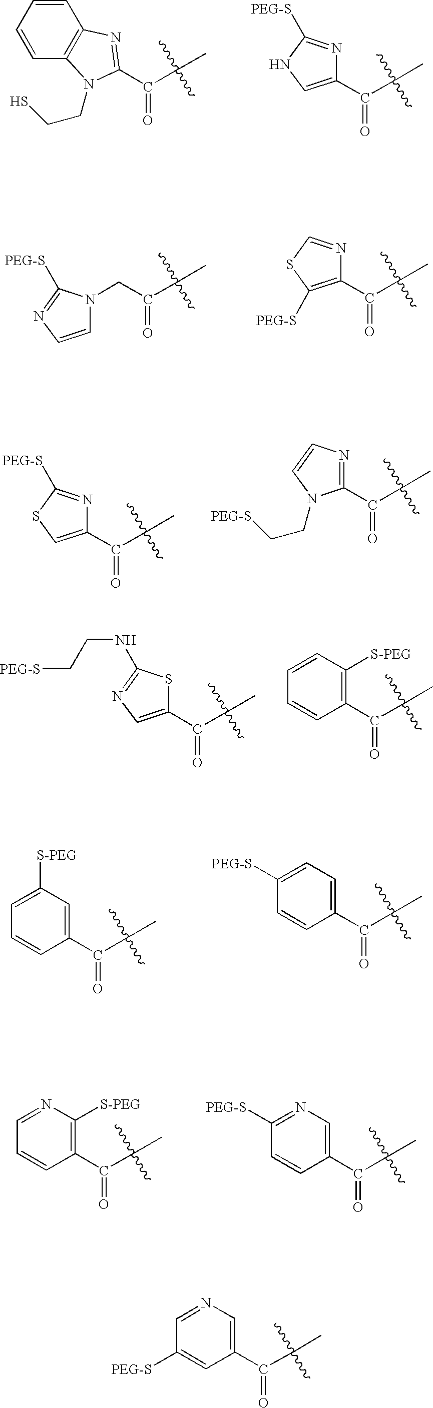

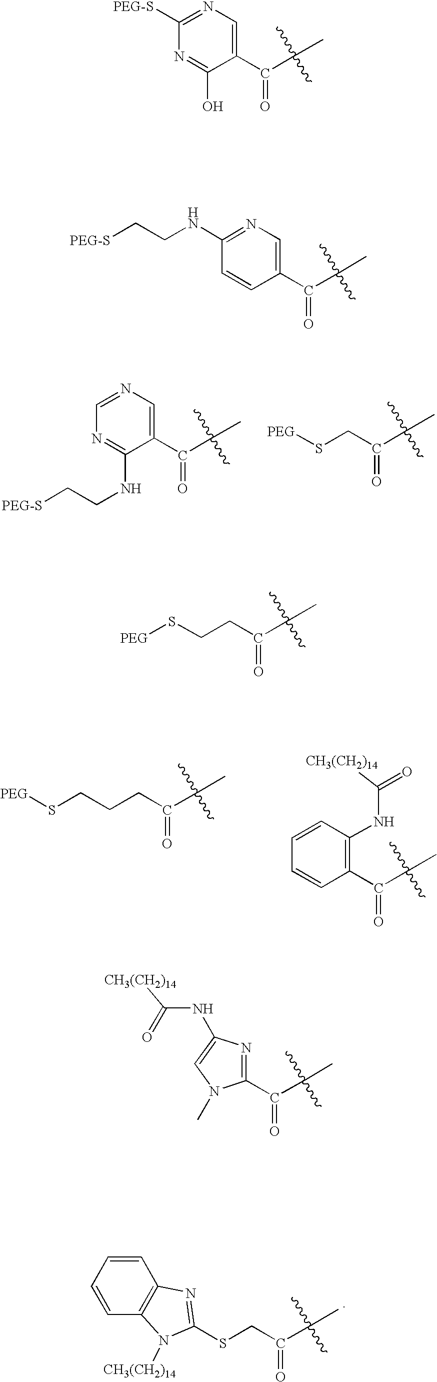

Z2 may be a hydroxyl group such that the peptide has an unmodified carboxylate C-terminus or Z2 may be a modification of the C-terminal carboxylate group. Z2 may be a modification such as amidation, or Z2 may also be an unnatural amino acid or amide. Z2 may be exemplified by, but not limited to,

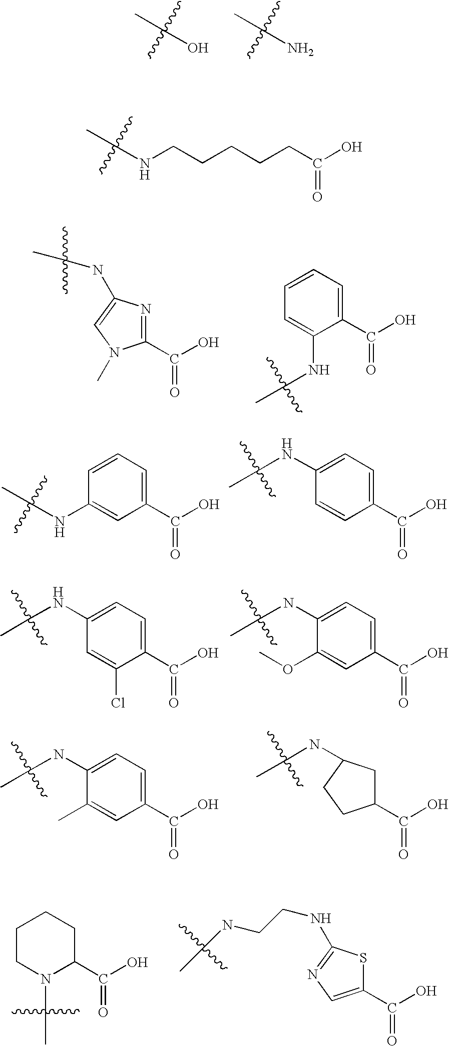

For the peptide of Formula (I), the N-terminal modifications may be attached via an amide bond to the alpha-amino group of the first amino acid of said peptide. The C-terminal modifications may be attached via an amide bond to the main chain carboxylate group of the last amino acid of said peptide. Examples of peptides of Formula (I) may be found in, but are not limited to, the peptides described in Table 1 (e.g., SEQ ID NO: 1-156).

Derivatives of the present invention may include peptides that have been fused with another compound, such as a compound to increase the half-life of the peptide and/or to reduce potential immunogenicity of the peptide (e.g., polyethylene glycol, “PEG”). For example, PEGylated peptides typically have greater half-life in vivo (Greenwald, Adv. Drug. Del. Rev. 55:217-250, 2003).

In the case of PEGylation, the fusion of the peptide to PEG may be accomplished by any means known to one skilled in the art. For example, PEGylation may be accomplished by first introducing a cysteine mutation into the peptide to provide a linker upon which to attach the PEG, followed by site-specific derivatization with PEG-maleimide. Alternatively, the N-terminal modification may incorporate a reactive moiety for coupling to PEG, as exemplified by the amine group, the mercapto group, or the carboxylate group of the N-terminal modifying compounds disclosed above. For example, PEGylation may be accomplished by first introducing a mercapto moiety into the peptide via the N-terminal modifying group to provide a linker upon which to attach the PEG, followed by site-specific derivatization with methoxy-PEG-maleimide reagents supplied by, for example, either Nektar Therapeutics (San Carlos, Calif., USA) and/or NOF (Tokyo, Japan). In addition to maleimide, numerous Cys reactive groups are known to those skilled in the art of protein cross-linking, such as the use of alkyl halides and vinyl sulfones (see, e.g., Proteins, Structure and Molecular Properties, 2nd ed., T. E. Creighton, W.H. Freeman and Company, New York, 1993). In addition, the PEG could be introduced by direct attachment to the C-terminal carboxylate group, or to an internal amino acid such as Cys, Lys, Asp, or Glu or to unnatural amino acids that contain similar reactive sidechain moieties.

Various size PEG groups can be used, as exemplified but not limited to, PEG polymers of from about 5 kDa to about 43 kDa. The PEG modification may include a single, linear PEG. For example, linear 5, 20, or 30 kDa PEGs that are attached to maleidmide or other cross-linking groups are available from Nektar and/or NOF (see, e.g., Table 2). Also, the modification may involve branched PEGs that contain two or more PEG polymer chains that are attached to maleimide or other cross-linking groups are available from Nektar and NOF (see, e.g., Table 2).

It is possible that PEGylation with a smaller PEG (e.g., a linear 5 kDa PEG) will less likely reduce activity of the peptide, whereas a larger PEG (e.g., a branched 40 kDa PEG) will more likely reduce activity. However, a larger PEG will increase plasma half-life further so that once a week injection may be possible (Harris, et al., Clin. Pharmacokinet. 40:539-551, 2001).

The linker between the PEG and the peptide cross-linking group can be varied. For example, the commercially available thiol-reactive 40 kDa PEG (mPEG2-MAL) from Nektar (Huntsville, Ala.) employs a maleimide group for conjugation to Cys, and the maleimide group is attached to the PEG via a linker that contains a Lys (see, e.g., Table 2). As a second example, the commercially available thiol-reactive 43 kDa PEG (GL2-400MA) from NOF employs a maleimide group for conjugation to Cys, and the maleimide group is attached to the PEG via a bi-substituted alkane linker (see, e.g., Table 2). In addition, the PEG polymer can be attached directly to the maleimide, as exemplified by PEG reagents of molecular weight 5 and 20 kDa available form Nektar Therapeutics (Huntsville, Ala.) (see, e.g., Table 2).

The present invention exemplifies, but is not limited to, the use of a mercapto group as a cross-linking site. It is well known that other moieties present in amino acids such as the amino group of the N-terminal modifying compound, the C-terminal carboxylate of either an unmodified C-terminus or a peptide modified with Z2, and the side chains of amino acids such as Lys, Arg, Asp, and Glu provide reactive groups that provide moieties suitable for covalent modification and attachment to PEG. Numerous examples of suitable cross-linking agents are known to those skilled in the art (see, e.g., Proteins, Structure and Molecular Properties, 2nd ed., T. E. Creighton, W.H. Freeman and Company, New York, 1993). Such cross-linking agents can be linked to PEG as exemplified by, but not limited to, commercially available PEG derivatives containing amines, aldehydes, acetals, maleimide, succinimides, and thiols that are marketed, for example, by Nektar and NOF (e.g., Harris, et al., Clin. Pharmokinet. 40:539-551, 2001).

In addition to PEGylation, the peptides of the present invention may be modified with fatty acids that improve pharmacodynamic properties. For example, the amine containing N-terminal modifying compounds can be derivatized with palmitate or myristolate or other fatty acids using methods known to those skilled in the art or an alkyl (e.g., C₆-C₁₈) moiety can be included directly as part of the N-terminal modifying compound.

The peptides of the present invention have improved stability to proteolysis by DPPIV and in plasma as compared to PACAP or VIP. While both VIP and PACAP27 have been reported to be resistant to cleavage by DPPIV (Zhu, et al., J. Biol. Chem. 278: 22418-22423, 2003), the inventors have demonstrated that these peptides are cleaved at longer time points. The derivatives of the present invention demonstrate an extended duration of action in vivo, supporting a dosing interval of less than once per day or once per week or greater, when derivatized.

The peptides of the present invention (e.g., Table 1) provide a new therapy for patients with, for example, metabolic disorders such as those resulting from decreased endogenous insulin secretion, in particular type 2 diabetics, or for patients with impaired glucose tolerance, a prediabetic state that has a mild alteration in insulin secretion. In addition, the peptides of the present invention may be useful in the prevention and/or treatment of type 1 diabetes, gestational diabetes, maturity-onset diabetes of the young (MODY), latent autoimmune diabetes adult (LADA), and associated diabetic dyslipidemia and other diabetic complications, as well as hyperglycemia, hyperinsulinemia, impaired glucose tolerance, impaired fasting glucose, dyslipidemia, hypertriglyceridemia, Syndrome X, and insulin resistance.

The peptides of the present invention (e.g., Table 1) may also be utilized in the prevention and/or treatment of obesity (e.g., regulation of appetite and food intake); disorders of energy homeostasis; disorders of lipid and carbohydrate metabolism; cardiovascular disease, including atherosclerosis, coronary heart disease, coronary artery disease, hyperlipidemia, hypercholesteremia, low HDL levels and hypertension; cerebrovascular disease and peripheral vessel disease; polycystic ovary syndrome; carcinogenesis, and hyperplasia; asthma and chronic obstructive pulmonary disease; male reproduction problems (including erectile dysfunction); ulcers; neurodegenerative diseases (including Parkinson's and Alzheimer's); sleep disorders and circadian dysfunction; growth disorders; immune diseases, including autoimmune diseases (e.g., systemic lupus erythematosus); chronic inflammatory diseases; septic shock; HIV infection and AIDS, and other conditions identified herein, or function otherwise as described later herein.

One aspect of the invention is a peptide of Formula (I), and fragments, derivatives, and variants thereof that demonstrate at least one biological function that is substantially the same as the peptides of Formula (I) (collectively, “peptides of this invention”), including functional equivalents thereof (e.g., Table 1).

Antibodies and antibody fragments that selectively bind the peptides of this invention (e.g., Table 1) are also provided. Such antibodies are useful in detecting the peptides of this invention, and can be identified and made by procedures well known in the art. A polyclonal N-terminal IgG antibody and a monoclonal C-terminal Fab antibody have been generated which recognize peptides of this invention.

The invention is also directed to a method of treating diabetes, diabetes-related disorders, and/or other diseases or conditions affected by the peptides of this invention, for example, effected by the VPAC2 agonist function of the peptides of this invention, in a mammal, comprising administering a therapeutically effective amount of any of the peptides of the present invention or any peptide active at VPAC2 such as a peptide of Formula (I) to said mammal (e.g., Table 1).

Also disclosed are methods of making the peptides of this invention.

### DETAILED DESCRIPTION OF THE INVENTION

This invention provides novel modified peptides, and fragments, derivatives, and variants thereof that demonstrate at least one biological function that is substantially the same as the peptides of Formula (I) (peptides of this invention). The peptides of this invention (e.g., Table 1) function in vivo as VPAC2 agonists or otherwise in the prevention and/or treatment of such diseases or conditions as diabetes including both type 1 and type 2 diabetes, gestational diabetes, maturity-onset diabetes of the young (MODY) (Herman, et al., Diabetes 43:40, 1994); latent autoimmune diabetes adult (LADA) (Zimmet, et al., Diabetes Med. 11:299, 1994); and associated diabetic dyslipidemia and other diabetic complications, as well as hyperglycemia, hyperinsulinemia, impaired glucose tolerance, impaired fasting glucose, dyslipidemia, hypertriglyceridemia, Syndrome X, and insulin resistance.

The peptides of the present invention (e.g., Table 1) may also be utilized in the prevention and/or treatment of obesity (e.g., regulation of appetite and food intake); disorders of energy homeostasis; disorders of lipid and carbohydrate metabolism; cardiovascular disease, including atherosclerosis, coronary heart disease, coronary artery disease, hyperlipidemia, hypercholesteremia, low HDL levels and hypertension; cerebrovascular disease and peripheral vessel disease; polycystic ovary syndrome; carcinogenesis, and hyperplasia; asthma and chronic obstructive pulmonary disease; male reproduction problems (including erectile dysfunction); ulcers; neurodegenerative diseases (including Parkinson's and Alzheimer's); sleep disorders and circadian dysfunction; growth disorders; immune diseases, including autoimmune diseases (e.g., systemic lupus erythematosus); chronic inflammatory diseases; septic shock; HIV infection and AIDS, and other conditions identified herein, or function otherwise as described later herein.

The peptides of this invention (e.g., Table 1) will stimulate insulin release from pancreatic β-cells in a glucose-dependent fashion. Furthermore, the peptides of this invention are stable in both aqueous and non-aqueous formulations and exhibit a plasma half-life of greater than one hour, for example, demonstrating a plasma half-life greater than 6 hours.

The peptides of this invention are VPAC2 agonists (e.g., Table 1). Furthermore, the peptides of this invention stimulate insulin release into plasma in a glucose-dependent fashion without inducing a stasis or increase in the level of plasma glucose that is counterproductve to the treatment of, for example, type 2 diabetes. Additionally, the peptides of this invention may be selective agonists of the VPAC2 receptor, thereby causing, for example, an increase in insulin release into plasma, while being selective against other receptors that are responsible for such disagreeable or dangerous side effects as gastrointestinal water retention, and/or unwanted cardiovascular effects such as increased heart rate or blood pressure.

The peptides of this invention are also stable in aqueous and non-aqueous formulations. The peptides of this invention will exhibit less than 10% degradation at 37-40° C. over a period of one week, when dissolved in water (at pH between 7-8) or non-aqueous organic solvent, or the peptides of this invention will exhibit less than 5% degradation at 37-40° C. over a period of one week, when dissolved in water (at pH between 7-8) or non-aqueous organic solvent. Furthermore, compositions and formulations of the present invention may comprise peptides of the present invention and about 2% to about 30% DMSO. In another embodiment of the present invention, the compositions and formulations may optionally include about 0.2% to about 3% (w/v) of additional solvents such as propylene glycol, dimethyl formamide, propylene carbonate, polyethylene glycol, and triglycerides.

Finally, the derivatized peptides of this Invention may exhibit a plasma half-life of, for example, 1 hour in rats after IV injection, 3 hours, or 6 hours. Furthermore, the derivatized peptide may demonstrate a significant lowering of the plasma glucose AUC following subcutaneous injection in rats, for example, 24 hours, 41 hours, or 65 hours following injection.

The peptides of this invention provide a new therapy for patients with decreased endogenous insulin secretion or impaired glucose tolerance, in particular, type 2 diabetes. That is, the peptides of the present invention are long-acting VPAC2 receptor agonists that may be used to maintain, improve, and restore glucose-stimulated insulin secretion. Furthermore, a selective peptide agonist of the VPAC2 receptor will enhance glucose-dependent insulin secretion in the pancreas without causing the side effects associated with non-selective activation of the other PACAP receptors.

Certain terms used throughout this specification are defined below, and others will be defined as introduced. The single letter abbreviation for a particular amino acid, its corresponding amino acid, and three letter abbreviation are as follows: A, alanine (Ala); C, cysteine (Cys); D, aspartic acid (Asp); E, glutamic acid (Glu); F, phenylalanine (Phe); G, glycine (Gly); H, histidine (His); I, isoleucine (Ile); K, lysine (Lys), L, leucine (Leu); M, methionine (Met); N, asparagine (Asn); P, proline (Pro); Q, glutamine (Gln); R, arginine (Arg); S, serine (Ser); T, threonine (Thr); V, valine (Val); W, tryptophan (Trp); Y, tyrosine (Tyr); and norleucine (Nle).

“Functional equivalent” and “substantially the same biological function or activity” each means that degree of biological activity that is within about 30% to about 100% or more of that biological activity demonstrated by the peptide to which it is being compared when the biological activity of each peptide is determined by the same procedure.

“Biological activity,” “activity,” or “biological function,” which are used interchangeably, herein mean an effector function that is directly or indirectly performed by a peptide (whether in its native or denatured conformation), or by any fragments, derivatives, and variants thereof. Biological activities include, for example, binding to peptides, binding to other proteins or molecules, activity as a DNA binding protein, as a transcription regulator, ability to bind damaged DNA, etc.

The terms “fragment,” “derivative,” and “variant,” when referring to the peptides of the present invention, means fragments, derivatives, and variants of the peptides which retain substantially the same biological function or activity as such peptides, as described further below.

A fragment is a portion of the peptide which retains substantially similar functional activity, for example, as described in the in vivo models disclosed herein.

A derivative includes all modifications to the peptide which substantially preserve the functions disclosed herein and include additional structure and attendant function (e.g., modified N-terminus peptides, modified C-terminus peptides, or PEGylated peptides), fusion peptides which confer targeting specificity or an additional activity such as toxicity to an intended target, as described further below.

The peptides of the present invention may be synthetic peptides.

The fragment, derivative, or variant of the peptides of the present invention may be (i) one in which one or more of the amino acid residues are substituted with a conserved or non-conserved amino acid residue and such substituted amino acid residue may or may not be one encoded by the genetic code, or (ii) one in which one or more of the amino acid residues includes a substituent group, or (iii) one in which the mature peptide is fused with another compound, such as a compound to increase the half-life of the peptide (e.g., polyethylene glycol), or (iv) one in which the additional amino acids are fused to the mature peptide, such as a leader or secretory sequence or a sequence which is employed for purification of the mature peptide, or (v) one in which the peptide sequence is fused with a larger peptide (e.g., human albumin, an antibody or Fc, for increased duration of effect). Such fragments, derivatives, and variants and analogs are deemed to be within the scope of those skilled in the art from the teachings herein.

The derivatives of the present invention may contain conservative amino acid substitutions (defined further below) made at one or more nonessential amino acid residues. A “nonessential” amino acid residue is a residue that can be altered from the wild-type sequence of a protein without altering the biological activity, whereas an “essential” amino acid residue is required for biological activity. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Fragments, or biologically active portions include peptide fragments suitable for use as a medicament, to generate antibodies, as a research reagent, and the like. Fragments include peptides comprising amino acid sequences sufficiently similar to or derived from the amino acid sequences of a peptide of this invention and exhibiting at least one activity of that peptide, but which include fewer amino acids than the full-length peptides disclosed herein. Typically, biologically active portions comprise a domain or motif with at least one activity of the peptide. A biologically active portion of a peptide can be a peptide which is, for example, five or more amino acids in length. Such biologically active portions can be prepared synthetically or by recombinant techniques and can be evaluated for one or more of the functional activities of a peptide of this invention by means disclosed herein and/or well known in the art.

Variants of the peptides of this invention include peptides having an amino acid sequence sufficiently similar to the amino acid sequence of the peptides of this invention or a domain thereof. The term “sufficiently similar” means a first amino acid sequence that contains a sufficient or minimum number of identical or equivalent amino acid residues relative to a second amino acid sequence such that the first and second amino acid sequences have a common structural domain and/or common functional activity. For example, amino acid sequences that contain a common structural domain that is at least about 45%, about 75% through 98%, identical are defined herein as sufficiently similar. Variants will be sufficiently similar to the amino acid sequence of the peptides of this invention. Such variants generally retain the functional activity of the peptides of this invention.

Variants include peptides that differ in amino acid sequence due to mutagenesis. Variants that function as VPAC receptor agonists may be identified by screening combinatorial libraries of mutants, for example truncation mutants, of the peptides of this invention for VPAC receptor agonist activity.

The invention also provides chimeric or fusion peptides. The targeting sequence is designed to localize the delivery of the peptide to minimize potential side effects. The peptides of this invention may be composed of amino acids joined to each other by peptide bonds or modified peptide bonds (i.e., peptide isosteres), and may contain amino acids other than the 20 gene-encoded amino acids. The peptides may be modified by either natural processes, such as posttranslational processing, or by chemical modification techniques which are well known in the art. Such modifications are well described in basic texts and in more detailed monographs, as well as in a voluminous research literature. Modifications may occur anywhere in a peptide, including the peptide backbone, the amino acid side-chains, and the amino or carboxyl termini. It will be appreciated that the same type of modification may be present in the same or varying degrees at several sites in a given peptide. Also, a given peptide may contain many types of modifications. Peptides may be branched, for example, as a result of ubiquitination, and they may be cyclic, with or without branching. Cyclic, branched, and branched cyclic peptides may result from posttranslation natural processes or may be made by synthetic methods. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formulation, gamma-carboxylation, glycosylation, GPI anchor formation, hydroxylation, iodination, methylation, myristoylation, oxidation, PEGylation, proteolytic processing, phosphorylation, prenylation, racemization, selenoylation, sulfation, transfer-RNA mediated addition of amino acids to proteins such as arginylation, and ubiquitination (see, e.g., Proteins, Structure and Molecular Properties 2nd ed., T. E. Creighton, W.H. Freeman and Company, New York (1993); Posttranslational Covalent Modification of Proteins, B. C. Johnson, ed., Academic Press, New York, pgs. 1-12 (1983); Seifter, et al., Meth. Enzymol. 182:626-646, 1990; Rattan, et al., Ann. N.Y. Acad. Sci. 663:48-62, 1992).

The peptides of the present invention include the peptides of Formula (I) (e.g., Table 1), as well as those sequences having insubstantial variations in sequence from them. An “insubstantial variation” would include any sequence addition, substitution, or deletion variant that maintains substantially at least one biological function of the peptides of this invention, for example, VPAC receptor agonist activity, and/or enhancement of insulin secretion or lowering of blood glucose demonstrated herein. These functional equivalents may include peptides which have at least about 90% identity to the peptides of the present invention, at least 95% identity to the peptides of the present invention, and at least 99% identity to the peptides of the present invention, and also include portions of such peptides having substantially the same biological activity. However, any peptide having insubstantial variation in amino acid sequence from the peptides of the present invention that demonstrates functional equivalency as described further herein is included in the description of the present invention.

The peptides of this invention may be a product of chemical synthetic procedures.

The peptides of this invention may be conveniently isolated by methods that are well known in the art. Purity of the preparations may also be assessed by any means known in the art, such as SDS-polyacrylamide gel electrophoresis and mass spectroscopy and liquid chromatography.

Also provided are related peptides within the understanding of those with skill in the art, such as chemical mimetics, organomimetics, or peptidomimetics. As used herein, the terms “mimetic,” “peptide mimetic,” “peptidomimetic,” “organomimetic,” and “chemical mimetic” are intended to encompass peptide derivatives, peptide analogs, and chemical compounds having an arrangement of atoms in a three-dimensional orientation that is equivalent to that of a peptide of the present invention. It will be understood that the phrase “equivalent to” as used herein is intended to encompass peptides having substitution(s) of certain atoms, or chemical moieties in said peptide, having bond lengths, bond angles, and arrangements in the mimetic peptide that produce the same or sufficiently similar arrangement or orientation of said atoms and moieties to have the biological function of the peptides of the invention. In peptide mimetics, the three-dimensional arrangement of the chemical constituents may be structurally and/or functionally equivalent to the three-dimensional arrangement of the peptide backbone and component amino acid sidechains in the peptide, resulting in such peptido-, organo-, and chemical mimetics of the peptides of the invention having substantial biological activity. These terms are used according to the understanding in the art, as illustrated, for example, by Fauchere, (Adv. Drug Res. 15:29, 1986); Veber & Freidinger, (TINS p. 392, 1985); and Evans, et al., (J. Med. Chem. 30:1229, 1987), incorporated herein by reference.

It is understood that a pharmacophore exists for the biological activity of each peptide of the invention. A pharmacophore is understood in the art as comprising an idealized, three-dimensional definition of the structural requirements for biological activity. Peptido-, organo-, and chemical mimetics may be designed to fit each pharmacophore with current computer modeling software (computer aided drug design). Said mimetics may be produced by structure-function analysis, based on the positional information from the substituent atoms in the peptides of the invention.

Peptides as provided by the invention may be advantageously synthesized by any of the chemical synthesis techniques known in the art, particularly solid-phase synthesis techniques, for example, using commercially-available automated peptide synthesizers. The mimetics of the present invention may be synthesized by solid phase or solution phase methods conventionally used for the synthesis of peptides (see, e.g., Merrifield, J. Amer. Chem. Soc. 85:2149-54, 1963; Carpino, Acc. Chem. Res. 6:191-98, 1973; Birr, Aspects of the Merrifield Peptide Synthesis, Springer-Verlag: Heidelberg, 1978; The Peptides: Analysis, Synthesis, Biology, Vols. 1, 2, 3, and 5, (Gross & Meinhofer, eds.), Academic Press: New York, 1979; Stewart, et al., Solid Phase Peptide Synthesis, 2nd. ed., Pierce Chem. Co.: Rockford, Ill., 1984; Kent, Ann. Rev. Biochem. 57:957-89, 1988; and Gregg, et al., Int. J. Peptide Protein Res. 55:161-214, 1990, which are incorporated herein by reference in their entirety.)

Peptides of the present invention may be prepared by solid phase methodology. Briefly, an N-protected C-terminal amino acid residue is linked to an insoluble support such as divinylbenzene cross-linked polystyrene, polyacrylamide resin, Kieselguhr/polyamide (pepsyn K), controlled pore glass, cellulose, polypropylene membranes, acrylic acid-coated polyethylene rods, or the like. Cycles of deprotection, neutralization, and coupling of successive protected amino acid derivatives are used to link the amino acids from the C-terminus according to the amino acid sequence. For some synthetic peptides, an FMOC strategy using an acid-sensitive resin may be used. Solid supports in this regard may be divinylbenzene cross-linked polystyrene resins, which are commercially available in a variety of functionalized forms, including chloromethyl resin, hydroxymethyl resin, paraacetamidomethyl resin, benzhydrylamine (BHA) resin, 4-methylbenzhydrylamine (MBHA) resin, oxime resins, 4-alkoxybenzyl alcohol resin (Wang resin), 4-(2′,4′-dimethoxyphenylaminomethyl)-phenoxymethyl resin, 2,4-dimethoxybenzhydryl-amine resin, and 4-(2′,4′-dimethoxyphenyl-FMOC-amino-methyl)-phenoxyacetamidonorleucyl-MBHA resin (Rink amide MBHA resin). In addition, acid-sensitive resins also provide C-terminal acids, if desired. A protecting group for alpha amino acids is base-labile 9-fluorenylmethoxy-carbonyl (FMOC).

Suitable protecting groups for the side chain functionalities of amino acids chemically compatible with BOC (t-butyloxycarbonyl) and FMOC groups are well known in the art. When using FMOC chemistry, the following protected amino acid derivatives may be utilized: FMOC-Cys(Trt), FMOC-Ser(But), FMOC-Asn(Trt), FMOC-Leu, FMOC-Thr(Trt), FMOC-Val, FMOC-Gly, FMOC-Lys(Boc), FMOC-Gln(Trt), FMOC-Glu(OBut), FMOC-His(Trt), FMOC-Tyr(But), FMOC-Arg(PMC (2,2,5,7,8-pentamethylchroman-6-sulfonyl)), FMOC-Arg(BOC)₂, FMOC-Pro, and FMOC-Trp(BOC). The amino acid residues may be coupled by using a variety of coupling agents and chemistries known in the art, such as direct coupling with DIC (diisopropyl-carbodiimide), DCC (dicyclohexylcarbodiimide), BOP (benzotriazolyl-N-oxytrisdimethylaminophosphonium hexafluorophosphate), PyBOP (benzotriazole-1-yl-oxy-tris-pyrrolidinophosphonium hexafluorophosphate), PyBrOP (bromo-tris-pyrrolidinophosphonium hexafluorophosphate); via performed symmetrical anhydrides; via active esters such as pentafluorophenyl esters; or via performed HOBt (1-hydroxybenzotriazole) active esters or by using FMOC-amino acid fluoride and chlorides or by using FMOC-amino acid-N-carboxy anhydrides. For example, activation may be performed with HBTU (2-(1H-benzotriazole-1-yl), 1,1,3,3-tetramethyluronium hexafluorophosphate) or HATU (2-(1H-7-aza-benzotriazole-1-yl),1,1,3,3-tetramethyluronium hexafluoro-phosphate) in the presence of HOBt or HOAt (7-azahydroxybenztriazole).

The solid phase method may be carried out manually, or by automated synthesis on a commercially available peptide synthesizer (e.g., Applied Biosystems 431A or the like; Applied Biosystems, Foster City, Calif.). In a typical synthesis, the first (C-terminal) amino acid is loaded on the chlorotrityl resin. Successive deprotection (with 20% piperidine/NMP (N-methylpyrrolidone)) and coupling cycles according to ABI FastMoc protocols (Applied Biosystems) may be used to generate the peptide sequence. Double and triple coupling, with capping by acetic anhydride, may also be used.

The synthetic mimetic peptide may be cleaved from the resin and deprotected by treatment with TFA (trifluoroacetic acid) containing appropriate scavengers. Many such cleavage reagents, such as Reagent K (0.75 g crystalline phenol, 0.25 mL ethanedithiol, 0.5 mL thioanisole, 0.5 mL deionized water, 10 mL TFA) and others, may be used. The peptide is separated from the resin by filtration and isolated by ether precipitation. Further purification may be achieved by conventional methods, such as gel filtration and reverse phase HPLC (high performance liquid chromatography). Synthetic mimetics according to the present invention may be in the form of pharmaceutically acceptable salts, especially base-addition salts including salts of organic bases and inorganic bases. The base-addition salts of the acidic amino acid residues are prepared by treatment of the peptide with the appropriate base or inorganic base, according to procedures well known to those skilled in the art, or the desired salt may be obtained directly by lyophilization of the appropriate base.

Generally, those skilled in the art will recognize that peptides as described herein may be modified by a variety of chemical techniques to produce peptides having essentially the same activity as the unmodified peptide, and optionally having other desirable properties. For example, carboxylic acid groups of the peptide may be provided in the form of a salt of a pharmaceutically-acceptable cation. Amino groups within the peptide may be in the form of a pharmaceutically-acceptable acid addition salt, such as the HCl, HBr, acetic, benzoic, toluene sulfonic, maleic, tartaric, and other organic salts, or may be converted to an amide. Thiols may be protected with any one of a number of well-recognized protecting groups, such as acetamide groups. Those skilled in the art will also recognize methods for introducing cyclic structures into the peptides of this invention so that the native binding configuration will be more nearly approximated. For example, a carboxyl terminal or amino terminal cysteine residue may be added to the peptide, so that when oxidized the peptide will contain a disulfide bond, thereby generating a cyclic peptide. Other peptide cyclizing methods include the formation of thioethers and carboxyl- and amino-terminal amides and esters.

Specifically, a variety of techniques are available for constructing peptide derivatives and analogs with the same or similar desired biological activity as the corresponding peptide but with more favorable activity than the peptide with respect to solubility, stability, and susceptibility to hydrolysis and proteolysis. Such derivatives and analogs include peptides modified at the N-terminal amino group, as exemplified by, but not limited to, the peptides of Formula (I) (e.g., Table 1), the C-terminal amide group, and/or changing one or more of the amido linkages in the peptide to a non-amido linkage. It will be understood that two or more such modifications may be coupled in one peptide mimetic structure (e.g., modification at the C-terminal amide group and inclusion of a —CH₂— carbamate linkage between two amino acids in the peptide).

Peptide mimetics as understood in the art and provided by the invention are structurally similar to the peptides of the invention, but have one or more peptide linkages optionally replaced by a linkage, for example, —CH₂NH—, —CH₂S—, —CH₂CH₂—, —CH═CH— (in both cis and trans conformers), —COCH₂—, —CH(OH)CH₂—, and —CH₂SO—, by methods known in the art and further described in the following references: Spatola, Chemistry and Biochemistry of Amino Acids, Peptides, and Proteins, (Weinstein, ed.), Marcel Dekker: New York, p. 267, 1983; Spatola, Peptide Backbone Modifications 1:3, 1983; Morley, Trends Pharm. Sci. pp. 463-468, 1980; Hudson, et al., Int. J. Pept. Prot. Res. 14:177-185, 1979; Spatola, et al., Life Sci. 38:1243-1249, 1986; Hann, J. Chem. Soc. Perkin Trans. I 307-314, 1982; Almquist, et al., J. Med. Chem. 23:1392-1398, 1980; Jennings-White, et al., Tetrahedron Lett. 23:2533, 1982; Szelke, et al., EP045665A; Holladay, et al., Tetrahedron Lett. 24:4401-4404, 1983; and Hruby, Life Sci. 31:189-199, 1982; each of which is incorporated herein by reference. Such peptide mimetics may have significant advantages over peptide embodiments, including, for example, more economical to produce, having greater chemical stability or enhanced pharmacological properties (such as half-life, absorption, potency, efficacy, etc.), reduced antigenicity, and other properties.

Mimetic analogs of the peptides of the invention may also be obtained using the principles of conventional or rational drug design (see, e.g., Andrews, et al., Proc. Alfred Benzon Symp. 28:145-165, 1990; McPherson, Eur. J. Biochem. 189:1-24, 1990; Hol, et al., in Molecular Recognition: Chemical and Biochemical Problems, (Roberts, ed.); Royal Society of Chemistry; pp. 84-93, 1989a; Hol, Arzneim-Forsch. 39:1016-1018, 1989b; Hol, Agnew Chem. Int. Ed. Engl. 25:767-778, 1986; the disclosures of which are herein incorporated by reference).

In accordance with the methods of conventional drug design, the desired mimetic molecules may be obtained by randomly testing molecules whose structures have an attribute in common with the structure of a “native” peptide. The quantitative contribution that results from a change in a particular group of a binding molecule may be determined by measuring the biological activity of the putative mimetic in comparison with the activity of the peptide. In one embodiment of rational drug design, the mimetic is designed to share an attribute of the most stable three-dimensional conformation of the peptide. Thus, for example, the mimetic may be designed to possess chemical groups that are oriented in a way sufficient to cause ionic, hydrophobic, or van der Waals interactions that are similar to those exhibited by the peptides of the invention, as disclosed herein.

One method for performing rational mimetic design employs a computer system capable of forming a representation of the three-dimensional structure of the peptide, such as those exemplified by Hol, 1989a; Hol, 1989b; and Hol, 1986. Molecular structures of the peptido-, organo-, and chemical mimetics of the peptides of the invention may be produced using computer-assisted design programs commercially available in the art. Examples of such programs include SYBYL 6.5®, HQSAR™, and ALCHEMY 2000™ (Tripos); GALAXY™ and AM2000™ (AM Technologies, Inc., San Antonio, Tex.); CATALYST™ and CERIUS™ (Molecular Simulations, Inc., San Diego, Calif.); CACHE PRODUCTS™, TSAR™, AMBER™, and CHEM-X™ (Oxford Molecular Products, Oxford, Calif.) and CHEMBUILDER3D™ (Interactive Simulations, Inc., San Diego, Calif.).

The peptido-, organs, and chemical mimetics produced using the peptides disclosed herein using, for example, art-recognized molecular modeling programs may be produced using conventional chemical synthetic techniques, for example, designed to accommodate high throughput screening, including combinatorial chemistry methods. Combinatorial methods useful in the production of the peptido-, organs, and chemical mimetics of the invention include phage display arrays, solid-phase synthesis, and combinatorial chemistry arrays, as provided, for example, by SIDDCO (Tuscon, Ariz.); Tripos, Inc.; Calbiochem/Novabiochem (San Diego, Calif.); Symyx Technologies, Inc. (Santa Clara, Calif.); Medichem Research, Inc. (Lemont, Ill.); Pharm-Eco Laboratories, Inc. (Bethlehem, Pa.); or N.V. Organon (Oss, Netherlands). Combinatorial chemistry production of the peptido-, organo-, and chemical mimetics of the invention may be produced according to methods known in the art, including, but not limited to, techniques disclosed in Terrett, (Combinatorial Chemistry, Oxford University Press, London, 1998); Gallop, et al., J. Med. Chem. 37:1233-51, 1994; Gordon, et al., J. Med. Chem. 37:1385-1401, 1994; Look, et al., Bioorg. Med. Chem. Lett. 6:707-12, 1996; Ruhland, et al., J. Amer. Chem. Soc. 118: 253-4, 1996; Gordon, et al., Acc. Chem. Res. 29:144-54, 1996; Thompson & Ellman, Chem. Rev. 96:555-600, 1996; Fruchtel & Jung, Angew. Chem. Int. Ed. Engl. 35:17-42, 1996; Pavia, “The Chemical Generation of Molecular Diversity”, Network Science Center, www.netsci.org, 1995; Adnan, et al., “Solid Support Combinatorial Chemistry in Lead Discovery and SAR Optimization,” Id., 1995; Davies and Briant, “Combinatorial Chemistry Library Design using Pharmacophore Diversity,” Id., 1995; Pavia, “Chemically Generated Screening Libraries: Present and Future,” Id., 1996; and U.S. Pat. Nos. 5,880,972; 5,463,564; 5,331573; and 5,573,905.

The newly synthesized peptides may be substantially purified by preparative high performance liquid chromatography (see, e.g., Creighton, Proteins: Structures And Molecular Principles, WH Freeman and Co., New York, N.Y., 1983). The composition of a synthetic peptide of the present invention may be confirmed by amino acid analysis or sequencing by, for example, the Edman degradation procedure (Creighton, supra). Additionally, any portion of the amino acid sequence of the peptide may be altered during direct synthesis and/or combined using chemical methods with sequences from other proteins to produce a variant peptide or a fusion peptide.

Also included in this invention are antibodies and antibody fragments that selectively bind the peptides of this invention. Any type of antibody known in the art may be generated using methods well known in the art. For example, an antibody may be generated to bind specifically to an epitope of a peptide of this invention. “Antibody” as used herein includes intact immunoglobulin molecules, as well as fragments thereof, such as Fab, F(ab′)₂, and Fv, which are capable of binding an epitope of a peptide of this invention. Typically, 6, 8, 10, or 12 contiguous amino acids are required to form an epitope. However, epitopes which involve non-contiguous amino acids may require more amino acids, for example, 15, 25, or 50 amino acids.

An antibody which specifically binds to an epitope of a peptide of this invention may be used therapeutically, as well as in immunochemical assays, such as Western blots, ELISAs, radioimmunoassays, immunohistochemical assays, immunoprecipitations, or other immunochemical assays known in the art. Various immunoassays may be used to identify antibodies having the desired specificity. Numerous protocols for competitive binding or Immunoradiometric assays are well known in the art. Such immunoassays typically involve the measurement of complex formation between an immunogen and an antibody which specifically binds to the immunogen.

Typically, an antibody which specifically binds to a peptide of this invention provides a detection signal higher than a detection signal provided with other proteins when used in an immunochemical assay. Antibodies which specifically bind to a peptide of this invention do not detect other proteins in immunochemical assays and can immunoprecipitate a peptide of this invention from solution.

Peptides of this invention may be used to immunize a mammal, such as a mouse, rat, rabbit, guinea pig, goat, sheep, monkey, or human, to produce polyclonal antibodies. If desired, a peptide of this invention may be conjugated to a carrier protein, such as bovine serum albumin, thyroglobulin, and keyhole limpet hemocyanin. Depending on the host species, various adjuvants may be used to increase the immunological response. Such adjuvants include, but are not limited to, Freund's adjuvant, mineral gels (e.g., aluminum hydroxide), and surface active substances (e.g., lysolecithin, pluronic polyols, polyanions, peptides, oil emulsions, keyhole limpet hemocyanin, and dinitrophenol). Among adjuvants used in humans, BCG (bacilli Calmette-Guerin) and Corynebacterium parvum are especially useful.

Monoclonal antibodies which specifically bind to a peptide of this invention may be prepared using any technique which provides for the production of antibody molecules by continuous cell lines in culture. These techniques include, but are not limited to, the hybridoma technique, the human B cell hybridoma technique, and the EBV hybridoma technique (Kohler, et al., Nature 256:495-97, 1985; Kozbor, et al., J. Immunol. Methods 81:3142, 1985; Cote, et al., Proc. Natl. Acad. Sci. 80:2026-30, 1983; Cole, et al., Mol. Cell. Biol. 62:109-20, 1984).

In addition, techniques developed for the production of “chimeric antibodies,” the splicing of mouse antibody genes to human antibody genes to obtain a molecule with appropriate antigen specificity and biological activity, may be used (Morrison, et al., Proc. Natl. Acad. Sci. 81:6851-55, 1984; Neuberger, et al., Nature 312:604-08, 1984; Takeda, et al., Nature 314:452-54, 1985). Monoclonal and other antibodies also can be “humanized” to prevent a patient from mounting an immune response against the antibody when it is used therapeutically. Such antibodies may be sufficiently similar in sequence to human antibodies to be used directly in therapy or may require alteration of a few key residues. Sequence differences between rodent antibodies and human sequences may be minimized by replacing residues which differ from those in the human sequences by site directed mutagenesis of individual residues or by grafting of entire complementarity determining regions. Alternatively, humanized antibodies may be produced using recombinant methods (see, e.g., GB2188638B). Antibodies which specifically bind to a peptide of this invention may contain antigen binding sites which are either partially or fully humanized, as disclosed in U.S. Pat. No. 5,565,332.

Alternatively, techniques described for the production of single chain antibodies may be adapted using methods known in the art to produce single chain antibodies which specifically bind to a peptide of this invention. Antibodies with related specificity, but of distinct idiotypic composition, can be generated by chain shuffling from random combinatorial immunoglobin libraries (Burton, Proc. Natl. Acad. Sci. 88:11120-23, 1991).

Single-chain antibodies also may be constructed using a DNA amplification method, such as PCR, using hybridoma cDNA as a template (Thirion, et al., Eur. J. Cancer Prev. 5:507-11, 1996). Single-chain antibodies can be mono- or bispecific, and can be bivalent or tetravalent. Construction of tetravalent, bispecific single-chain antibodies is taught, for example, in Coloma & Morrison (Nat. Biotechnol. 15:159-63, 1997). Construction of bivalent, bispecific single-chain antibodies is taught in Mallender & Voss (J. Biol. Chem. 269:199-206, 1994).

A nucleotide sequence encoding a single-chain antibody may be constructed using manual or automated nucleotide synthesis, cloned into an expression construct using standard recombinant DNA methods, and introduced into a cell to express the coding sequence, as described below. Alternatively, single-chain antibodies can be produced directly using, for example, filamentous phage technology (Verhaar, et al., Int. J. Cancer 61:497-501, 1995; Nicholls, et al., J. Immunol. Meth. 165:81-91, 1993).

Antibodies which specifically bind to a peptide of this invention may also be produced by inducing in vivo production in the lymphocyte population or by screening immunoglobulin libraries or panels of highly specific binding reagents as disclosed in the literature (Orlandi, et al., Proc. Natl. Acad. Sci. 86:38333-37, 1989; Winter, et al., Nature 349:293-99, 1991).

Other types of antibodies may be constructed and used therapeutically in methods of the invention. For example, chimeric antibodies may be constructed as disclosed in WO 93/03151. Binding proteins which are derived from immunoglobulins and which are multivalent and multispecific, such as the “diabodies” also can be prepared (see, e.g., WO 94/13804,).

Human antibodies with the ability to bind to the peptides of this invention may also be identified from the MorphoSys HuCAL® library as follows. A peptide of this invention may be coated on a microtiter plate and incubated with the MorphoSys HuCAL® Fab phage library. Those phage-linked Fabs not binding to the peptide of this invention can be washed away from the plate, leaving only phage which tightly bind to the peptide of this invention. The bound phage can be eluted, for example, by a change in pH or by elution with E. coli and amplified by infection of E. coli hosts. This panning process can be repeated once or twice to enrich for a population of antibodies that tightly bind to the peptide of this invention. The Fabs from the enriched pool are then expressed, purified, and screened in an ELISA assay.

Antibodies according to the invention may be purified by methods well known in the art. For example, antibodies may be affinity purified by passage over a column to which a peptide of this invention is bound. The bound antibodies can then be eluted from the column using a buffer with a high salt concentration.

### Methods of Use

As used herein, various terms are defined below.

When introducing elements of the present invention or the preferred embodiment(s) thereof, the articles “a,” “an,” “the,” and “said” are intended to mean that there are one or more of the elements. The terms “comprising,” “including,” and “having” are intended to be inclusive and mean that there may be additional elements other than the listed elements.

The term “subject” as used herein includes mammals (e.g., humans and animals).

The term “treatment” includes any process, action, application, therapy, or the like, wherein a subject, including a human being, is provided medical aid with the object of improving the subject's condition, directly or indirectly, or slowing the progression of a condition or disorder in the subject.

The term “combination therapy” or “co-therapy” means the administration of two or more therapeutic agents to treat, for example, diabetes. Such administration encompasses co-administration of two or more therapeutic agents in a substantially simultaneous manner, such as in a single capsule having a fixed ratio of active ingredients or in multiple, separate capsules for each inhibitor agent. In addition, such administration encompasses use of each type of therapeutic agent in a sequential manner.

The phrase “therapeutically effective” means the amount of each agent administered that will achieve the goal of improvement in a diabetic condition or disorder severity, while avoiding or minimizing adverse side effects associated with the given therapeutic treatment.

The term “pharmaceutically acceptable” means that the subject item is appropriate for use in a pharmaceutical product.

The peptides of Formula (I) are expected to be valuable as therapeutic agents (e.g., Table 1). Accordingly, an embodiment of this invention includes a method of treating the various conditions in a patient (including mammals) which comprises administering to said patient a composition containing an amount of the peptide of Formula (I), that is effective in treating the target condition.

The peptides of the present invention, as a result of the ability to stimulate insulin secretion from pancreatic islet cells in vitro, and by causing a decrease in blood glucose in vivo, may be employed in treatment diabetes, including both type 1 and type 2 diabetes (non-insulin dependent diabetes mellitus). Such treatment may also delay the onset of diabetes and diabetic complications. The peptides may be used to prevent subjects with impaired glucose tolerance from proceeding to develop type 2 diabetes. Other diseases and conditions that may be treated or prevented using compounds of the invention in methods of the invention include: Maturity-Onset Diabetes of the Young (MODY) (Herman, et al., Diabetes 43:40, 1994); Latent Autoimmune Diabetes Adult (LADA) (Zimmet, et al., Diabetes Med. 11:299, 1994); impaired glucose tolerance (IGT) (Expert Committee on Classification of Diabetes Mellitus, Diabetes Care 22 (Supp. 1):S5, 1999); Impaired fasting glucose (IFG) (Charles, et al., Diabetes 40:796, 1991); gestational diabetes (Metzger, Diabetes, 40:197, 1991); and metabolic syndrome X.

The peptides of the present invention may also be utilized in the prevention and/or treatment of obesity (e.g., regulation of appetite and food intake); disorders of energy homeostasis; disorders of lipid and carbohydrate metabolism; cardiovascular disease, including atherosclerosis, coronary heart disease, coronary artery disease, hyperlipidemia, hypercholesteremia, low HDL levels and hypertension; cerebrovascular disease and peripheral vessel disease; polycystic ovary syndrome; carcinogenesis, and hyperplasia; asthma and chronic obstructive pulmonary disease; male reproduction problems (including erectile dysfunction); ulcers; neurodegenerative diseases (including Parkinson's and Alzheimer's); sleep disorders and circadian dysfunction; growth disorders; immune diseases, including autoimmune diseases (e.g., systemic lupus erythematosus); chronic inflammatory diseases; septic shock; HIV infection and AIDS, and other conditions identified herein, or function otherwise as described later herein.

The compounds of the present invention may also be useful for treating physiological disorders related to, for example, cell differentiation to produce lipid accumulating cells, regulation of insulin sensitivity and blood glucose levels, which are involved in, for example, abnormal pancreatic β-cell function, macrophage differentiation which leads to the formation of atherosclerotic plaques, inflammatory response, carcinogenesis, hyperplasia, reduction in the pancreatic β-cell mass, insulin secretion, tissue sensitivity to insulin, liposarcoma cell growth, polycystic ovarian disease, chronic anovulation, hyperandrogenism, progesterone production, steroidogenesis, redox potential and oxidative stress in cells, nitric oxide synthase (NOS) production, increased gamma glutamyl transpeptidase, catalase, plasma triglycerides, HDL, and LDL cholesterol levels, and the like.

Compounds of the invention may also be used in methods of the invention to treat secondary causes of diabetes (Expert Committee on Classification of Diabetes Mellitus, Diabetes Care 22 (Supp. 1):S5, 1999). Such secondary causes include glucocorticoid excess, growth hormone excess, pheochromocytoma, and drug-induced diabetes. Drugs that may induce diabetes include, but are not limited to, pyriminil, nicotinic acid, glucocorticoids, phenyloin, thyroid hormone, β-adrenergic agents, α-interferon and drugs used to treat HIV infection.

In addition, the peptides of the invention may be used for treatment of asthma (Bolin, et al., Biopolymer 37:57-66, 1995; U.S. Pat. No. 5,677,419; showing that peptide R3P0 is active in relaxing guinea pig tracheal smooth muscle); hypotension induction (VIP induces hypotension, tachycardia, and facial flushing in asthmatic patients (Morice, et al., Peptides 7:279-280, 1986; Morice, et al., Lancet 2:1225-1227, 1983); male reproduction problems (Siow, et al., Arch. Androl. 43(1):67-71, 1999); as an anti-apoptosis/neuroprotective agent (Brenneman, et al., Ann. N.Y. Acad. Sci. 865:207-12, 1998); cardioprotection during ischemic events (Kalfin, et al., J. Pharmacol. Exp. Ther. 1268(2):952-8, 1994; Das, et al., Ann. N.Y. Acad. Sci. 865:297-308, 1998), manipulation of the circadian clock and its associated disorders (Hamar, et al., Cell 109:497-508, 2002; Shen, et al., Proc. Natl. Acad. Sci. 97:11575-80, 2000), and finally as an anti-ulcer agent (Tuncel, et al., Ann. N.Y. Acad. Sci. 865:309-22, 1998).

The peptides of the present invention may be used alone or in combination with additional therapies and/or compounds known to those skilled in the art in the treatment of diabetes and related disorders. Alternatively, the methods and compounds described herein may be used, partially or completely, in combination therapy.

The peptides of the invention may also be administered in combination with other known therapies for the treatment of diabetes, including PPAR ligands (e.g., agonists, antagonists), insulin secretagogues, for example, sulfonylurea drugs and non-sulfonylurea secretagogues, α-glucosidase inhibitors, insulin sensitizers, insulin secretagogues, hepatic glucose output lowering compounds, Insulin and insulin derivatives, and anti-obesity drugs. Such therapies may be administered prior to, concurrently with, or following administration of the compounds of the invention. Insulin and insulin derivatives include both long and short acting forms and formulations of insulin. PPAR ligands may include agonists and/or antagonists of any of the PPAR receptors or combinations thereof. For example, PPAR ligands may include ligands of PPAR-α, PPAR-γ, PPAR-δ or any combination of two or three of the receptors of PPAR. PPAR ligands include, for example, rosiglitazone, troglitazone, and pioglitazone. Sulfonylurea drugs include, for example, glyburide, glimepiride, chlorpropamide, tolbutamide, and glipizide. α-glucosidase inhibitors that may be useful in treating diabetes when administered with a compound of the invention include acarbose, miglitol, and voglibose. Insulin sensitizers that may be useful in treating diabetes include PPAR-γ agonists such as the glitazones (e.g., troglitazone, pioglitazone, englitazone, MCC-555, rosiglitazone, and the like) and other thiazolidinedione and non-thiazolidinedione compounds; biguanides such as metformin and phenformin; protein tyrosine phosphatase-1B (PTP-1B) Inhibitors; dipeptidyl peptidase IV (DPPIV) inhibitors; and 11beta-HSD Inhibitors. Hepatic glucose output lowering compounds that may be useful in treating diabetes when administered with a peptide of the invention include, for example, glucagon anatgonists and metformin, such as Glucophage and Glucophage XR. Insulin secretagogues that may be useful in treating diabetes when administered with a peptide of the invention include sulfonylurea and non-sulfonylurea drugs: GLP-1, GIP, VIP, PACAP, secretin, and derivatives thereof; nateglinide, meglitinide, repaglinide, glibenclamide, glimepiride, chlorpropamide, and glipizide. For example, GLP-1 includes derivatives of GLP-1 with longer half-lives than native GLP-1, such as, for example, fatty-acid derivatized GLP-1 and exendin. In one embodiment of the invention, peptides of the invention are used in combination with insulin secretagogues to increase the sensitivity of pancreatic β-cells to the insulin secretagogue.

Peptides of the invention may also be used in methods of the invention in combination with anti-obesity drugs. For example, anti-obesity drugs include β-3 adrenergic receptor agonists such as CL 316,243; cannabinoid (e.g., CB-1) antagonists such as Rimonabant; neuropeptide-Y receptor antagonists; neuropeptide Y5 inhibitors; apo-B/MTP inhibitors; 11β-hydroxy steroid dehydrogenase-1 inhibitors; peptide YY₃₋₃₆ or analogs thereof; MCR4 agonists; CCK-A agonists; monoamine reuptake inhibitors; sympathomimetic agents; dopamine agonists; melanocyte-stimulating hormone receptor analogs; melanin concentrating hormone antagonists; leptin; leptin analogs; leptin receptor agonists; galanin antagonists; lipase inhibitors; bombesin agonists; thyromimetic agents; dehydroepiandrosterone or analogs thereof; glucocorticoid receptor antagonists; orexin receptor antagonists; ciliary neurotrophic factor; ghrelin receptor antagonists; histamine-3 receptor antagonists; neuromedin U receptor agonists; appetite suppressants, such as, for example, sibutramine (Meridia); and lipase inhibitors, such as, for example, orlistat (Xenical). The compounds of the present invention may also be administered in combination with a drug compound that modulates digestion and/or metabolism such as drugs that modulate thermogenesis, lipolysis, gut motility, fat absorption, and satiety.

Peptides of the invention may also be used in methods of the invention in combination with drugs commonly used to treat lipid disorders. Such drugs include, but are not limited to, HMG-CoA reductase inhibitors, nicotinic acid, fatty acid lowering compounds (e.g., acipimox); lipid lowering drugs (e.g., stanol esters, sterol glycosides such as tiqueside, and azetidinones such as ezetimibe), ACAT inhibitors (such as avasimibe), bile acid sequestrants, bile acid reuptake inhibitors, microsomal triglyceride transport inhibitors, and fibric acid derivatives. HMG-CoA reductase Inhibitors include, for example, lovastatin, simvastatin, pravastatin, fluvastatin, atorvastatin, rivastatin, itavastatin, cerivastatin, and ZD-4522. Fibric acid derivatives include, for example, clofibrate, fenofibrate, bezafibrate, ciprofibrate, beclofibrate, etofibrate, and gemfibrozil. Sequestrants include, for example, cholestyramine, colestipol, and dialkylaminoalkyl derivatives of a cross-linked dextran.

Furthermore, peptides of the invention may also be administered in combination with anti-hypertensive drugs, such as, for example, β-blockers and ACE inhibitors. Examples of additional ant-hypertensive agents for use in combination with the peptides of the present invention include calcium channel blockers (L-type and T-type; e.g., diltiazem, verapamil, nifedipine, amlodipine and mybefradil), diuretics (e.g., chlorothiazide, hydrochlorothiazide, flumethiazide, hydroflumethiazide, bendroflumethiazide, methylchlorothiazide, trichloromethiazide, polythiazide, benzthiazide, ethacrynic acid tricrynafen, chlorthalidone, furosemide, musolimine, bumetanide, triamtrenene, amiloride, spironolactone), renin inhibitors, ACE inhibitors (e.g., captopril, zofenopril, fosinopril, enalapril, ceranopril, cilazopril, delapril, pentopril, quinapril, ramipril, lisinopril), AT-1 receptor antagonists (e.g., losartan, irbesartan, valsartan), ET receptor antagonists (e.g., sitaxsentan, atrsentan, neutral endopeptidase (NEP) inhibitors, vasopepsidase inhibitors (dual NEP-ACE inhibitors) (e.g., omapatrilat and gemopatrilat), and nitrates.

Such co-therapies may be administered in any combination of two or more drugs (e.g., a compound of the invention in combination with an insulin sensitizer and an anti-obesity drug). Such co-therapies may be administered in the form of pharmaceutical compositions, as described above.

### Pharmaceutical Compositions

Based on well known assays used to determine the efficacy for treatment of conditions identified above in mammals, and by comparison of these results with the results of known medicaments that are used to treat these conditions, the effective dosage of the peptides of this invention can readily be determined for treatment of each desired indication. The amount of the active ingredient to be administered in the treatment of one of these conditions can vary widely according to such considerations as the particular peptide and dosage unit employed, the mode of administration, the period of treatment, the age and sex of the patient treated, and the nature and extent of the condition treated.

The total amount of the active ingredient to be administered may generally range from, for example, about 0.0001 mg/kg to about 200 mg/kg, or from about 0.001 mg/kg to about 200 mg/kg body weight per day. A unit dosage may contain from, for example, about 0.05 mg to about 1500 mg of active ingredient, and may be administered one or more times per day. The daily dosage for administration by injection, including intravenous, intramuscular, subcutaneous, and parenteral injections, and use of infusion techniques may be from, for example, about 0.001 to about 200 mg/kg. The daily rectal dosage regimen may be from, for example, about 0.001 to about 200 mg/kg of total body weight. The transdermal concentration may be that required to maintain a daily dose of from, for example, about 0.001 to about 200 mg/kg.

Of course, the specific initial and continuing dosage regimen for each patient will vary according to the nature and severity of the condition as determined by the attending diagnostician, the activity of the specific peptide employed, the age of the patient, the diet of the patient, time of administration, route of administration, rate of excretion of the drug, drug combinations, and the like. The desired mode of treatment and number of doses of a peptide of the present invention may be ascertained by those skilled in the art using conventional treatment tests.

The peptides of this invention may be utilized to achieve the desired pharmacological effect by administration to a subject in need thereof in an appropriately formulated pharmaceutical composition. A subject, for example, may be a mammal, including a human, in need of treatment for a particular condition or disease. Therefore, the present invention includes pharmaceutical compositions which are comprised of a pharmaceutically acceptable carrier and a pharmaceutically effective amount of a peptide of the present invention. A pharmaceutically acceptable carrier is any carrier which is relatively non-toxic and innocuous to a patient at concentrations consistent with effective activity of the active ingredient so that any side effects ascribable to the carrier do not vitiate the beneficial effects of the active ingredient. A pharmaceutically effective amount of a peptide is that amount which produces a result or exerts an influence on the particular condition being treated. The peptides of the present invention may be administered with a pharmaceutically-acceptable carrier using any effective conventional dosage unit forms, including, for example, immediate and timed release preparations, orally, parenterally, topically, or the like.

For oral administration, the peptides may be formulated into solid or liquid preparations such as, for example, capsules, pills, tablets, troches, lozenges, melts, powders, solutions, suspensions, or emulsions, and may be prepared according to methods known to the art for the manufacture of pharmaceutical compositions. The solid unit dosage forms may be a capsule which can be of the ordinary hard- or soft-shelled gelatin type containing, for example, surfactants, lubricants, and inert fillers such as lactose, sucrose, calcium phosphate, and corn starch.

In another embodiment, the peptides of this invention may be tableted with conventional tablet bases such as lactose, sucrose, and cornstarch in combination with binders such as acacia, cornstarch, or gelatin; disintegrating agents intended to assist the break-up and dissolution of the tablet following administration such as potato starch, alginic acid, corn starch, and guar gum; lubricants intended to improve the flow of tablet granulation and to prevent the adhesion of tablet material to the surfaces of the tablet dies and punches, for example, talc, stearic acid, or magnesium, calcium or zinc stearate; dyes; coloring agents; and flavoring agents intended to enhance the aesthetic qualities of the tablets and make them more acceptable to the patent. Suitable excipients for use in oral liquid dosage forms include diluents such as water and alcohols, for example, ethanol, benzyl alcohol, and polyethylene alcohols, either with or without the addition of a pharmaceutically acceptable surfactant, suspending agent, or emulsifying agent. Various other materials may be present as coatings or to otherwise modify the physical form of the dosage unit. For instance tablets, pills or capsules may be coated with shellac, sugar or both.

Dispersible powders and granules are suitable for the preparation of an aqueous suspension. They provide the active ingredient in admixture with a dispersing or wetting agent, a suspending agent, and one or more preservatives. Suitable dispersing or wetting agents and suspending agents are exemplified by those already mentioned above. Additional excipients, for example, those sweetening, flavoring and coloring agents described above, may also be present.

The pharmaceutical compositions of this invention may also be in the form of oil-in-water emulsions. The oily phase may be a vegetable oil such as liquid paraffin or a mixture of vegetable oils. Suitable emulsifying agents may be (1) naturally occurring gums such as gum acacia and gum tragacanth, (2) naturally occurring phosphatides such as soy bean and lecithin, (3) esters or partial esters derived from fatty acids and hexitol anhydrides, for example, sorbitan monooleate, and (4) condensation products of said partial esters with ethylene oxide, for example, polyoxyethylene sorbitan monooleate. The emulsions may also contain sweetening and flavoring agents.

Syrups and elixirs may be formulated with sweetening agents such as, for example, glycerol, propylene glycol, sorbitol, or sucrose. Such formulations may also contain a demulcent, and preservative, flavoring and coloring agents.

The peptides of this invention may also be administered parenterally, that is, subcutaneously, intravenously, intramuscularly, or interperitoneally, as injectable dosages of the peptide in a physiologically acceptable diluent with a pharmaceutical carrier which may be a sterile liquid or mixture of liquids such as water, saline, aqueous dextrose and related sugar solutions; an alcohol such as ethanol, isopropanol, or hexadecyl alcohol; glycols such as propylene glycol or polyethylene glycol; glycerol ketals such as 2,2-dimethyl-1,1-dioxolane-4-methanol, ethers such as poly(ethyleneglycol) 400; an oil; a fatty acid; a fatty acid ester or glyceride; or an acetylated fatty acid glyceride with or without the addition of a pharmaceutically acceptable surfactant such as a soap or a detergent, suspending agent such as pectin, carbomers, methycellulose, hydroxypropylmethylcellulose, or carboxymethylcellulose, or emulsifying agent and other pharmaceutical adjuvants.

Illustrative of oils which can be used in the parenteral formulations of this invention are those of petroleum, animal, vegetable, or synthetic origin, for example, peanut oil, soybean oil, sesame oil, cottonseed oil, corn oil, olive oil, petrolatum, and mineral oil. Suitable fatty acids include oleic acid, stearic acid, and Isostearic acid. Suitable fatty acid esters are, for example, ethyl oleate and isopropyl myristate. Suitable soaps include fatty alkali metal, ammonium, and triethanolamine salts and suitable detergents include cationic detergents, for example, dimethyl dialkyl ammonium halides, alkyl pyridinium halides, and alkylamine acetates; anionic detergents, for example, alkyl, aryl, and olefin sulfonates, alkyl, olefin, ether, and monoglyceride sulfates, and sulfosuccinates; nonionic detergents, for example, fatty amine oxides, fatty acid alkanolamides, and polyoxyethylenepolypropylene copolymers; and amphoteric detergents, for example, alkyl-beta-aminopropionates, and 2-alkylimidazoline quarternary ammonium salts, as well as mixtures.

The parenteral compositions of this invention may typically contain from about 0.5% to about 25% by weight of the active ingredient in solution. Preservatives and buffers may also be used advantageously. In order to minimize or eliminate irritation at the site of injection, such compositions may contain a non-ionic surfactant having a hydrophile-lipophile balance (HLB) of from about 12 to about 17. The quantity of surfactant in such formulation ranges from about 5% to about 15% by weight. The surfactant can be a single component having the above HLB or can be a mixture of two or more components having the desired HLB.

Illustrative of surfactants used in parenteral formulations are the class of polyethylene sorbitan fatty acid esters, for example, sorbitan monooleate and the high molecular weight adducts of ethylene oxide with a hydrophobic base, formed by the condensation of propylene oxide with propylene glycol.

The pharmaceutical compositions may be in the form of sterile injectable aqueous suspensions. Such suspensions may be formulated according to known methods using suitable dispersing or wetting agents and suspending agents such as, for example, sodium carboxymethylcellulose, methylcellulose, hydroxypropylmethyl-cellulose, sodium alginate, polyvinylpyrrolidone, gum tragacanth and gum acacia; dispersing or wetting agents which may be a naturally occurring phosphatide such as lecithin, a condensation product of an alkylene oxide with a fatty acid, for example, polyoxyethylene stearate, a condensation product of ethylene oxide with a long chain aliphatic alcohol, for example, heptadecaethyleneoxycetanol, a condensation product of ethylene oxide with a partial ester derived form a fatty acid and a hexitol such as polyoxyethylene sorbitol monooleate, or a condensation product of an ethylene oxide with a partial ester derived from a fatty acid and a hexitol anhydride, for example polyoxyethylene sorbitan monooleate.

The sterile injectable preparation may also be a sterile injectable solution or suspension in a non-toxic parenterally acceptable diluent or solvent. Diluents and solvents that may be employed are, for example, water, Ringer's solution, and isotonic sodium chloride solution. In addition, sterile fixed oils are conventionally employed as solvents or suspending media. For this purpose, any bland, fixed oil may be employed including synthetic mono or diglycerides. In addition, fatty acids such as oleic acid may be used in the preparation of injectables.

A composition of the invention may also be administered in the form of suppositories for rectal administration of the drug. These compositions may be prepared by mixing the drug with a suitable non-irritating excipient which is solid at ordinary temperatures but liquid at the rectal temperature and will therefore melt in the rectum to release the drug. Such materials are, for example, cocoa butter and polyethylene glycol.

Another formulation employed in the methods of the present invention employs transdermal delivery devices (“patches”). Such transdermal patches may be used to provide continuous or discontinuous infusion of the peptides of the present invention in controlled amounts. The construction and use of transdermal patches for the delivery of pharmaceutical agents is well known in the art (see, e.g., U.S. Pat. No. 5,023,252, incorporated herein by reference). Such patches may be constructed for continuous, pulsatile, or on demand delivery of pharmaceutical agents.

Another formulation employs the use of biodegradable microspheres that allow controlled, sustained release of the peptides and PEGylated peptides of this invention. Such formulations can be comprised of synthetic polymers or copolymers. Such formulations allow for injection, inhalation, nasal or oral administration. The construction and use of biodegradable microspheres for the delivery of pharmaceutical agents is well known in the art (e.g., U.S. Pat. No. 6,706,289, incorporated herein by reference).

It may be desirable or necessary to introduce the pharmaceutical composition to the patient via a mechanical delivery device. The construction and use of mechanical delivery devices for the delivery of pharmaceutical agents is well known in the art. For example, direct techniques for administering a drug directly to the brain usually involve placement of a drug delivery catheter into the patient's ventricular system to bypass the blood-brain barrier. One such implantable delivery system, used for the transport of agents to specific anatomical regions of the body, is described in U.S. Pat. No. 5,011,472, incorporated herein by reference.

The compositions of the invention may also contain other conventional pharmaceutically acceptable compounding ingredients, generally referred to as carriers or diluents, as necessary or desired. Any of the compositions of this invention may be preserved by the addition of an antioxidant such as ascorbic acid or by other suitable preservatives. Conventional procedures for preparing such compositions in appropriate dosage forms can be utilized.

Commonly used pharmaceutical ingredients which may be used as appropriate to formulate the composition for its intended route of administration include: acidifying agents, for example, but are not limited to, acetic acid, citric acid, fumaric acid, hydrochloric acid, nitric acid; and alkalinizing agents such as, but are not limited to, ammonia solution, ammonium carbonate, diethanolamine, monoethanolamine, potassium hydroxide, sodium borate, sodium carbonate, sodium hydroxide, triethanolamine, trolamine.

The peptides identified by the methods described herein may be administered as the sole pharmaceutical agent or in combination with one or more other pharmaceutical agents where the combination causes no unacceptable adverse effects. For example, the peptides of this invention can be combined with known anti-diabetic, or with known anti-obesity, cardiovascular or other indication agents, and the like, as well as with admixtures and combinations thereof.

The peptides identified by the methods described herein may also be utilized, in free base form or in compositions, in research and diagnostics, or as analytical reference standards, and the like. Therefore, the present invention includes compositions which are comprised of an inert carrier and an effective amount of a peptide of the present invention. An inert carrier is any material which does not interact with the peptide to be carried and which lends support, means of conveyance, bulk, traceable material, and the like to the peptide to be carried. An effective amount of peptide is that amount which produces a result or exerts an influence on the particular procedure being performed.

Formulations suitable for subcutaneous, intravenous, intramuscular, and the like; suitable pharmaceutical carriers; and techniques for formulation and administration may be prepared by any of the methods well known in the art (see, e.g., Remington's Pharmaceutical Sciences, Mack Publishing Co., Easton, Pa., 20^{th} edition, 2000)

Peptides are known to undergo hydrolysis, deamidation, oxidation, racemization and isomerization in aqueous and non-aqueous environment. Degradation such as hydrolysis, deamidation or oxidation can readily detected by capillary electrophoresis. Enzymatic degradation notwithstanding, peptides having a prolonged plasma half-life, or biological resident time, should, at minimum, be stable in aqueous solution. For example, a peptide exhibits less than 10% degradation over a period of one day at body temperature or less than 5% degradation over a period of one day at body temperature. Stability (i.e., less than a few percent of degradation) over a period of weeks at body temperature will allow less frequent dosing. Stability in the magnitude of years at refrigeration temperature will allow the manufacturer to present a liquid formulation, thus avoid the inconvenience of reconstitution. Additionally, stability in organic solvent would provide peptide be formulated into novel dosage forms such as implant.

The structures, materials, compositions, and methods described herein are intended to be representative examples of the invention, and it will be understood that the scope of the invention is not limited by the scope of the examples. Those skilled in the art will recognize that the invention may be practiced with variations on the disclosed structures, materials, compositions and methods, and such variations are regarded as within the ambit of the invention.

The following examples are presented to illustrate the invention described herein, but should not be construed as limiting the scope of the invention in any way.

### EXAMPLES

In order that this invention may be better understood, the following examples are set forth. These examples are for the purpose of illustration only, and are not to be construed as limiting the scope of the invention in any manner. All publications mentioned herein are incorporated by reference in their entirety.

### Example 1

### Preparation of N-Terminal Modifying Compounds

Air and moisture sensitive liquids and solutions were transferred via syringe or cannula, and introduced into reaction vessels through rubber septa. Commercial grade reagents and solvents were used without further purification. The term “concentration under reduced pressure” refers to use of a Buchi rotary evaporator at approximately 15 mm of Hg. All temperatures are reported uncorrected in degrees Celsius (° C.). Thin layer chromatography (TLC) was performed on EM Science pre-coated glass-backed silica gel 60 A F-254 250 μm plates. Column chromatography (flash chromatography) was performed on a Biotage system using 32-63 micron, 60 A, silica gel pre-packed cartridges. Purification using preparative reversed-phase HPLC chromatography were accomplished using a Gilson 215 system and a YMC Pro-C18 AS-342 (150×20 mm I.D.) column. Typically, the mobile phase used was a mixture of H₂O (A) and MeCN (B). The water could be mixed or not with 0.1% TFA. A typical gradient was:

| Time | | | Flow |
|---|---|---|---|
| [min.] | A: % | B: % | [mL/min.] |
| 0.50 | 90.0 | 10.0 | 1.0 |
| 11.00 | 0.0 | 100.0 | 1.0 |
| 14.00 | 0.0 | 100.0 | 1.0 |
| 15.02 | 100.0 | 0.0 | 1.0 |

Electron impact mass spectra (EI-MS or GC-MS) were obtained with a Hewlett Packard 5989A mass spectrometer equipped with a Hewlett Packard 5890 Gas Chromatograph with a J & W DB-5 column (0.25 uM coating; 30 m×0.25 mm). The ion source was maintained at 250° C. and spectra were scanned from 50-800 amu at 2 sec per scan. High pressure liquid chromatography-electrospray mass spectra (LC-MS) were obtained using a Hewlett-Packard 1100 HPLC equipped with a quaternary pump, a variable wavelength detector set at 254 nm, a YMC pro C-18 column (2×23 mm, 120A), and a Finnigan LCQ ion trap mass spectrometer with electrospray ionization. Spectra were scanned from 120-1200 amu using a variable ion time according to the number of ions in the source. The eluents were A: 2% acetonitrile in water with 0.02% TFA and B: 2% water in acetonitrile with 0.018% TFA. Gradient elution from 10% to 95% B over 3.5 minutes at a flowrate of 1.0 mL/min was used with an initial hold of 0.5 minutes and a final hold at 95% B of 0.5 minutes. Total run time was 6.5 minutes. For consistency in characterization data, the retention time (RT) is reported in minutes at the apex of the peak as detected by the UV-Vis detector set at 254 nm.

Routine one-dimensional NMR spectroscopy was performed on 300 or 400 MHz Varian Mercury-plus spectrometers. The samples were dissolved in deuterated solvents obtained from Cambridge Isotope Labs, and transferred to 5 mm ID Wilmad NMR tubes. The spectra were acquired at 293 K. The chemical shifts were recorded on the ppm scale and were referenced to the appropriate residual solvent signals, such as 2.49 ppm for DMSO-_{d6}, 1.93 ppm for CD₃CN, 3.30 ppm for CD₃OD, 5.32 ppm for CD₂Cl₂, and 7.26 ppm for CDCl₃ for ¹H spectra, and 39.5 ppm for DMSO-_{d6}, 1.3 ppm for CD₃CN, 49.0 ppm for CD₃OD, 53.8 ppm for CD₂Cl₂, and 77.0 ppm for CDCl₃ for ¹³C spectra. General methods of preparation are illustrated in the reaction schemes, and by the specific preparative examples that follow.

### ABBREVIATIONS AND ACRONYMS

When the following abbreviations are used throughout the disclosure, they have the following meaning:
Ac acetyl
AcOH acetic acid
Boo t-butoxycarbonyl
Bu butyl
CDCl₃ deuterochloroform
Celite® registered trademark of Celite Corp. brand of diatomaceous earth
Cl chemical ionization
d doublet
dd doublet of doublet
ddd doublet of doublet of doublet
DME dimethoxyethane
DMF N,N-dimethyl formamide
DMSO dimethylsulfoxide
DMSO-d₆ dimethylsulfoxide-d₆
dppf 1,1′-bis(diphenylphosphino)ferrocene
EI electron impact ionization
EI-MS electron impact-mass spectrometry
Et ethyl
EtOH ethanol
EtOAc ethyl acetate
g gram
GC-MS gas chromatography-mass spectrometry
h hour(s)
¹H NMR proton nuclear magnetic resonance
Hex hexanes
HPLC high performance liquid chromatography
LC-MS liquid chromatography/mass spectroscopy
LDA lithium diisopropylamide
m multiplet
M molar
m/z mass over charge
Me methyl
MeCN acetonitrile
mg milligram
MHz megahertz
min minute(s)
mol mole
mmol millimole
MS mass spectrometry
N normal
NMR nuclear magnetic resonance
NaOAc sodium acetate
Pd/C palladium on carbon
PdCl₂(dppf).CH₂Cl₂ [1,1′-bis(diphenylphosphino)ferrocene]dichloropalladium (II) complex with dichloromethane (1:1)
Ph phenyl
PPh₃ triphenylphosphine
ppm parts per million
Pr propyl
q quartet
qt quintet
quant. quantitative
R_{I} TLC retention factor
rt room temperature
RT retention time (HPLC)
s singlet
TFA trifluoroacetic acid
THF tetrahydrofuran
TLC thin layer chromatography
TMS tetramethylsilane
v/v volume per unit volume
vol volume
w/w weight per unit weight

### Example 1A

### Preparation of (2-Mercapto-1H-benzimidazol-1-yl)acetic acid

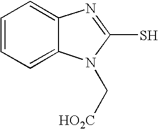

### Step 1. Preparation of Methyl (2-chloro-1H-benzimidazol-1-yl)acetate

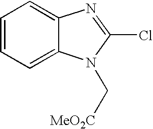

NaH (157.3 mg of a 60% dispersion in mineral oil, 3.93 mmol) was suspended in dry DMF (5 mL) and hexanes (0.5 mL). 2-chlorobenzimidazole (500 mg, 3.28 mmol) was added, and the resulting solution was allowed to stir at rt for 1 h. Methyl bromoacetate (0.37 mL, 3.93 mmol) was added, and the solution was allowed to stir overnight at rt. The reaction mixture was then diluted with water, and the resulting tan precipitate was collected by filtration, triturated with ether, and dried, yielding 385 mg (52%) of crude material. ¹H NMR (400 MHz, CD₃CN) [symbol] 3.78 (s, 3H), 5.03 (s, 2H), 7.28-7.38 (m, 2H), 7.43 (d, 1H), 7.65 (d, 1H).

### Step 2. Preparation of Methyl (2-mercapto-1H-benzimidazol-1-yl)acetate

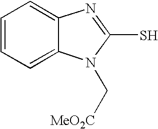

Methyl (2-chloro-1H-benzimidazol-1-yl)acetate (150 mg, 0.67 mmol) and thiourea (101.7 mg, 1.34 mmol) were heated to reflux in ethanol (30 mL) overnight. The reaction mixture was concentrated, and the residue was triturated with water and dried, yielding 142.5 mg (96%) of crude product. ¹H NMR (400 MHz, CD₃CN) [symbol] 3.78 (s, 3H), 5.03 (s, 2H), 7.20-7.35 (m, 4H), 10.43 (bs, 1H).

### Step 3. Preparation of (2-Mercapto-1H-benzimidazol-1-yl)acetic acid

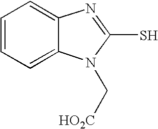

Methyl (2-mercapto-1H-benzimidazol-1-yl)acetate (143 mg, 0.64 mmol) was dissolved in THF (1 mL), MeOH (1 mL), and water (0.5 mL). The solution was treated with LiOH (17.0 mg, 0.71 mmol) and heated to 80° C. for 4 h. The pH was then adjusted to pH 4 with 1N HCl. The solution was diluted with water and extracted with 5% EtOH/EtOAc. The organics were dried over MgSO₄ and concentrated in vacuo, yielding 75.0 mg (56%) of the desired product. LC/MS m/z 209.1 (M+H)⁺; RT 1.08 min. ¹H NMR (400 MHz, CD₃CN) [symbol] 5.03 (s, 2H), 7.20-7.35 (m, 4H), 10.43 (bs, 1H).

### Example 1B

### Preparation of Lithium 2-{[2-(tritylthio)ethyl]amino}nicotinate

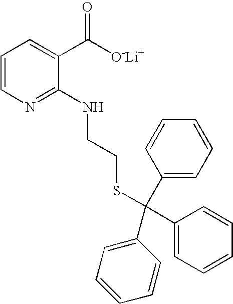

### Step 1. Preparation of Ethyl 2-{[2-(tritylthio)ethyl]amino}nicotinate

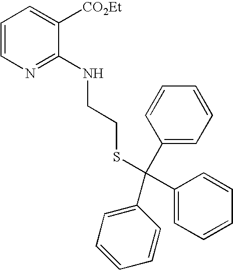

Ethyl 2-chloronicotinate (100 mg, 0.54 mmol) was combined with 2-(tritylthio)ethanamine (344 mg, 1.08 mmol), cesium carbonate (438 mg, 1.35 mmol), and [1,1′-bis(diphenylphosphino) ferrocene]dichloropalladium(II) complex with dichloromethane (1:1) (110 mg, 0.13 mmol). The solids were dissolved in dioxane (2 mL), water (1 mL), and heated to reflux over 48 h. The reaction mixture was then diluted with EtOAc and washed with water and brine. The organics were dried over Na₂SO₄ and concentrated in vacuo. The crude residue was purified by Biotage column chromatography (10% EtOAc/hexane), yielding 215 mg (85%) of the desired product. ¹H NMR (400 MHz, CD₂Cl₂) [symbol] 1.40 (t, 3H), 2.45 (t, 2H), 3.38 (t, 2H), 4.37 (q, 2H), 7.03 (m, 1H), 7.18-7.45 (m, 16H), 8.18 (d, 1H), 8.45 (d, 1H).

### Step 2. Preparation of Lithium 2-{[2-(tritylthio)ethyl]amino}nicotinate

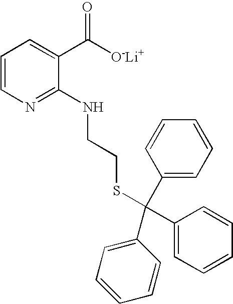

Ethyl 2-{[2-(tritylthio)ethyl]amino}nicotinate (215 mg, 0.46 mmol) was dissolved in THF (1 mL), MeOH (1 mL), and water (0.5 mL). The solution was treated with LiOH (12.1 mg, 0.50 mmol) and heated to 80° C. for 4 h. The crude aqueous mixture was extracted with ether to remove impurities. The solution was then diluted with water and extracted with 5% EtOH/EtOAc. The EtOH/EtOAc extracts were dried over MgSO₄ and concentrated in vacuo, yielding 55.0 mg (27%) of the desired product. LC/MS m/z 440.7 (M+H)⁺; RT 2.17 min. ¹H NMR (400 MHz, CD₃CN) [symbol] 2.38 (t, 2H), 3.32 (t, 2H), 7.08 (m, 1H), 7.18-7.45 (m, 16H), 8.19 (d, 1H), 8.45 (d, 1H).

### Example 1C

### Preparation of Lithium 1-[2-(tritylthio)ethyl]-1H-imidazole-2-carboxylate

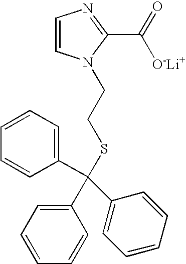

### Step 1. Preparation of 1,1′,1″-{[(2-bromoethyl)thio]methanetriyl}tribenzene

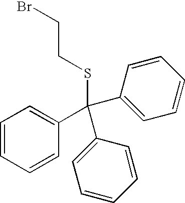

Triphenylmethylmercaptan (3.00 g, 10.9 mmol) was dissolved in THF (10 mL) and cooled to 0° C. Lithium hexamethyldisilazide (10.85 ml of a 1M solution in THF) was added, and the reaction mixture was allowed to stir for 30 min. The cooling bath was removed and dibromoethane (1.12 mL, 13.0 mmol) was added. The reaction mixture was allowed to stir at rt for an additional 30 min and was concentrated in vacuo. The crude residue was dissolved in ethyl acetate and washed with water and brine. The organics were dried over Na₂SO₄ and concentrated, yielding 3.44 g crude material (80% pure by ¹H NMR integration). This material was used without further purification. ¹H NMR (400 MHz, CD₂Cl₂) [symbol] 2.75 (t, 2H), 2.90 (t, 2H), 7.20-7.45 (m, 18.6H).

### Step 2. Preparation of 1-[2-(Tritylthio)ethyl]-1H-imidazole

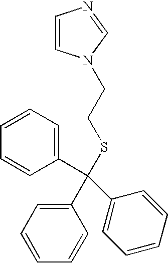

NaH (70.5 mg of a 60% suspension in mineral oil, 1.76 mmol) was suspended in dry DMF (3 mL) and hexanes (0.5 mL). Imidazole (100 mg, 1.47 mmol) was added, and the resulting solution was allowed to stir at rt for 1 h. 1,1′,1″-{[(2-bromoethyl)thio]methanetriyl}tribenzene (845 mg, 1.76 mmol) was added, and the solution was allowed to stir at rt overnight. The reaction mixture was diluted with water and extracted with EtOAc. The organics were washed with water and brine and dried over Na₂SO₄. The crude product was purified by Biotage column chromatography (50% EtOAc/hexane, 1% Et₃N), yielding 280 mg (51%) of the desired product ¹H NMR (400 MHz, CD₂Cl₂) [symbol] 2.63 (t, 2H), 3.48 (t, 2H), 6.70 (s, 1H), 6.92 (s, 1H), 7.19 (s, 1H), 7.22-7.45 (m, 15H).

### Step 3. Preparation of Lithium 1-[2-(tritylthio)ethyl]-1H-imidazole-2-carboxylate

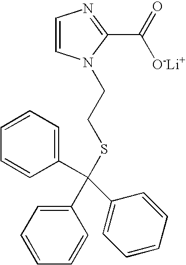

1-[2-(Tritylthio)ethyl]-1H-imidazole (140 mg, 0.38 mmol) was dissolved in dry dichloromethane (1.5 mL) and treated with trichloroacetyl chloride (0.06 mL, 0.57 mmol) and N,N-diisopropylethylamine (0.07 mL, 0.42 mmol). The reaction mixture was allowed to stir overnight at rt. The reaction mixture was then concentrated in vacuo. The crude residue was dissolved in THF (1 mL), MeOH (1 mL), and water (0.5 mL), treated with LiOH (18.1 mg, 0.76 mmol) and allowed to stir at 80° C. for 4 h. The crude reaction mixture was then concentrated in vacuo. Purification by HPLC gave 70 mg (44%) of the desired product. LC/MS m/z 414.9 (M+H)⁺; RT 2.76 min. ¹H NMR (400 MHz, CD₃OD) [symbol] 2.68 (t, 2H), 4.18 (t, 2H), 6.72 (s, 1H), 6.85 (s, 1H), 7.19-7.40 (m, 15H).

### Example 1D

### Preparation of Lithium 4-{[2-(tritylthio)ethyl]amino}pyrimidine-5-carboxylate

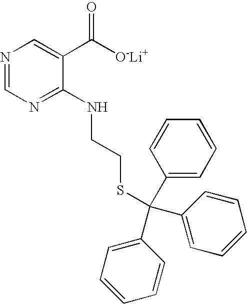

### Step 1. Preparation of Ethyl 4-hydroxypyrimidine-5-carboxylate

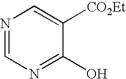

Diethyl malonate (3.14 mL, 20.7 mmol) was combined with N,N′,N″-methylidynetrisformamide (3.00 g, 20.7 mmol), and p-toluenesulfonic acid (356 mg, 2.07 mmol), and the reaction mixture was heated to 18° C. for 4 h. The resulting red oil was allowed to cool to rt overnight. The crude reaction mixture was dissolved in a minimum amount of water and allowed to crystallize overnight. The solids were collected by filtration, washed with water, and dried, yielding 822 mg (24%) of the desired product. ¹H NMR (400 MHz, CD₃OD) [symbol] 1.38 (t, 3H), 4.32 (q, 2H), 8.32 (s, 1H), 8.60 (s, 1H).

### Step 2. Preparation of ethyl 4-chloropyrimidine-5-carboxylate

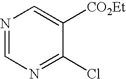

Ethyl 4-hydroxypyrimidine-5-carboxylate (380 mg, 2.26 mmol) was dissolved in THF (5 mL) and treated with thionyl chloride (1.65 ml, 22.6 mmol). The solution was heated to reflux for 4 h and then concentrated in vacuo, yielding 417 mg (99%) of the crude product. This material was used without purification or characterization.

### Step 3. Preparation of ethyl 4-{[2-(tritylthio)ethyl]amino}pyrimidine-5-carboxylate

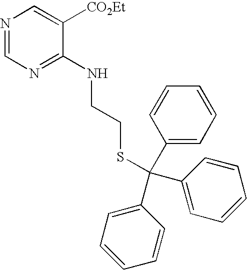

Ethyl 4-chloropyrimidine-5-carboxylate (200 mg, 1.07 mmol) was dissolved in THF (2 mL), and 2-(tritylthio)ethanamine (514 mg, 1.61 mmol) and N,N-diisopropylethylamine (0.56 ml, 3.22 mmol) were added. The reaction mixture was heated to reflux overnight and then concentrated in vacuo. The crude residue was purified by Biotage column chromatography, yielding 230 mg (46%) product. Product confirmed by HNMR. ¹H NMR (400 MHz, CD₂Cl₂) [symbol] 1.42 (t, 3H), 2.48 (bs, 2H), 3.40 (bs, 2H), 4.40 (q, 2H), 7.18-7.50 (m, 16H), 8.85 (s, 1H), 8.96 (s, 1H).

### Step 4. Preparation of Lithium 4-{[2-(tritylthio)ethyl]amino}pyrimidine-5-carboxylate

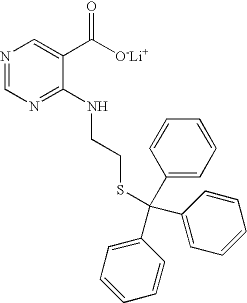

Ethyl 4-{[2-(tritylthio)ethyl]amino}pyrimidine-5-carboxylate (230 mg, 0.49 mmol) was dissolved in THF (1 mL), MeOH (1 mL), and water (0.5 mL). The solution was treated with LiOH (23.5 mg, 0.98 mmol) and allowed to stir at rt for two days. The crude reaction mixture was then diluted with water and extracted with EtOAc. The organic extracts were dried over Na₂SO₄ and concentrated in vacuo, yielding 180 mg (82%) of the desired product. LC/MS m/z 441.6 (M+H)⁺; RT 2.54 min. ¹H NMR (400 MHz, CD₃OD) [symbol] 2.43 (t, 2H), 3.32 (m beneath solvent peak), 7.10-7.42 (m, 1H), 8.70 (d, 2H).

### Example 1E

### Preparation of Lithium 1-[2-(tritylthio)ethyl]-1H-benzimidazole-2-carboxylate

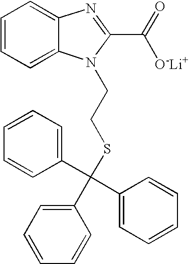

### Step 1. Preparation of ethyl 1H-benzimidazole-2-carboxylate

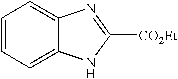

1H-Benzimidazole-2-carboxylic acid (500 mg, 3.08 mmol) was suspended in EtOH (5 mL), treated with thionyl chloride (1.12 mL, 15.4 mmol), and heated to reflux overnight. The reaction mixture was concentrated in vacuo, yielding 644 mg (99%) of the crude product. ¹H NMR (400 MHz, CD₂Cl₂) [symbol] 1.32 (t, 3H), 4.38 (q, 2H), 7.30 (m, 2H), 7.63 (m, 2H).

### Step 2. Preparation of ethyl 1-(2-bromoethyl)-1H-benzimidazole-2-carboxylate

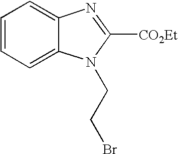

NaH (504 mg of a 60% suspension in mineral oil, 1.07 mmol) was suspended in dry DMF (1.5 mL). Ethyl 1H-benzimidazole-2-carboxylate (170 mg, 0.89 mmol) was added, and the solution was allowed to stir at rt for 1 h. 1,2-dibromoethane (0.23 mL, 2.7 mmol) was added, and the solution was heated to 50° C. overnight. The reaction mixture was diluted with water and extracted with EtOAc. The organic extracts were dried over Na₂SO₄, concentrated in vacuo, and purified by Biotage column chromatography (15% EtOAc/hexanes), yielding 100 mg (38%) of the desired product. ¹H NMR (400 MHz, CD₂Cl₂) [symbol] 1.48 (t, 3H), 3.80 (t, 2H), 4.50 (q, 2H), 5.02 (t, 2H), 7.39 (t, 1H), 7.45 (t, 1H), 7.53 (d, 1H), 7.87 (d, 1H).

### Step 3. Preparation of ethyl 1-[2-(tritylthio)ethyl]-1H-benzimidazole-2-carboxylate

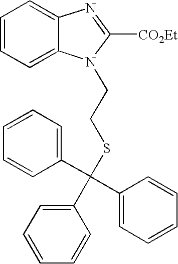

A solution of ethyl 1-(2-bromoethyl)-1H-benzimidazole-2-carboxylate (100 mg, 0.34 mmol), triphenylmethylmercaptan (112 mg, 0.40 mmol), and N,N-diisopropylethylamine (0.07 ml, 0.40 mmol) in THF (1 mL) were allowed to stir for 2 h at rt. In a separate flask, a solution of triphenylmethylmercaptan (112 mg, 0.40 mmol) in THF (1 mL) was treated with lithium hexamethyldisilazide (0.40 mL of a 1M solution in THF) and allowed to stir for 10 min at rt. This solution was added to the original reaction mixture, immediately resulting in a red solution that was allowed to stir at rt overnight. The reaction mixture was concentrated in vacuo, and the crude residue was purified by Biotage column chromatography, yielding 80 mg (48%) of the desired product. ¹H NMR (400 MHz, CD₂Cl₂) [symbol] 1.44 (t, 3H), 2.78 (t, 2H), 4.40-4.52 (m, 4H), 7.03 (m, 1H), 7.20-7.40 (m, 15H), 7.42 (m, 1H), 7.82 (m, 1H).

### Step 4. Preparation of Lithium 1-[2-(tritylthio)ethyl]-1H-benzimidazole-2-carboxylate

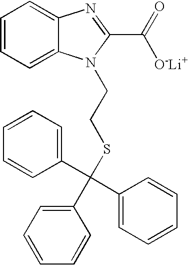

Ethyl 1-[2-(tritylthio)ethyl]-1H-benzimidazole-2-carboxylate (75 mg, 0.15 mmol) was dissolved in THF (1 mL), MeOH (1 mL), and water (0.5 mL). The solution was treated with LiOH (7.3 mg, 0.30 mmol) and allowed to stir at rt for 2 days. The crude reaction mixture was then diluted with water and extracted with EtOAc. The organic extracts were dried over Na₂SO₄ and concentrated in vacuo, yielding 74 mg (99%) of the desired product. LC/MS m/z 464.9 (M+H)⁺; RT 3.16 min. ¹H NMR (400 MHz, CD₃OD) [symbol] 2.72 (t, 2H), 4.58 (t, 2H), 6.98 (m, 1H), 7.15-7.30 (m, 16H), 7.38 (d, 1H), 7.63 (m, 1H).

### Example 1F

### Preparation of (2-Mercapto-1H-imidazol-1-yl)acetic Acid

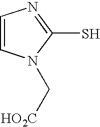

### Step 1. Preparation of ethyl N-(2,2-dimethoxyethyl)glycinate hydrochloride

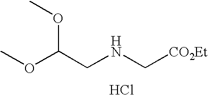

Bromoacetaldehyde dimethylacetal (0.85 mL, 7.2 mmol) was added to a solution of glycine ethyl ester hydrochloride (500 mg, 3.58 mmol) and N,N-diisopropylethylamine (1.37 mL, 7.88 mmol) in THF (4 mL) and EtOH (1 mL). The reaction mixture was heated to 70° C. and allowed to stir overnight. The solution was diluted with water and extracted with dichloromethane. The organic extracts were dried over Na₂SO₄ and concentrated in vacuo. The crude residue was treated with 1N HCl in ether, and the resulting precipitate was collected by filtration and dried, yielding 371 mg (23%) of the desired product. ¹H NMR (400 MHz, CD₃CN) [symbol] 1.30 (t, 3H), 3.18 (bs, 2H), 3.42 (s, 6H), 3.85 (bs, 2H), 4.27 (q, 2H), 4.92 (t, 1H), 9.40 (bs, 2H).

### Step 2. Preparation of ethyl (2-mercapto-1H-imidazol-1-yl)acetate

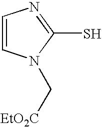

Ethyl N-(2,2-dimethoxyethyl)glycinate hydrochloride (370 mg, 1.63 mmol) was dissolved in EtOH (2 mL) and treated with a solution of potassium thiocyanate (237 mg, 2.44 mmol) in EtOH (8 mL). The pink suspension was heated to reflux overnight. Concentrated HCl (0.136 mL, 1.63 mmol) was added, and the solution was allowed to reflux for 3 h. The reaction mixture was concentrated in vacuo, and the resulting solid was recrystallized from EtOAc, yielding 130 mg (43%) of the desired product. ¹H NMR (400 MHz, CD₃CN) [symbol] 1.33 (t, 3H), 4.20 (q, 2H), 4.77 (s, 2H), 6.77 (s, 1H), 6.83 (s, 1H), 9.92 (bs, 1H).

### Step 3. Preparation of (2-Mercapto-1H-imidazol-1-yl)acetic acid

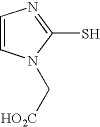

Ethyl (2-mercapto-1H-imidazol-1-yl)acetate (130 mg, 0.70 mmol) was dissolved in THF (1 mL), MeOH (1 mL), and water (0.5 mL). The solution was treated with LiOH (33.4 mg, 1.40 mmol) and allowed to stir at rt for 2 days. The crude reaction mixture was acidified to pH 2 with 2N HCl, diluted with water, and extracted with 5:1 EtOAc/EtOH. The organic extracts were dried over Na₂SO₄ and concentrated in vacuo, yielding 70 mg (63%) of the desired product. LC/MS m/z 159.1 (M+H)⁺; RT 1.05 min. ¹H NMR (400 MHz, CD₃OD) [symbol] 4.82 (s, 2H), 6.82 (s, 1H), 6.99 (s, 1H).

### Example 1G

### Preparation of Lithium ({1-[2-(tritylthio)ethyl]-1H-imidazol-2-yl}thio)acetate

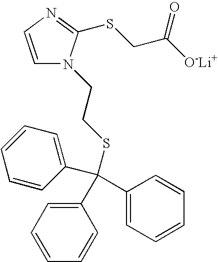

### Step 1. Preparation of methyl (1H-imidazol-2-ylthio)acetate

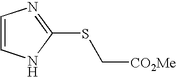

2-Thioimidazole (300 mg, 3.00 mmol) was dissolved in THF (3 mL), treated with N,N-diisopropylethylamine (0.63 mL, 3.59 mmol) and methyl bromoacetate (0.31 mL, 3.3 mmol), and allowed to stir at rt for 1 h. The solid precipitate was filtered off, and the filtrate was diluted with EtOAc. The organics were washed with water, dried over Na₂SO₄ and concentrated in vacuo, yielding 385 mg (75%) of the desired product. ¹H NMR (400 MHz, CD₃CN) [symbol] 3.68 (s, 3H), 3.82 (s, 2H), 7.05 (s, 2H).

### Step 2. Preparation of Methyl ({1-[2-(tritylthio)ethyl]-1H-imidazol-2-yl}thio)acetate

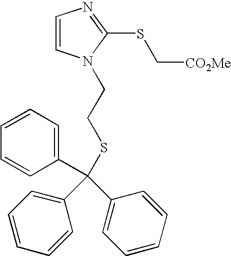

NaH (55.7 mg of a 60% suspension in mineral oil, 1.39 mmol) was suspended in dry DMF (1.5 mL). Methyl (1H-imidazol-2-ylthio)acetate (200 mg, 1.16 mmol) was added, and the resulting solution was allowed to stir at rt for 1 h. 1,1′,1″-{[(2-bromoethyl)thio]methanetriyl}tribenzene (668 mg, 1.39 mmol) was added, and the reaction mixture was heated to 50° C. overnight. The reaction mixture was diluted with water and extracted with EtOAc. The organic extracts were washed with water and brine, dried over MgSO₄, and concentrated in vacuo. The crude residue was purified by Biotage column chromatography (20% EtOAc/hexanes), yielding 180 mg (33%) of the desired product. ¹H NMR (400 MHz, CD₂Cl₂) [symbol] 2.60 (t, 2H), 3.65 (m, 5H), 3.78 (s, 2H), 6.69 (s, 1H), 6.96 (s, 1H), 7.20-7.40 (m, 15H).

### Step 3. Preparation of Lithium ({1-[2-(tritylthio)ethyl]-1H-imidazol-2-yl}thio)acetate

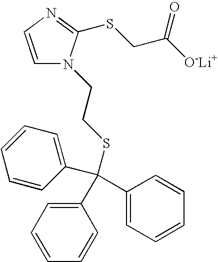

Methyl ({1-[2-(tritylthio)ethyl]-1H-imidazol-2-yl}thio)acetate (180 mg, 0.38 mmol) was dissolved in THF (1 mL), MeOH (1 mL), and water (0.5 mL). The solution was treated with LiOH (18.2 mg, 0.76 mmol) and allowed to stir at rt overnight. The reaction mixture was diluted with water and extracted with EtOAc. The organic extracts were dried over Na₂SO₄ and concentrated in vacuo. Purification of the crude material by HPLC gave 61 mg (34%) of the desired product. LC/MS m/z 460.9 (M+H)⁺; RT 2.81 min. ¹H NMR (400 MHz, CD₃OD) [symbol] 2.59 (t, 2H), 3.60 (s, 2H), 3.92 (t, 2H), 6.89 (s, 1H), 6.92 (s, 1H), 7.19-7.35 (m, 15H).

### Example 1H

### Preparation of Lithium 3-(2-tritylsulfanylethylamino)pyrazine-2-carboxylate

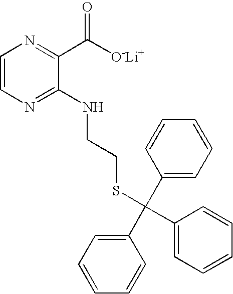

### Step 1. Preparation of 2-tritylsulfanylethylamine

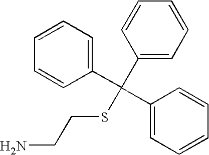

A suspension of cysteamine hydrochloride (1.14 g, 9.80 mmol) and triethylamine (3.0 mL, 21.6 mmol) in dichloromethane (20 mL) was stirred at rt for 10 min. N,O-Bis(trimethylsilyl) acetamide (1.99 g, 9.80 mmol) was then added, and the reaction was allowed to stir for 30 min under nitrogen. The reaction mixture was cooled to 0° C. and trityl chloride (2.46 g, 8.82 mmol) was added in one portion. The suspension was allowed to warm to rt and stir for 16 h. The reaction mixture was quenched with water (15 mL). The mixture was washed successively with 1N HCl (2×10 mL), water (2×10 mL), 15% ammonia solution (4 mL), and water (5×20 mL). The organics were dried over Na₂SO₄ and concentrated in vacuo to give 1.9 g (61%) of a light brown oil. ¹H NMR (400 MHz, CDCl₃) δ7.70-7.18 (m, 15H), 2.71 (t, 2H), 2.4 (b, 2H).

### Step 2. Preparation of 3-bromopyrazine-2-carboxylic acid methyl ester

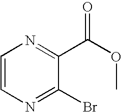

Bromine (3.91 g, 24.46 mmol) was added dropwise to a stirred mixture of 3-aminopyrazine-2-carboxylic acid methyl ester (1.27 g, 8.29 mmol) and hydrobromic acid (4.70 mL, 41.5 mmol) at 0° C. A solution of sodium nitrite (1.44 g, 20.9 mmol) in water (6 mL) was then added dropwise. The reaction mixture was stirred for 15 min, brought to pH 8 with NaHCO₃ (saturated, aqueous), and extracted with ethyl acetate (80 mL) and chloroform (50 mL). The combined organics were dried over MgSO₄ and concentrated in vacuo to give 1.13 g (63%) of an orange oil which solidified on standing. LC-MS m/z 217 (M+H⁺); RT 1.15 min.

### Step 3. Preparation of 3-(2-tritylsulfanylethylamino)pyrazine-2-carboxylic acid methyl ester

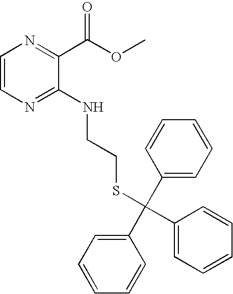

A solution of 3-bromopyrazine-2-carboxylic acid methyl ester (0.10 g, 0.45 mmol), 2-tritylsulfanylethylamine (0.29 g, 0.90 mmol) and triethylamine (0.06 mL, 0.44 mmol) in acetonitrile (5 mL) was heated to reflux for 18 h under argon. The mixture was concentrated under reduced pressure and purified by column chromatography (3:1 Hex:EtOAc), yielding 0.058 g (28%) of the desired product. ¹H NMR (400 MHz, CD₂Cl₂) δ 7.95 (s, 1H), 8.44 (s, 1H), 8.31 (s, 1H), 7.57.18 (m, 15H), 4.20 (s, 3H), 3.36 (t, 2H), 2.50 (t, 2H).

### Step 4. Preparation of lithium 3-(2-tritylsulfanylethylamino)pyrazine-2-carboxylate

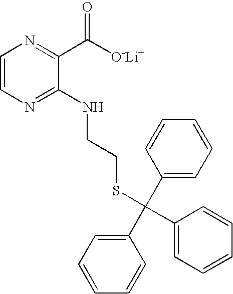

A mixture of 3-(2-tritylsulfanylethylamino)pyrazine-2-carboxylic acid methyl ester (0.12 g, 0.27 mmol) and LiOH (0.030 g, 1.3 mmol) in THF (5 mL), methanol (5 mL), and water (2.5 mL) was stirred at rt for 18 h. The reaction mixture was concentrated and purified by HPLC, yielding 0.075 g (63%) of the desired product. ¹H NMR (400 MHz, CD₃OD) δ 8.26 (s, 1H), 8.15 (s, 1H), 7.42-7 (m, 15H), 3.30 (t, 2H), 2.40 (t, 2H).

### Example 1I

### Preparation of 4-mercaptothiazole-5-carboxylic acid

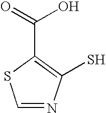

### Step 1. Preparation of 4-(2-methoxycarbonylethylsulfanyl)thiazole-5-carboxylic acid ethyl ester

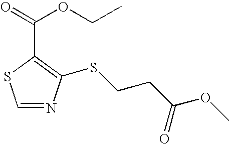

A solution of ethyl isocyanoacetate (0.92 g, 7.7 mmol) in THF (8 mL) was added dropwise to a suspension of potassium tert-butoxide (1.0 g, 8.5 mmol) in THF (6 mL) at −40° C. The mixture was cooled to −60° C., and a solution of carbon disulfide (0.59 g, 7.7 mmol) in THF (8 mL) was added dropwise while keeping the temperature below −50° C. The mixture was warmed to 10° C. and methyl 3-bromopropionate (1.33 g, 7.70 mmol) was added. The mixture was allowed to stir for 2 h and was concentrated in vacuo. The product was recrystallized from dichloromethane/hexanes to give 1.28 g (60%) of the desired product as a white solid. LC-MS m/z 276 (M+H⁺); RT 1.65 min.

### Step 2. Preparation of 4-mercaptothiazole-5-carboxylic acid ethyl ester

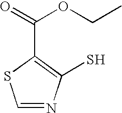

Sodium hydroxide (0.14 g, 3.5 mmol) was added to a solution of 4-(2-methoxycarbonylethylsulfanyl)thiazole-5-carboxylic acid ethyl ester (0.96 g, 3.5 mmol) in methanol (13.6 mL). The mixture was refluxed for 1 h and then concentrated in vacuo. The residue was taken up in ethyl acetate/water and the pH adjusted to 2 with 2 N HCl. The organic layer was isolated and concentrated in vacuo, yielding 0.66 g (100%) of the desired product, which was used without further purification. LC-MS m/z 189 (M+H⁺); RT 1.47 min.

### Step 3. Preparation of 4-mercaptothiazole-5-carboxylic acid

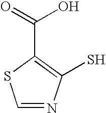

Sodium hydroxide (0.25 g, 6.3 mmol) was added to a solution of 4-mercaptothiazole-5-carboxylic acid ethyl ester (0.60 g, 3.2 mmol) in methanol (5 mL) and water (5 mL), and the mixture was heated to 80° C. for 3 h. Upon cooling to rt, the reaction mixture was concentrated in vacuo. The residue was acidified with 2 N HCl and extracted with dichloromethane. The organic layer was dried over MgSO₄, concentrated in vacuo, and purified by HPLC to give 0.059 g (12%) of the desired product. ¹H NMR (400 MHz, CD₃OD) δ 8.95 (s, 1H); LC-MS m/z 162 (M+H⁺); RT 1.01 min.

### Example 1J

### Preparation of 2-mercaptothiazole-5-carboxylic acid

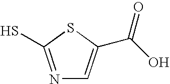

### Step 1. Preparation of 2-mercaptothiazole-5-carboxylic acid methyl ester

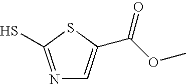

A mixture of 2-bromothiazole-5-carboxylic acid methyl ester (0.40 g, 1.8 mmol) and thiourea (0.16 g, 2.1 mmol) in ethanol (6 mL) was heated to reflux for 2 h. The mixture was allowed to cool to rt, and the resulting suspension was filtered to give 0.19 g (61%) of the desired product as a yellow solid. LC-MS m/z 176.1 (M+H⁺); RT 1.17 min.

### Step 2. Preparation of 2-mercaptothiazole-5-carboxylic acid

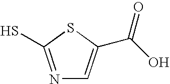

Sodium hydroxide (0.08 g, 1.9 mmol) was added to a solution of 2-mercaptothiazole-5 carboxylic acid methyl ester (0.17 g, 0.97 mmol) in methanol (5 mL) and water (5 mL). The reaction mixture was stirred at rt for 3 h and concentrated in vacuo. The residue was acidified with 2 N HCl and the resulting suspension was filtered to give 0.061 g (39%) of the desired product as an off-white solid. ¹H NMR (400 MHz, CD₃OD) δ 7.80 (s, 1H); LC-MS m/z 161 (M+H⁺); RT 1.02 min.

### Example 1K

### Preparation of Lithium 2-(2-tritylsulfanylethylamino)thiazole-5-carboxylate

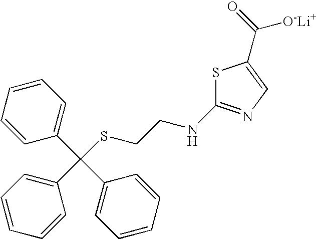

### Step 1. Preparation of 2-(2-tritylsulfanyl-ethylamino)-thiazole-5-carboxylic acid methyl ester

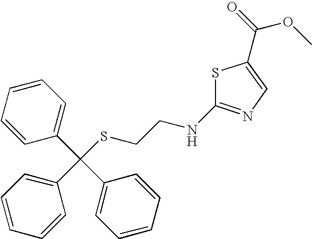

A solution of 2-bromothiazole-5-carboxylic acid methyl ester (0.20 g, 0.88 mmol), 2-tritylsulfanylethylamine (0.42 g, 1.3 mmol), and triethylamine (0.12 mL, 0.88 mmol) in acetonitrile (5 mL) was heated to reflux for 18 h under argon. The mixture was concentrated in vacuo and purified by column chromatography (3:1 Hex:EtOAc) to give 0.12 g (29%) of the desired product. ¹H NMR (400 MHz, CD₂Cl₂) δ 8.15 (s, 1H), 7.60-7.15 (m, 15H), 4.20 (s, 3H), 3.45 (t, 2H), 2.50 (br, 2H).

### Step 2. Preparation of lithium 2-(2-tritylsulfanylethylamino)-thiazole-5-carboxylate

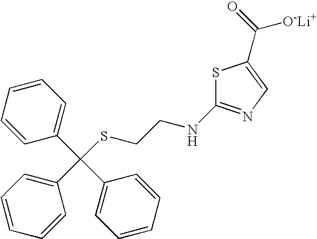

A mixture of 2-(2-tritylsulfanylethylamino)-thiazole-5-carboxylic acid methyl ester (0.12 g, 0.26 mmol) and LiOH (0.03 g, 1.3 mmol) in THF (5 mL), methanol (5 mL) and water (2.5 mL) was stirred at rt for 18 h. The reaction mixture was concentrated in vacuo and purified by HPLC to give 0.087 g (75%) of the desired product. ¹H NMR (400 MHz, CD₂Cl₂) δ 7.90 (s, 1H), 7.40-7.10 (m, 15H), 3.30 (s, 3H), 3.45 (t, 2H), 2.40 (br, 2H).

### Example 1L

### Preparation of 2-mercapto-6-methylpyrimidine-4-carboxylic acid

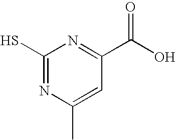

### Step 1. Preparation of 2-carbamimidoylsulfanyl-6-methylpyrimidine-4-carboxylic acid methyl ester hydrobromide

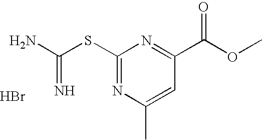

A mixture of 2-chloro-6-methylpyrimidine-4-carboxylic acid methyl ester (0.50 g, 2.7 mmol) and thiourea (0.41 g, 5.4 mmol) in ethanol (8 mL) was heated to reflux for 16 h and then concentrated in vacuo. Attempt to purify the product by recrystallization (methanol/ether) gave 0.229 g (38%) of an oil which was confirmed as the desired product. LC-MS m/z 226.9 (M+H⁺); RT 1.07 min.

### Step 2. Preparation of 2-mercapto-6-methylpyrimidine-4-carboxylic acid

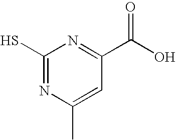

A mixture of 1 N sodium hydroxide (6.98 mL, 6.98 mmol) and 2-carbamimidoylsulfanyl-6-methylpyrimidine-4-carboxylic acid methyl ester hydrobromide (0.37 g, 1.4 mmol) was heated to reflux for 2 h. Upon cooling to rt, the reaction mixture was concentrated in vacuo. The residue was taken up in water and MP-TsOH (Argonaut technologies) was added. The mixture was stirred for 18 h until a pH of 4 was achieved. The mixture was filtered, and the filtrate was concentrated in vacuo. The residue was taken up in methanol, filtered, and the filtrate was concentrated in vacuo to give 0.139 g (59%) of the desired product as a brownish solid. ¹H NMR (400 MHz, CD₃OD) δ 7.10 (s, 1H), 3.40 (s, 3H); LC-MS m/z 171 (M+H⁺); RT 1.15 min.

### Example 1M

### Preparation of 5-mercaptonicotinic acid

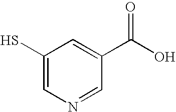

### Step 1. Preparation of 5-tert-butylsulfanylnicotinonitrile

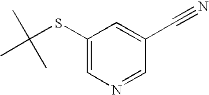

tert-Butylthiol (0.43 g, 4.8 mmol) was added to a suspension of NaH (0.19 g, 4.8 mmol) in DMF (15 mL), and the reaction mixture was heated at 50° C. for 1 h. 5-Bromonicotinonitrile (0.60 g, 3.2 mmol) was added to the resulting suspension, and the reaction mixture was heated at 120° C. for 5 h. Upon cooling to rt, the mixture was concentrated in vacuo and purified by HPLC, yielding 0.329 g (54%) of the desired product as an off-white solid. LC-MS m/z 193 (M+H⁺); RT 2.90 min.

### Step 2. Preparation of 5-tert-butylsulfanylnicotinic acid

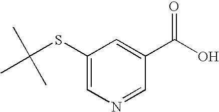

A mixture of 5-tert-butylsulfanylnicotinonitrile (0.43 g, 2.2 mmol) and sodium hydroxide (0.89 g, 22 mmol) in ethanol (5 mL) and water (5 mL) was heated to reflux for 1 h. Upon cooling to rt, the reaction mixture was diluted with water and was extracted with ether. The aqueous layer was acidified with 2 N HCl and was extracted with dichloromethane. The dichloromethane extracts were dried over MgSO₄ and concentrated to give 0.433 g (79%) of the desired product as a white solid. LC-MS m/z 212 (M+H⁺); RT 2.36 min.

### Step 3. Preparation 5-mercaptonicotinic acid

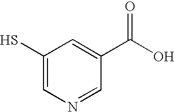

A solution of 5-tert-butylsulfanylnicotinic acid (0.30 g, 1.2 mmol) in 2 N HCl (9 mL, 18 mmol) was heated to reflux for 32 h. Upon cooling to rt, the reaction mixture was concentrated in vacuo to give 0.054 g (28%) of the desired product. ¹H NMR (400 MHz, CD₃OD) δ 9.00-8.80 (m, 2H), 8.40 (d, 1H); LC-MS m/z 156 (M+H⁺); RT 2.37 min.

### Example 1N

### Preparation of 5-isopropyl-2-mercaptothiazole-4-carboxylic acid

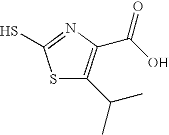

### Step 1. Preparation of 2-chloro-4-methyl-3-oxopentanoic acid ethyl ester

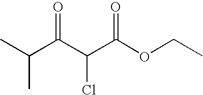

A solution of sulfuryl chloride (1.63 mL, 19.7 mmol) in toluene (5 mL) was added dropwise to a solution of 4-methyl-3-oxopentanoic acid ethyl ester (3.28 g, 19.7 mmol) in toluene (25 mL) over 10 min. The resulting mixture was stirred at rt for 18 h and then slowly quenched with water and NaHCO₃ (saturated, aqueous). The mixture was extracted with ethyl acetate, and the combined organics were dried over MgSO₄ and concentrated in vacuo to give 3.4 g (70%) of the desired product which was used without further purification. LC-MS m/z 194.1 (M+H⁺); RT 2.69 min.

### Step 2. Preparation of 2-amino-5-isopropylthiazole-4-carboxylic acid ethyl ester

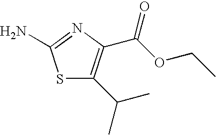

A mixture of 2-chloro-4-methyl-3-oxopentanoic acid ethyl ester (2.0 g, 7.3 mmol) and thiourea (0.43 g, 5.6 mmol) in ethanol (8 mL) was refluxed for 18 h and then concentrated in vacuo. The residue was treated with aqueous ammonia, and the resultant yellow solid was taken up in water and extracted with dichloromethane. The combined organics were dried over Na₂SO₄ and concentrated in vacuo. The solid was taken up in a small amount of dichloromethane and filtered to give 1.02 g (85%) of the desired product as a cream colored solid. LC-MS m/z 215.1 (M+H⁺); RT 1.96 min.

### Step 3. Preparation of 2-bromo-5-isopropylthiazole-4-carboxylic acid ethyl ester

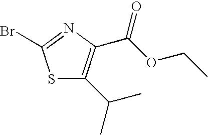

To a dark brown solution of copper (II) bromide (2.47 g, 11.1 mmol) in acetonitrile (10 mL) in a two necked flask equipped with a condenser was added tert-butylnitrite (0.63 g, 5.5 mmol) slowly at rt. The mixture was heated to 60° C., and a suspension of 2-amino-5-isopropylthiazole-4-carboxylic acid ethyl ester (0.79 g, 3.7 mmol) in acetonitrile (14 mL) was added dropwise. The mixture was then heated at 60° C. for 3 h. Upon cooling to rt, the reaction mixture was poured into 1 N NaOH (40 mL) and extracted with ethyl acetate. The combined organics were dried over Na₂SO₄, concentrated in vacuo, and purified by column chromatography (2:1 EtOAc/Hexanes) to give 0.88 g (86%) of the desired product as a yellow oil. LC-MS m/z 280 (M+H⁺); RT 3.65 min.

### Step 4. Preparation of 5-isopropyl-2-mercaptothiazole-4-carboxylic acid ethyl ester

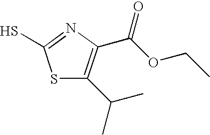

A mixture of 2-bromo-5-isopropylthiazole-4-carboxylic acid ethyl ester (0.20 g, 0.72 mmol) and thiourea (0.07 g, 0.86 mmol) in ethanol (6 mL) was heated to reflux for 2 h. Upon cooling to rt, the resulting suspension was filtered to give 0.11 g (66%) of the desired product as a yellow solid. LC-MS m/z 232.1 (M+H⁺); RT 2.72 min.

### Step 5. Preparation of 5-isopropyl-2-mercaptothiazole-4-carboxylic acid

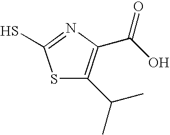

NaOH (1 N, 0.78 mL, 0.78 mmol) was added to a solution of 5-isopropyl-2-mercaptothiazole-4-carboxylic acid ethyl ester (0.09 g, 0.39 mmol) in methanol (3 mL) and water (2 mL), and the mixture was stirred at rt for 3 h. The reaction mixture was concentrated in vacuo, and the residue was acidified with 2 N HCl. The resulting suspension was filtered to give 0.045 g (57%) of the desired product as an off-white solid. ¹H NMR (400 MHz, CD₃OD) δ 3.85-4.10 (m, 1H), 1.22 (d, 6H); LC-MS m/z 204.2 (M+H⁺); RT 1.65 min.

### Example 10

### Preparation of (1-hexadecyl-1H-benzoimidazol-2-ylsulfanyl)acetic acid

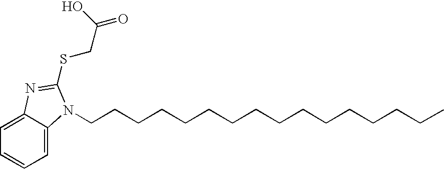

### Step 1. Preparation of (1H-benzoimidazol-2-ylsulfanyl)acetic acid ethyl ester

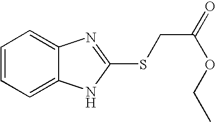

A mixture of 2-mercaptobenzimidazole (0.3 g, 2 mmol), ethyl bromoacetate (0.50 g, 2.9 mmol), and potassium carbonate (0.14 g, 0.98 mmol) in ethanol (3.1 mL) was heated to reflux for 8 h and then concentrated in vacuo. Purification by HPLC yielded 0.22 g (46%) of the desired product. LC-MS m/z 237.2 (M+H⁺); RT 1.41 min.

### Step 2. Preparation of (1-hexadecyl-1H-benzoimidazol-2-ylsulfanyl)acetic acid methyl ester

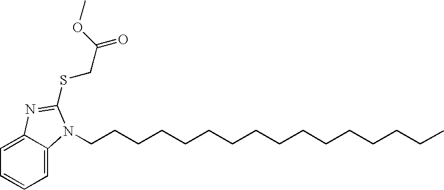

Sodium hydride (0.03 g, 0.8 mmol) was added to a solution of (1H-benzoimidazol-2-ylsulfanyl) acetic acid ethyl ester (0.21 g, 0.79 mmol) in DMF (10 mL) at 0° C., and the mixture was stirred at rt for 1 h. Hexadecylbromide (0.22 g, 0.71 mmol) was added, and the mixture was stirred at rt for 18 h. The reaction mixture was diluted with water and methanol and concentrated in vacuo. Purification by column chromatography (20% ethyl acetate in hexanes) gave 0.24 g (67%) of the desired product. LC-MS m/z 447.4 (M+H⁺); RT 5.03 min.

### Step 3. Preparation of (1-hexadecyl-1H-benzoimidazol-2-ylsulfanyl)acetic acid

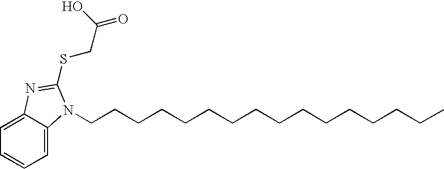

A mixture of lithium hydroxide (0.060 g, 2.4 mmol) and (1-hexadecyl-1H-benzoimidazol-2-ylsulfanyl) acetic acid methyl ester (0.21 g, 0.47 mmol) was heated to reflux for 2 h, cooled to rt, and concentrated in vacuo. The residue was taken up in water, and the suspension was acidified with 1 N HCl and filtered. The solid was collected and dried to give 0.16 g (79%) of the desired product. ¹H NMR (400 MHz, CD₃OD) δ 7.70-7.20 (m, 4H), 4.20 (t, 2H), 3.80 (s, 2H), 1.85 (m, 2H), 1.21-1.50 (m, 28H), 0.90 (t, 3H); LC-MS m/z 433.2 (M+H⁺); RT 4.72 min.

### Example 1P

### Preparation of Lithium 6-(2-tritylsulfanylethylamino)nicotinate

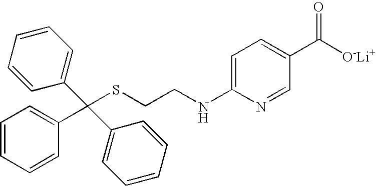

### Step 1. Preparation of 6-(2-tritylsulfanylethylamino)nicotinic acid methyl ester

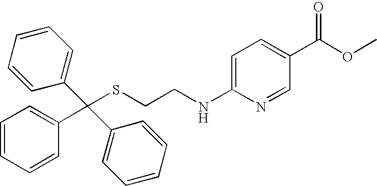

Methyl 6-chloronicotinate (0.20 g, 1.17 mmol), 2-tritylsulfanylethylamine (0.56 g, 1.75 mmol), cesium carbonate (0.95 g, 2.91 mmol), and [1,1′-bis(diphenylphosphino)ferrocene]dichloropalladium(II) complex with dichloromethane (1:1) (0.24 g, 0.29 mmol) were heated to 120° C. overnight in 1,4-dioxane (4.0 mL) and water. Upon cooling to rt, the reaction mixture was filtered through Celite®, concentrated in vacuo, and purified by Biotage column chromatography (5% EtOAc/Hexanes). This yielded 0.3442 g of a white solid. The material was recrystallized from 10% EtOAc/Hexanes, yielding 0.272 g (51%) of the desired product as a white solid. Rf=0.42 (20% EtOAc/Hexanes). ¹H NMR (400 MHz, CD₂Cl₂) δ 2.0 (bs, 1H), 2.45 (t, 2H), 3.40 (t, 2H), 3.92 (s, 3H), 7.15-7.30 (m, 10H), 7.42-7.50 (m, 6H), 8.02 (d, 1H), 8.90 (s, 1H).

### Step 2. Preparation of Lithium 6-(2-tritylsulfanylethylamino)nicotinate

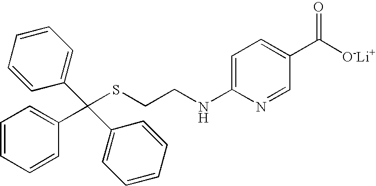

6-(2-Tritylsulfanylethylamino)nicotinic acid methyl ester (270 mg, 0.59 mmol) was suspended in THF (2 mL), MeOH (2 mL), and water (1 mL). The reaction mixture was treated with LiOH (20 mg, 0.65 mmol) and heated to 50° C. for 2 hours. Upon cooling to rt, the reaction mixture was concentrated in vacuo. Recrystallization from EtOAc/Hexanes (3:1) yielded 236 mg (89%) of the desired product as a white solid. ¹H NMR (400 MHz, DMSO-_{d6}) [symbol] 2.25 (t, 2H), 3.00 (bt, 1H), 3.28 (t, 2H), 7.00-7.40 (m, 16H), 7.90 (d, 1H), 8.72 (s, 1H).

### Example 2

### Peptide Synthesis

Peptides are synthesized with an Applied Biosystems 430A peptide synthesizer using FMOC chemistry with HBTU activation on Rink amide resin. Standard Applied Biosystems protocols are used. The peptides are cleaved with 84.6% TFA, 4.4% phenol, 4.4% water, 4.4% thioanisol, and 2.2% ethanedithiol. Peptides are precipitated from the cleavage cocktail using cold tertbutylmethyl ether. The precipitate is washed with the cold ether and dried under argon. Peptides are purified with by reversed phase C₁₈ HPLC with linear water/acetonitrile gradients containing 0.1% TFA. Peptide Identity is confirmed with MALDI and electrospray mass spectrometry and with amino acid analysis.

### Example 3

### Methods for Adding N-Terminal Modifying Compound

Peptides are synthesized with an Applied Biosystems 430A peptide synthesizer using FMOC chemistry with HBTU activation on Rink amide resin. Standard Applied Biosystems protocols are used. The N-terminal modifying compounds are coupled to the peptide as per a natural amino acid coupling during FMOC chemistry. N-terminal modifying compounds are either commercially available or synthesized as described in Example 1. In the case of amine and mercapto containing N-terminal modifying compounds, the amine and mercapto groups are protected with FMOC or trityl, respectively, during coupling to the peptide. The peptides are cleaved with 84.6% TFA, 4.4% phenol, 4.4% water, 4.4% thioanisol, and 2.2% ethanedithiol. Peptides are precipitated from the cleavage cocktail using cold tertbutylmethyl ether. The precipitate is washed with the cold ether and dried under argon. Peptides are purified with by reversed phase C18 HPLC with linear water/acetonitrile gradients containing 0.1% TFA. Peptide identity is confirmed with MALDI and electrospray mass spectrometry and with amino acid analysis.

### Example 4

### Methods for Adding C-Terminal Modifying Compound

Peptides are synthesized with an Applied Biosystems 433A peptide synthesizer using FMOC chemistry with HBTU activation on Rink amide resin. Standard Applied Biosystems protocols are used. The HBTU-activated C-terminal modifying compounds are coupled to the resin (e.g., Wang resin for producing peptides with a C-terminal modifying compound containing the free carboxylate or Rink Amide for producing amide variants) as per a natural amino acid coupling during FMOC chemistry. The peptides are then synthesized by the stepwise addition of amino acids using standard FMOC protocols. The peptides are cleaved with 84.6% TFA, 4.4% phenol, 4.4% water, 4.4% thioanisol, and 2.2% ethanedithiol. Peptides are precipitated from the cleavage cocktail using cold tertbutylmethyl ether. The precipitate is washed with the cold ether and dried under argon. Peptides are purified with by reversed phase C18 HPLC with linear water/acetonitrile gradients containing 0.1% TFA. Peptide identity is confirmed with MALDI and electrospray mass spectrometry and with amino acid analysis.

### Example 5

### Preparation of Pegylated Peptides

PEG derivatives are prepared by incubating methoxypolyethlene glycols derivatized with maledimide for coupling to the mercapto moiety of the N-terminal modifying group. mPEG-MAL or mPEG2-MAL products supplied by Nektar Therapeutics (Huntsville, Ala., USA) or GLE-200MA or GLE-400MA products supplied by NOF (Toyko, Japan) are used. Coupling reactions are performed by incubating the peptide and a two-fold molar excess of maleimide-PEG in 50 mM Tris, pH 7 at rt for 2-12 h. The peptide concentration may be 1 mg/ml or less. Underivatized peptides and PEG are purified from the PEGylated peptide with cation exchange chromatography and dialysis or by reversed phase C₁₈ HPLC. The purified PEG-peptide conjugate is then freeze dried.

### Example 6

### Preparation of Fatty-Acid Derivatized Peptides

The fatty acid (palmitate) derivatives of amine containing N-terminal modifying compounds are prepared as N-terminal modified peptides as described in Example 3 except that prior to deprotection and cleavage the FMOC protecting group of the amine moiety of the N-terminal modifying group was selectively removed with 0.1% TFA and derivatized with palmitic acid using the same conditions as for a normal amino acid coupling.

The fatty acid derivative can also be prepared as described in Example 3 using 1-hexadecyl-1H-benzoimidazol-2-ylsulfanyl)acetic acid as the N-terminal modifying group, which was synthesized as described in Example 1.

### Example 7

### Pharmaceutical Composition—IV and SC Formulations

A sterile IV injectable formulation is prepared with 4 mg of a peptide of Formula (I), or a derivatized polypetide having equivalent of 4 mg peptide content, and 1 L sterile saline, using any manufacturing process well known in the art. Higher concentrations of peptide may be used for SC formulation. In the case of the peptide of Formula (I), or a derivatized peptide, 4 mg is dissolved in 100 mL saline or DMSO and sterile vials after aseptic filtration, are filled with the composition.

### Example 8

### Mass Spectrometric Analysis of Peptides

Forty pmol/2 μl aliquots of peptides are diluted up to 10 μl with water. The HEPES buffer is removed by application of 50% of the sample (20 pmol/5 μl) to a conditioned Millipore C18 ZipTip, as per manufacturers instructions. Samples are eluted from the zipTip with matrix (10 mg/ml alpha-cyanohydroxycinnamic acid in 50% ACN, 0.1% TFA) directly onto the MALDI plate. Samples are analyzed on an Applied Biosystems Voyager DE-PRO MALDI operated in the reflector ion mode. Data is collected in the 500-4000 Da range and resulting masses were compared to those expected by manual calculation.

### Example 9

### Edman Analysis of Peptides

Peptide samples are supplied for Edman degradation at 1 nmol/10 μl in 10 mM HEPES, pH 7.4, 5% TFA. Prior to Edman analysis, the HEPES buffer salt is removed by using an Applied Biosystems ProSorb sample cartridge as per manufacturers instructions. Briefly, the sample is applied to a PVDF membrane and washed with 0.1% TFA, then the membrane is removed and inserted into the protein sequencer for Edman degradation. Edman degradation is carried out on an Applied Biosystems Procise 494HT protein sequencing system using the pulsed-liquid method according to manufacturer instructions. Sequences are read manually.

### Example 10

### Stability of Peptides

The formulations described in Example 4 are placed in constant stability chamber. Peptides are also analyzed for stability to degradation in solutions of DPPIV and in plasma. Samples are removed periodically for analysis by capillary electrophoresis, mass spectrometry, Edman degradation, ELISA, and assays of peptide activity, which are sensitive methods to detect degradation of peptide. The area of various peaks is summed and the area for peak of the parent peptide is divided by the total peak area. The quotient is the % purity. Since there are impurities present in the fresh peptide, the purity change is normalized by dividing the purity at different time point by the initial purity. For stability to DPPIV and plasma, peptides at 20 pmol/μl were incubated at 37° C. in the presence of 300 pM DPPIV in 100 mM HEPES, pH 7.4. At various timepoints, the reaction (2 μl aliquot) is terminated by addition of 1 μM DPPIV inhibitor and freezing. For MALDI mass spectrometric analysis, T=0 hr, 1 hr, 5 hr, and 24 hr timepoints are evaluated. The results are plotted as percent intact peptide or peptide derivative as compared to degradation products.

### Example 11

### Binding of Peptides to PACAP1 and VPAC1 and 2 Receptors

CHO cells overexpressing the PAC1, VPAC1, and VPAC2 receptors are grown to confluency, scraped from their flasks, and pelleted in a soft spin in 50 ml tubes. The pellets are resuspended in a Tris based homogenization buffer and homogenized in a Dounce tissue grinder with 30-40 manual strokes on ice. The suspension is spun in an ultracentrifuge which pellets the membranes. This pellet is resuspended in a small amount of homogenization buffer and a protein concentration is determined through the use of a BCA kit from Pierce.

A binding reaction containing 10 μg membrane protein, 0.1 nM ¹²⁵I-PACAP-27, and a dose curve of peptide to be tested is incubated in a 96-well plate at 37° C. for 20 min. The reaction is stopped by placement of the plate on ice for 20 min. The reaction is added to a filter plate preincubated with 0.1% PEI to avoid non-specific binding, processed on a vacuum manifold, and washed several times with a BSA based wash solution. The filter plate is dried, scintillant added, and read on a MicroBeta counter. The data is analyzed and presented in Prism graphs.

### Example 12

### Elevation of cAMP in Response to Peptides

CHO cells expressing the VPAC2 peptide are plated in 96-well plates at 8×10⁴ cells/well and grown at 37° C. for 24 hours in αMEM, nucleosides, glutamine (Gibco/BRL, Rockville, Md.), 5% FBS, 100 μg/mL Pen/Strep, 0.4 mg/mL hygromycin, and 1.5 mg/mL Geneticin (Gibco/BRL). The media is removed, and the plates are washed with PBS. The cells are incubated with a peptide (in 10 mM Hepes, 150 mM NaCL, 5 mM KCL, 2.5 mM CaCl₂, 1.2 mM KH₂PO₄, 1.2 mM MgSO₄, 25 mM NaHCO₃ (pH 7.4) with 1% BSA and 100 μM IBMX) for 15 min at 37° C. Cyclic AMP in the cell extracts is quantitated using the cAMP SPA direct screening assay system (Amersham Pharmacia Biotech Inc., Piscataway, N.J.,). The amount of cAMP present in the lysates is determined following instructions provided with this kit. The amount of cAMP (in pmol) produced at each concentration of peptide is plotted and analyzed by nonlinear regression using Prizm software to determine the EC₅₀ for each peptide.

Alternatively, the elevation of cAMP in response to receptor activation can be measured in a reporter cell line, such as CHO, which not only expresses the desired receptor but which also expresses a reporter, such as luciferase, linked to a cAMP response element (CRE). Such cell are plated in 96-well plates at 10⁴ cells per well and grown at 37° C. for 48 hours in αMEM, nucleosides, glutamine (Gibco/BRL, Rockville, Md.), 5% FBS, 100 μg/mL Pen/Strep, 0.4 mg/mL hygromycin, and 1.5 mg/mL Geneticin (Gibco/BRL). The cells are then incubated with peptide for 6 hours, the media removed, and Bright-Glo reagent (Promega) added. The signal is detected using a scintillation counter.

### Example 13

### Insulin Secretion from Dispersed Rat Islet Cells

Insulin secretion of dispersed rat islets mediated by a number of peptides of the present invention is measured as follows. Islets of Langerhans, isolated from SD rats (200-250 g), are digested using collagenase. The dispersed islet cells are treated with trypsin, seeded into 96 V-bottom plates, and pelleted. The cells are then cultured overnight in media with or without peptides of this invention. The media is aspirated, and the cells are pre-incubated with Krebs-Ringer-HEPES buffer containing 3 mM glucose for 30 minutes at 37° C. The pre-incubation buffer is removed, and the cells are incubated at 37° C. with Krebs-Ringer-HEPES buffer containing the appropriate glucose concentration (e.g., 8 mM) with or without peptides for an appropriate time. A portion of the supernatant is removed and its insulin content is measured by SPA. The results are expressed as “fold over control” (FOC).

### Example 14

### Generation of Peptide Specific Antibodies and Peptide Measurement by ELISA

Polyclonal antibodies specific to the peptides of the present invention are generated by synthesizing a specific fragment of a peptide of this invention using an ABI 433A peptide synthesizer. The peptide is then cleaved from the resin, and purified on a Beckman System Gold Analytical and Preparative HPLC system. A Perspective MALDI mass spectrophotometer system is used to identify the correct product. The peptide is dried using a lyophilizer. The peptide (2 mg) is then conjugated to keyhole limpet hemocyanin (KLH) via the free sulphydryl group on the Cys.

Female New Zealand White rabbits are immunized on Day 0, 14, 35, 56, and 77. On Day 0, each rabbit is injected subcutaneous with 250 μg peptide and complete Freund's adjuvant. Subsequent immunizations utilize 125 μg peptide per rabbit. Bleeds are started on Day 21 and continued at 21-day intervals thereafter. Purification of anti-peptide antibodies is performed by passing the crude serum over a specific peptide affinity purification column. The antibody titer is determined by ELISA.

A 96-well Immulon 4HBX plate is coated with a C-terminal F(ab) antibody, specific to the peptides of the present invention, and allowed to incubate overnight at 4° C. The plate is then blocked to prevent non-specific binding. Then, peptide standards (2500 ng/mL-160 pg/mL) are diluted in 33% plasma and the samples are diluted 1:3 in buffer followed by incubation for 1.5 h at rt. After washing, a polyclonal N-terminal antibody specific to the peptides of this invention is incubated on the plate for 1 h. This is followed by the addition of horseradish peroxidase (HRP)-donkey-anti-rabbit antibody and the samples and standards are incubated for another hour. Detection is assessed following incubation with 3,3′,5,5′-tetramethylbenzidine (TMB) solution, and the plate is read at OD₄₅₀.

Alternatively, the 96-well Immulon 4HBX plate is coated with a polyclonal N-terminal antibody, specific to the peptides of the present invention, and allowed to incubate overnight at 4° C. The plate is then blocked to prevent non-specific binding. Then, peptide standards (2500 ng/mL-160 pg/mL) are diluted in 50% plasma and the samples are diluted 1:2 in buffer followed by incubation for 1.5 h at rt. After washing, a monoclonal anti-PEG antibody specific to the peptides of this invention is incubated on the plate for one hour. This was followed by the addition of horseradish peroxidase (HRP)—anti-mouse antibody and the samples and standards are incubated for another hour. Detection is assessed following incubation with 3,3′,5,5′-tetramethylbenzidine (TMB) solution, and the plate is read at OD₄₅₀.

### Example 15

### Phamacokinetics of Peptides Following IV and Subcutaneous Dosing

Plasma samples are transferred to a microcentrifuge tube and an equal volume of acetonitrile is added to the sample (a 50% final concentration). The sample is vigorously vortexed for about 5 min and allowed to sit on ice for 10 min. The sample is again vortexed for about 1 min, and then centrifuged for 30 min in a microcentrifuge (4° C.) at maximum (about 15,000×g).

Following centrifugation, the aqueous phase is carefully transferred to a clean centrifuge tube, and the sample is centrifuged for 5 min in a microcentrifuge (4° C.) at maximum speed (about 15,000×g). The extracted sample is dried under vacuum using a Speed Vac SC110 (Savant) with a medium heat setting until dry. The sample is resuspended in an appropriate volume of sterile water and is maintained at 4° C. The sample is then sonicated in a sonibath for 10 min at rt prior to analysis.

### Example 16

### Effect of Peptides on Intraperitoneal Glucose Tolerance in Rats

The in vivo activity of the peptides of this invention when administered subcutaneously is examined in rats. Rats fasted overnight are given a subcutaneous injection of control or peptide (1-100 μg/kg). Three hours later, basal blood glucose is measured, and the rats are given 2 g/kg of glucose intraperitoneally. Blood glucose is measured again after 15, 30, and 60 min.

Demonstration of the activity of the peptides of the present invention may be accomplished through in vitro, ex vivo, and in vivo assays that are well known in the art. For example, to demonstrate the efficacy of a pharmaceutical agent for the treatment of diabetes and related disorders such as Syndrome X, impaired glucose tolerance, impaired fasting glucose, and hyperinsulinemia; atherosclerotic disease and related disorders such as hypertriglyceridemia and hypercholesteremia; and obesity, the following assays may be used.

### Example 17

### Method for Measuring Blood Glucose Levels

db/db mice (obtained from Jackson Laboratories, Bar Harbor, Me.) are bled (by either eye or tail vein) and grouped according to equivalent mean blood glucose levels. They are dosed with the test peptide for 14 days. At this point, the animals are bled again by eye or tail vein and blood glucose levels were determined. In each case, glucose levels are measured with a Glucometer Elite XL (Bayer Corporation, Elkhart, Ind.).

### Example 18

### Method for Measuring an Effect on Cardiovascular Parameters

Cardiovascular parameters (e.g., heart rate and blood pressure) are also evaluated. SHR rats are dosed with vehicle or test peptide for 2 weeks. Blood pressure and heart rate are determined using a tail-cuff method as described by Grinsell, et al., (Am. J. Hypertens. 13:370-375, 2000). In monkeys, blood pressure and heart rate are monitored as described by Shen, et al., (J. Pharmacol. Exp. Therap. 278:1435-1443, 1996).

### Example 19

### Method for Measuring Triglyceride Levels

hApoA1 mice (obtained from Jackson Laboratories, Bar Harbor, Me.) are bled (by either eye or tall vein) and grouped according to equivalent mean serum triglyceride levels. They are dosed with the test peptide for 8 days. The animals are then bled again by eye or tail vein, and serum triglyceride levels are determined. In each case, triglyceride levels are measured using a Technicon Axon Autoanalyzer (Bayer Corporation, Tarrytown, N.Y.).

### Example 20

### Method for Measuring HDL-Cholesterol Levels

To determine plasma HDL-cholesterol levels, hApoA1 mice are bled and grouped with equivalent mean plasma HDL-cholesterol levels. The mice are dosed with vehicle or test peptide for 7 days, and then bled again on day 8. Plasma is analyzed for HDL-cholesterol using the Synchron Clinical System (CX4) (Beckman Coulter, Fullerton, Calif.).

### Example 21

### Method for Measuring Total Cholesterol, HDL-Cholesterol, Triglycerides, and Glucose Levels

In another in vivo assay, obese monkeys are bled, then dosed with vehicle or test peptide for 4 weeks, and then bled again. Serum is analyzed for total cholesterol, HDL-cholesterol, triglycerides, and glucose using the Synchron Clinical System (CX4) (Beckman Coulter, Fullerton, Calif.). Lipoprotein subclass analysis is performed by NMR spectroscopy as described by Oliver, et al., (Proc. Natl. Acad. Sci. USA 98:5306-5311, 2001).

All publications and patents mentioned in the above specification are incorporated herein by reference. Various modifications and variations of the described compositions and methods of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the above-described modes for carrying out the invention which are obvious to those skilled in the field of biochemistry or related fields are intended to be within the scope of the following claims. Those skilled in the art will recognize, or be able to ascertain using no more than routine experimentation, many equivalents to the specific embodiments of the invention described herein. Such equivalents are intended to be encompassed by the following claims.

TABLE 1

| SEQ ID | |
|---|---|
| NO | Sequence |
| 2 | HSDAVFTDQYTRLRKQVAAKKYLQSIKQKRY |
| 3 | HSDAVFTDQYTRLRKQMAAKKYLQSIKQKRY |
| 4 | HSDAVFTDQYTRLRKQVAAKKYLQSIKQK |
| 5 | HTEAVFTDQYTRLRKQVAAKKYLQSIKQKRY |
| 6 | HSDAVFTDQYTRLRKQLAVKKYLQDIKQGGT |
| 7 | HSDAVFTDQYTRLRKQMAAKKYLQSIKQKR |
| 8 | HSDAVFTDQYTRLRKQLAAKKYLQTIKQKRY |
| 9 | HSDAVFTDQYTRLRKQMAAKKYLQTIKQKRY |
| 10 | HSDAVFTDQYTRLRKQMAAHKYLQSIKQKRY |
| 11 | HSDAVFTDQYTRLRKQMAAKHYLQSIKQKRY |
| 12 | HSDAVFTDQYTRLRKQMAGKKYLQSIKQKR |
| 13 | HSDAVFTDQYTRLRKQMAKKKYLQSIKQKR |
| 14 | HSDAVFTDQYTRLRKQMARKKYLQSIKQKR |
| 15 | HSDAVFTDQYTRLRKQMASKKYLQSIKQKR |
| 16 | HSDAVFTDQYTRLRKQMAAKKYLQSIPQKR |
| 17 | HSDAVFTDQYTRLRKQMAAKKYLQSIQQKR |
| 18 | HSDAVFTDQYTRLRKQMAAKKYLQSIRQKR |
| 19 | HSDAVFTDQYTRLRKQMAAKKYLQSIKQRR |
| 20 | HSDAVFTDQYTRLRKQMAAKKYLQSIKQKA |
| 21 | HSDAVFTDQYTRLRKQMAAKKYLQSIKQKF |
| 22 | HSDAVFTDQYTRLRKQMAAKKYLQSIKQKH |
| 23 | HSDAVFTDQYTRLRKQMAAKKYLQSIKQKI |
| 24 | HSDAVFTDQYTRLRKQMAAKKYLQSIKQKK |
| 25 | HSDAVFTDQYTRLRKQMAAKKYLQSIKQKL |
| 26 | HSDAVFTDQYTRLRKQMAAKKYLQSIKQKM |
| 27 | HSDAVFTDQYTRLRKQMAAKKYLQSIKQKP |
| 28 | HSDAVFTDQYTRLRKQMAAKKYLQSIKQKQ |
| 29 | HSDAVFTDQYTRLRKQMAAKKYLQSIKQKS |
| 30 | HSDAVFTDQYTRLRKQMAAKKYLQSIKQKT |
| 31 | HSDAVFTDQYTRLRKQMAAKKYLQSIKQKV |
| 32 | HSDAVFTDQYTRLRKQMAAKKYLQSIKQKW |
| 33 | HSDAVFTDQYTRLRKQMAAKKYLQSIKQKY |
| 34 | HSDAVFTDQYTRLRKQMAGKKYLQSIKQRI |
| 35 | HSDAVFTDQYTRLRKQMAKKKYLQSIKQRI |
| 36 | HSDAVFTDQYTRLRKQMASKKYLQSIKQRI |
| 37 | HSDAVFTDQYTRLRKQMAAKKYLQSIPQRI |
| 38 | HSDAVFTDQYTRLRKQMASKKYLQSIRQRI |
| 39 | HSDAVFTDNYTRLRKQVAAKKYLQSIKQKRY |
| 40 | HSDAVFTDNYTRLRKQMAAKKYLQSIKQKRY |
| 41 | HSDAVFTDNYTRLRKQVAAKKYLQSIKQK |
| 42 | HTEAVFTDNYTRLRKQVAAKKYLQSIKQKRY |
| 43 | HSDAVFTDNYTRLRKQLAVKKYLQDIKQGGT |
| 44 | HSDAVFTDNYTRLRKQMAAKKYLQSIKQKR |
| 45 | HSDAVFTDNYTRLRKQLAAKKYLQTIKQKRY |
| 46 | HSDAVFTDNYTRLRKQMAAKKYLQTIKQKRY |
| 47 | HSDAVFTDNYTRLRKQMAAHKYLQSIKQKRY |
| 48 | HSDAVFTDNYTRLRKQMAAKHYLQSIKQKRY |
| 49 | HSDAVFTDNYTRLRKQMAGKKYLQSIKQKR |
| 50 | HSDAVFTDNYTRLRKQMAKKKYLQSIKQKR |
| 51 | HSDAVFTDNYTRLRKQMARKKYLQSIKQKR |
| 52 | HSDAVFTDNYTRLRKQMASKKYLQSIKQKR |
| 53 | HSDAVFTDNYTRLRKQMAAKKYLQSIPQKR |
| 54 | HSDAVFTDNYTRLRKQMAAKKYLQSIQQKR |
| 55 | HSDAVFTDNYTRLRKQMAAKKYLQSIRQKR |
| 56 | HSDAVFTDNYTRLRKQMAAKKYLQSIKQRR |
| 57 | HSDAVFTDNYTRLRKQMAAKKYLQSIKQKA |
| 58 | HSDAVFTDNYTRLRKQMAAKKYLQSIKQKF |
| 59 | HSDAVFTDNYTRLRKQMAAKKYLQSIKQKH |
| 60 | HSDAVFTDNYTRLRKQMAAKKYLQSIKQKI |
| 61 | HSDAVFTDNYTRLRKQMAAKKYLQSIKQKK |
| 62 | HSDAVFTDNYTRLRKQMAAKKYLQSIKQKL |
| 63 | HSDAVFTDNYTRLRKQMAAKKYLQSIKQKM |
| 64 | HSDAVFTDNYTRLRKQMAAKKYLQSIKQKP |
| 65 | HSDAVFTDNYTRLRKQMAAKKYLQSIKQKQ |
| 66 | HSDAVFTDNYTRLRKQMAAKKYLQSIKQKS |
| 67 | HSDAVFTDNYTRLRKQMAAKKYLQSIKQKT |
| 68 | HSDAVFTDNYTRLRKQMAAKKYLQSIKQKV |
| 69 | HSDAVFTDNYTRLRKQMAAKKYLQSIKQKW |
| 70 | HSDAVFTDNYTRLRKQMAAKKYLQSIKQKY |
| 71 | HSDAVFTDNYTRLRKQMAGKKYLQSIKQRI |
| 72 | HSDAVFTDNYTRLRKQMAKKKYLQSIKQRI |
| 73 | HSDAVFTDNYTRLRKQMASKKYLQSIKQRI |
| 74 | HSDAVFTDNYTRLRKQMAAKKYLQSIPQRI |
| 75 | HSDAVFTDNYTRLRKQMASKKYLQSIRQRI |
| 76 | HSDAVFTDQYTRLRKQVAAKKYLQSIKNKRY |
| 77 | HSDAVFTDQYTRLRKQMAAKKYLQSIKNKRY |
| 78 | HSDAVFTDQYTRLRKQVAAKKYLQSIKNK |
| 79 | HTEAVFTDQYTRLRKQVAAKKYLQSIKNKRY |
| 80 | HSDAVFTDQYTRLRKQLAVKKYLQDIKNGGT |
| 81 | HSDAVFTDQYTRLRKQMAAKKYLQSIKNKR |
| 82 | HSDAVFTDQYTRLRKQLAAKKYLQTIKNKRY |
| 83 | HSDAVFTDQYTRLRKQMAAKKYLQTIKNKRY |
| 84 | HSDAVFTDQYTRLRKQMAAHKYLQSIKNKRY |
| 85 | HSDAVFTDQYTRLRKQMAAKHYLQSIKNKRY |
| 86 | HSDAVFTDQYTRLRKQMAGKKYLQSIKNKR |
| 87 | HSDAVFTDQYTRLRKQMAKKKYLQSIKNKR |
| 88 | HSDAVFTDQYTRLRKQMARKKYLQSIKNKR |
| 89 | HSDAVFTDQYTRLRKQMASKKYLQSIKNKR |
| 90 | HSDAVFTDQYTRLRKQMAAKKYLQSIPNKR |
| 91 | HSDAVFTDQYTRLRKQMAAKKYLQSIQNKR |
| 92 | HSDAVFTDQYTRLRKQMAAKKYLQSIRNKR |
| 93 | HSDAVFTDQYTRLRKQMAAKKYLQSIKNRR |
| 94 | HSDAVFTDQYTRLRKQMAAKKYLQSIKNKA |
| 95 | HSDAVFTDQYTRLRKQMAAKKYLQSIKNKF |
| 96 | HSDAVFTDQYTRLRKQMAAKKYLQSIKNKH |
| 97 | HSDAVFTDQYTRLRKQMAAKKYLQSIKNKI |
| 98 | HSDAVFTDQYTRLRKQMAAKKYLQSIKNKK |
| 99 | HSDAVFTDQYTRLRKQMAAKKYLQSIKNKL |
| 100 | HSDAVFTDQYTRLRKQMAAKKYLQSIKNKM |
| 101 | HSDAVFTDQYTRLRKQMAAKKYLQSIKNKP |
| 102 | HSDAVFTDQYTRLRKQMAAKKYLQSIKNKQ |
| 103 | HSDAVFTDQYTRLRKQMAAKKYLQSIKNKS |
| 104 | HSDAVFTDQYTRLRKQMAAKKYLQSIKNKT |
| 105 | HSDAVFTDQYTRLRKQMAAKKYLQSIKNKV |
| 106 | HSDAVFTDQYTRLRKQMAAKKYLQSIKNKW |
| 107 | HSDAVFTDQYTRLRKQMAAKKYLQSIKNKY |
| 108 | HSDAVFTDQYTRLRKQMAGKKYLQSIKNRI |
| 109 | HSDAVFTDQYTRLRKQMAKKKYLQSIKNRI |
| 110 | HSDAVFTDQYTRLRKQMASKKYLQSIKNRI |
| 111 | HSDAVFTDQYTRLRKQMAAKKYLQSIPNRI |
| 112 | HSDAVFTDQYTRLRKQMASKKYLQSIRNRI |
| 113 | HSDAVFTDQYTRLRKQVAAKKYLQSIKQKRYC |
| 114 | HSDAVFTDQYTRLRKQMAAKKYLQSIKQKRYC |
| 115 | HSDAVFTDQYTRLRKQVAAKKYLQSIKQKC |
| 116 | HTEAVFTDQYTRLRKQVAAKKYLQSIKQKRYC |
| 117 | HSDAVFTDQYTRLRKQLAVKKYLQDIKQGGTC |
| 118 | HSDAVFTDQYTRLRKQMAAKKYLQSIKQKRC |
| 119 | HSDAVFTDQYTRLRKQLAAKKYLQTIKQKRYC |
| 120 | HSDAVFTDQYTRLRKQMAAKKYLQTIKQKRYC |
| 121 | HSDAVFTDQYTRLRKQMAAHKYLQSIKQKRYC |
| 122 | HSDAVFTDQYTRLRKQMAAKHYLQSIKQKRYC |
| 123 | HSDAVFTDQYTRLRKQMAGKKYLQSIKQKRC |
| 124 | HSDAVFTDQYTRLRKQMAKKKYLQSIKQKRC |
| 125 | HSDAVFTDQYTRLRKQMARKKYLQSIKQKRC |
| 126 | HSDAVFTDQYTRLRKQMASKKYLQSIKQKRC |
| 127 | HSDAVFTDQYTRLRKQMAAKKYLQSIPQKRC |
| 128 | HSDAVFTDQYTRLRKQMAAKKYLQSIQQKRC |
| 129 | HSDAVFTDQYTRLRKQMAAKKYLQSIRQKRC |
| 130 | HSDAVFTDQYTRLRKQMAAKKYLQSIKQRRC |
| 131 | HSDAVFTDQYTRLRKQMAAKKYLQSIKQKAC |
| 132 | HSDAVFTDQYTRLRKQMAAKKYLQSIKQKFC |
| 133 | HSDAVFTDQYTRLRKQMAAKKYLQSIKQKHC |
| 134 | HSDAVFTDQYTRLRKQMAAKKYLQSIKQKIC |
| 135 | HSDAVFTDQYTRLRKQMAAKKYLQSIKQKKC |
| 136 | HSDAVFTDQYTRLRKQMAAKKYLQSIKQKLC |
| 137 | HSDAVFTDQYTRLRKQMAAKKYLQSIKQKMC |
| 138 | HSDAVFTDQYTRLRKQMAAKKYLQSIKQKPC |
| 139 | HSDAVFTDQYTRLRKQMAAKKYLQSIKQKQC |
| 140 | HSDAVFTDQYTRLRKQMAAKKYLQSIKQKSC |
| 141 | HSDAVFTDQYTRLRKQMAAKKYLQSIKQKTC |
| 142 | HSDAVFTDQYTRLRKQMAAKKYLQSIKQKVC |
| 143 | HSDAVFTDQYTRLRKQMAAKKYLQSIKQKWC |
| 144 | HSDAVFTDQYTRLRKQMAAKKYLQSIKQKYC |
| 145 | HSDAVFTDQYTRLRKQMAGKKYLQSIKQRIC |
| 146 | HSDAVFTDQYTRLRKQMAKKKYLQSIKQRIC |
| 147 | HSDAVFTDQYTRLRKQMASKKYLQSIKQRIC |
| 148 | HSDAVFTDQYTRLRKQMAAKKYLQSIPQRIC |
| 149 | HSDAVFTDQYTRLRKQMASKKYLQSIRQRIC |
| 150 | HSDGIFTDSYSRYRKQMAVKKYLAAVLGKRYKQRVKNK |
| | (PACAP38) |
| 151 | HSDGIFTDSYSRYRKQMAVKKYLAAVL (PACAP27) |
| 152 | HSDAVFTDNYTRLRKQMAVKKYLNSILN (VIP) |
| 153 | HSDAVFTDQYTRLRKQVAAKKYLQSIKQKRY |
| 154 | HTDAVFTDQYTRLRKQVAAKKYLQSIKQKRY |
| 155 | HSDAVFTDQYTRLRKQVAAKKYLQSIKQKRYC |
| 156 | HTDAVFTDQYTRLRKQVAAKKYLQSIKQKRYC |

TABLE 2

| PEG reagent | Structure |
|---|---|
| Linear PEG (e.g., Sunbright ME- 200MA) | 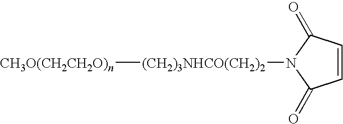 |
| Linear PEG mPEG-MAL (e.g., Nektar 2D2M0H01 and 2D2M0P01) | 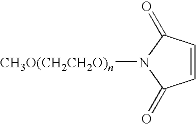 |
| Branched PEG mPEG2-MAL (e.g., Nektar 2D3X0T01) | 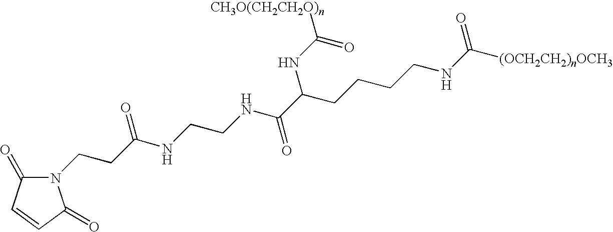 |
| Branched PEG (e.g., NOF GL2-400MA) | 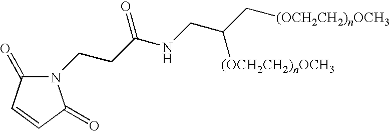 |
| Branched PEG (e.g., NOF GL2- 400MA2) | 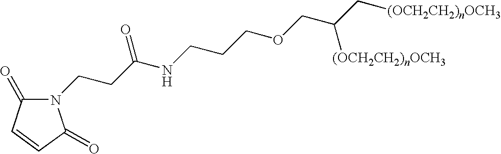 |

1. A peptide of Formula (I)
Z1-A1-A2-A3-A4-A5-Phe-Thr-A8-A9-A10-A11-A12-A13-Arg-A15-A16-A17-Ala-
A19-A20-A21-Tyr-Leu-A24-A25-A26-A27-A28-A29-A30-A31-A32-A33-A34-A35-
A36-A37-A38-A39-A40-Z2 (SEQ ID NO: 1)
wherein
A1 is His, Ala;
A2 is Ser, Thr, Ala;
A3 is Asp, Glu;
A4 is Ala, Gly;
A5 is Val, Ile;
A8 is Asp, Glu, Ala;
A9 is Gln, Asn, Ser, Ala;
A11 is Thr, Ser;
A12 is Arg, Lys;
A13 is Leu, Tyr;
A15 is Lys, Ala;
A16 is Gln, Ala;
A17 is Val, Met, Leu, Nle, Ala;
A19 is Ala, Val, Gly, Lys, Arg, Ser, Glu, Phe, Ile, Leu, Met, Thr, Trp;
A20 is Lys, His;
A21 is Lys, His, Ala;
A24 is Gln, Asn, Ala;
A25 is Ser, Asp, Thr, Ala;
A26 is Ile, Val, Leu, Ala;
A27 is any amino acid;
A28 is Gln, Asn, Gly, Ala, Lys;
A29 is Lys, Gly, Arg, Cys, Ala, Asp, Glu, His, Ile, Leu, Met, Asn, Pro, Gln, Arg, Ser, Thr or deleted;
A30 is any amino acid or deleted;
A31 is Tyr, Thr, Cys or deleted;
A32 is Lys, Cys, Lys-X, Cys-PEG or deleted;
A33 is Gln, Lys, Cys, Lys-X, Cys-PEG or deleted;
A34 is Arg, Lys, Cys, Lys-X, Cys-PEG or deleted;
A35 is Val, Lys, Cys, Lys-X, Cys-PEG or deleted;
A36 is Lys, Cys, Lys-X, Cys-PEG or deleted;
A37 is Asn, Lys, Cys, Lys-X, Cys-PEG or deleted;
A38 is Lys, Cys, Lys-X, Cys-PEG or deleted; and
A39 is Lys, Cys, Lys-X, Cys-PEG or deleted
Lys-X is Lys modified at N^{ε} with a fatty acid
Z1 is selected from 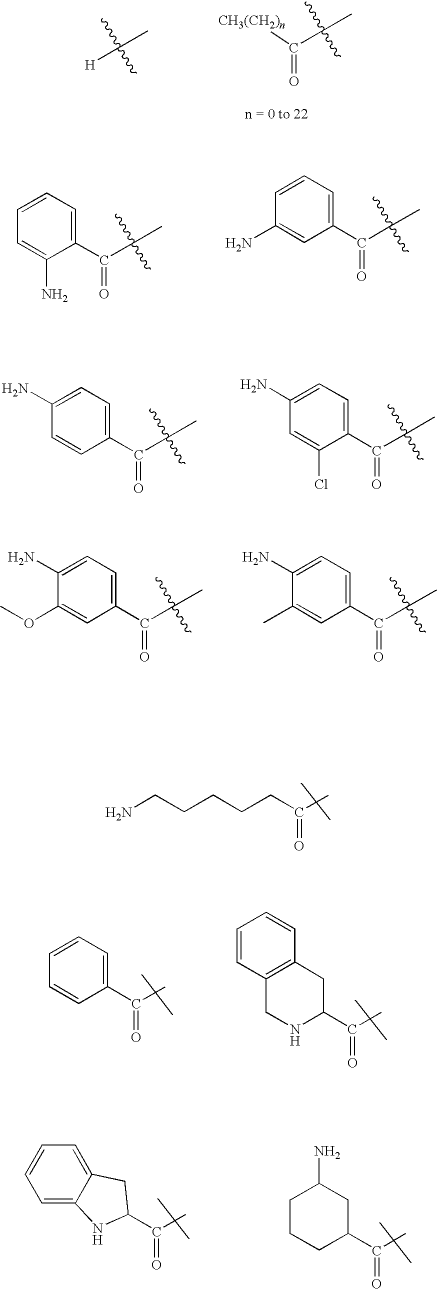 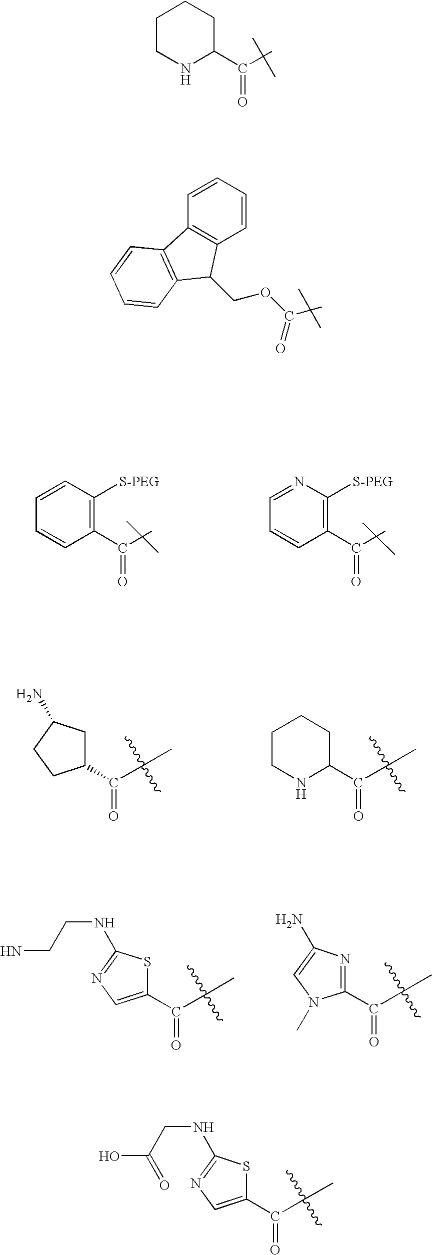 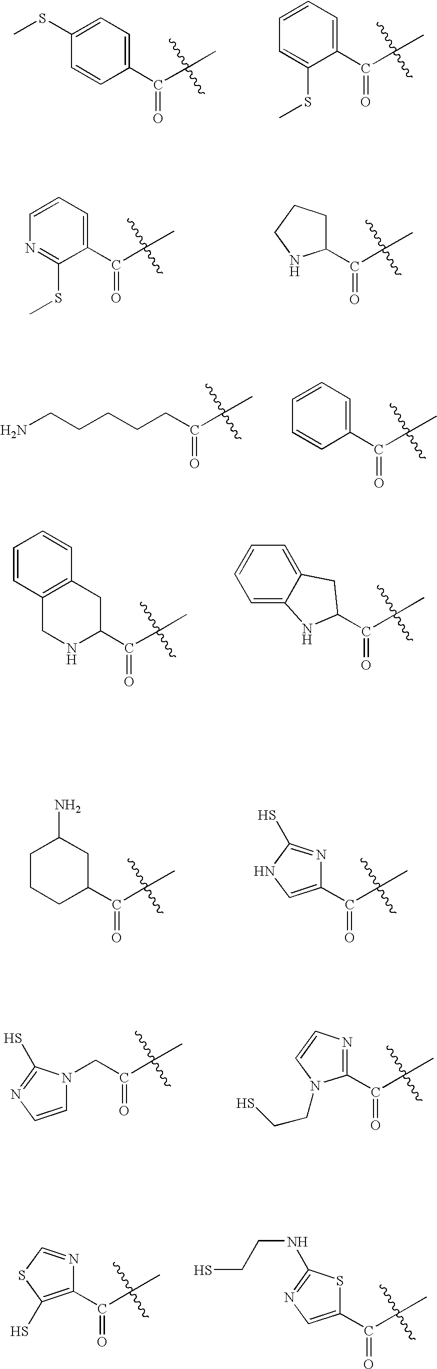 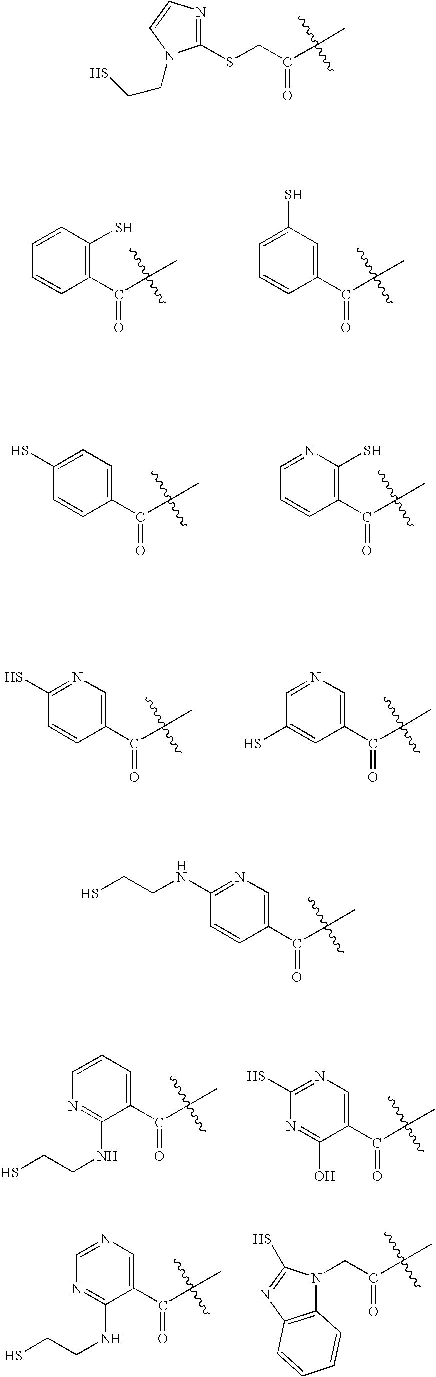 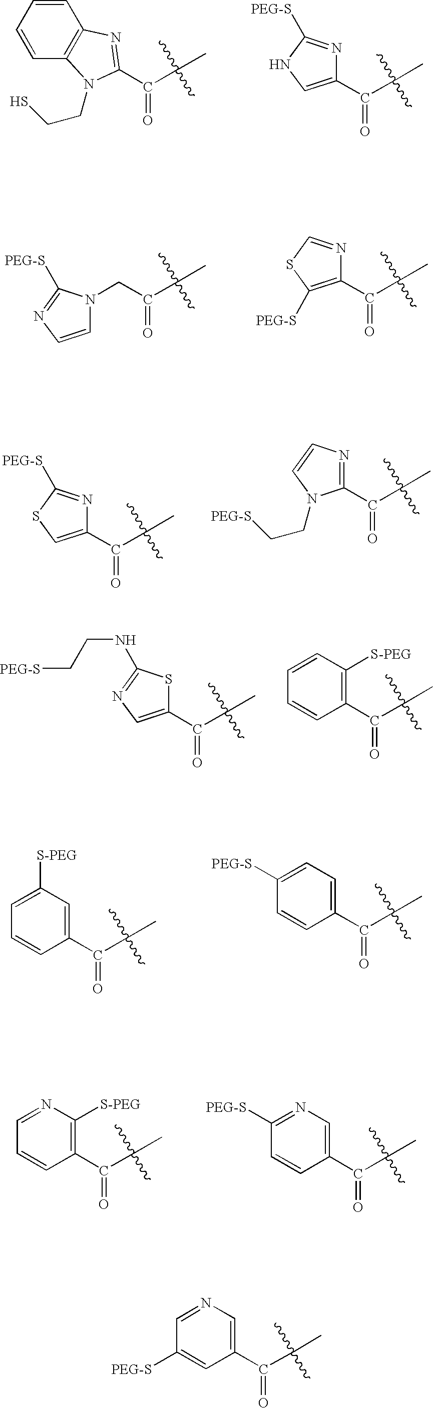 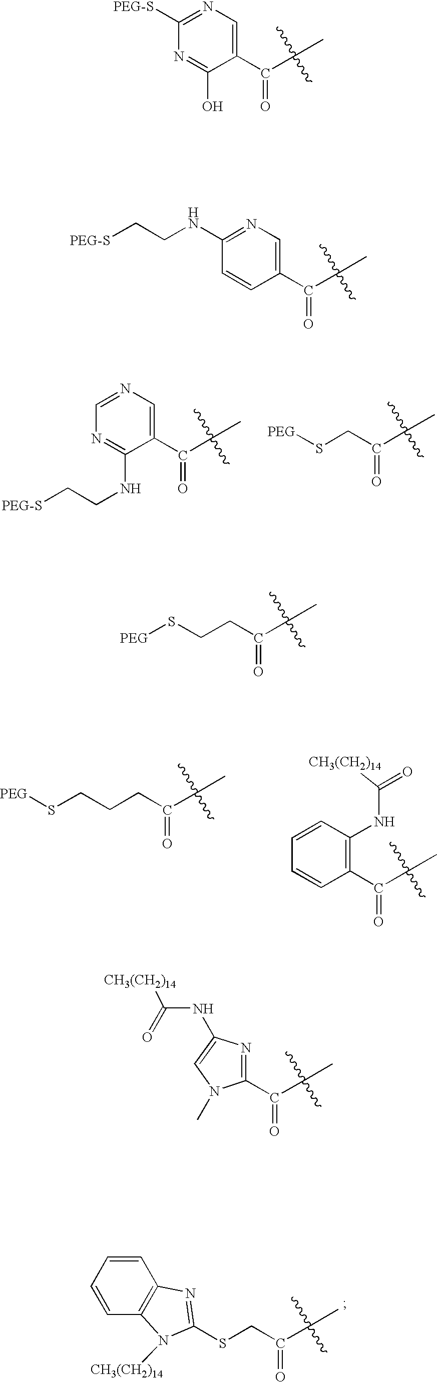
and
Z2 is selected from

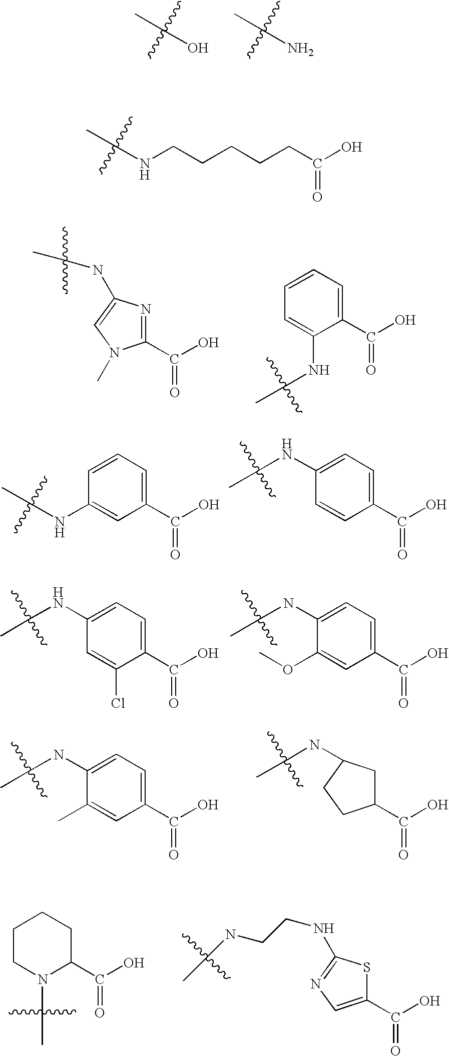

2. The peptide of claim 1, wherein said peptide is selected from SEQ ID NO: 1-156.

3. The peptide of claim 2, wherein said peptide is selected from SEQ ID NO: 1-111.

4. The peptide of claim 2, wherein said peptide is selected from SEQ ID NO: 112-156.

5. The peptide of claim 2, wherein said peptide is selected from SEQ ID NO: 2, 8, 9, 23, 34, 52, 67, 89, 103, 104, 113, 118, 133, 137, 144, and 148.

6. The peptide of claim 2, wherein said peptide is selected from SEQ ID NO: 3, 10, 11, 26, 42, 48, 56, 64, 92, 107, 109, 114, 125, 132, and 141.

7. The peptide of claim 2, wherein said peptide is selected from SEQ ID NO: 4, 14, 15, 24, 28, 29, 30, 31, 32, 41, 71, 73, 88, 101, 115, 122, 127, and 135.

8. The peptide of claim 2, wherein said peptide is selected from SEQ ID NO: 5, 6, 12, 18, 51, 55, 58, 63, 68, 75, 81, 85, 93, 97, 116, 117, 131, 138, and 145.

9. The peptide of claim 1, wherein said peptide is PEGylated.

10. The peptide of claim 9, wherein said peptide is PEGylated at the C-terminus.

11. The peptide of claim 9, wherein PEG is selected from

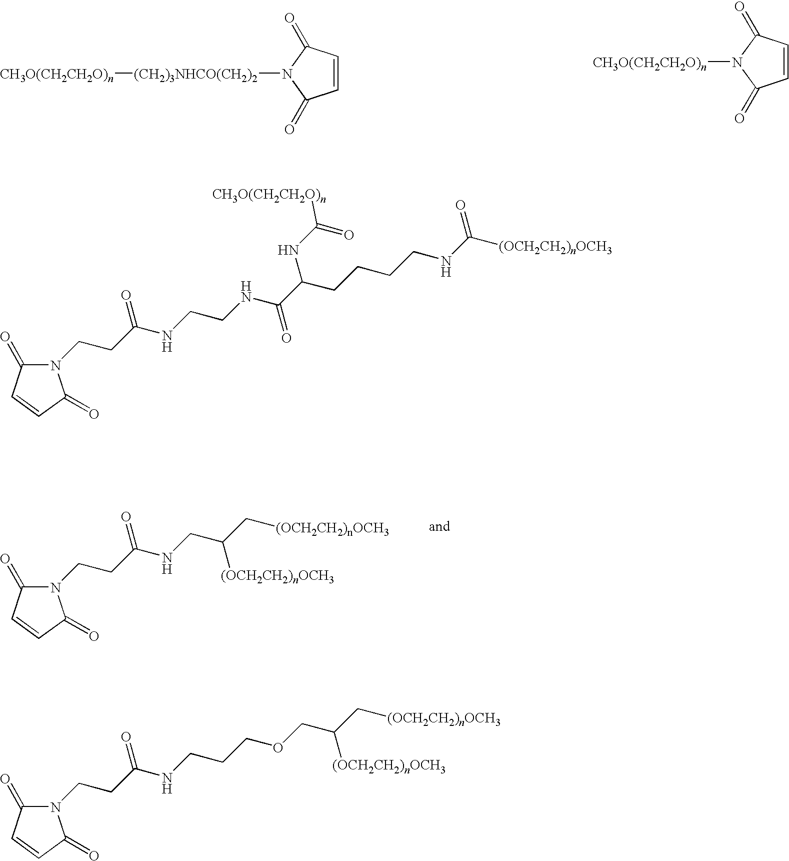

12. The peptide of claim 1, wherein said peptide is acetylated.

13. The peptide of claim 1, wherein Z1 is selected from

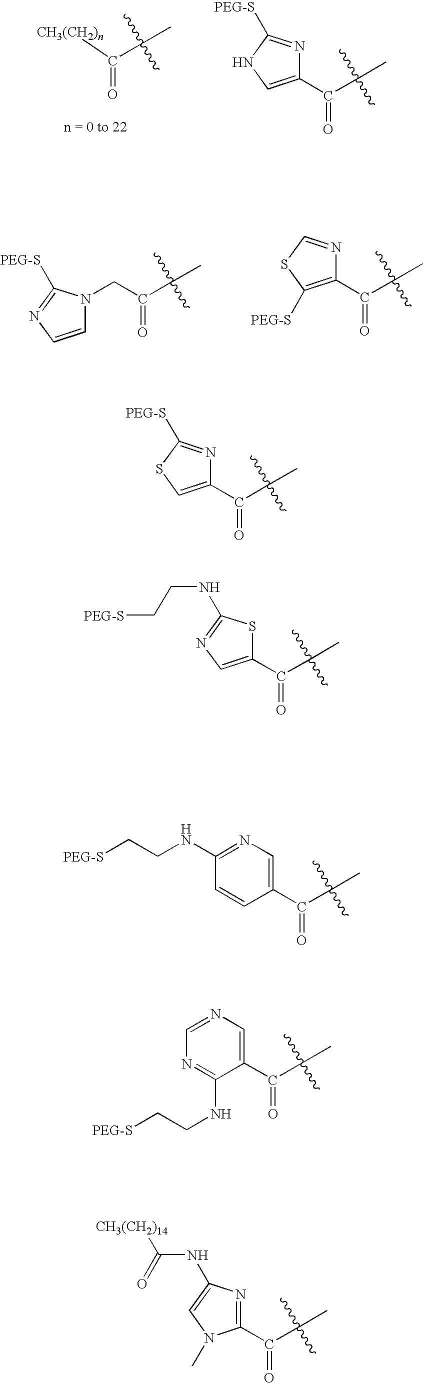

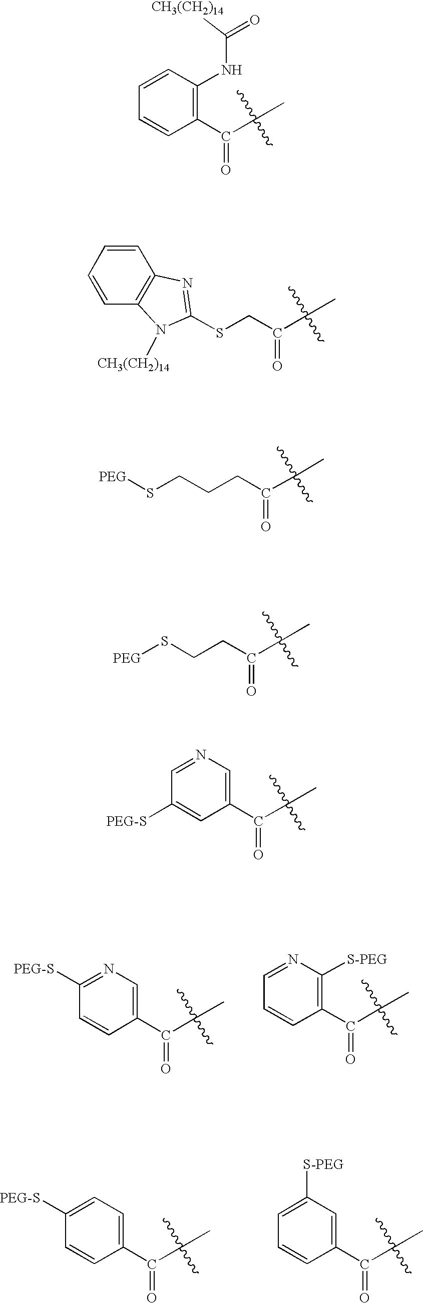

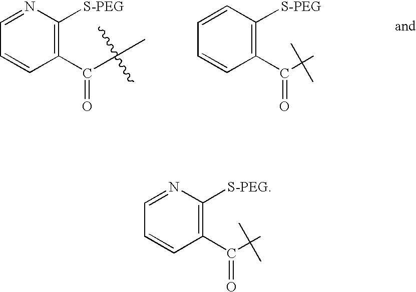

14. The peptide of claim 1, wherein Z1 is selected from

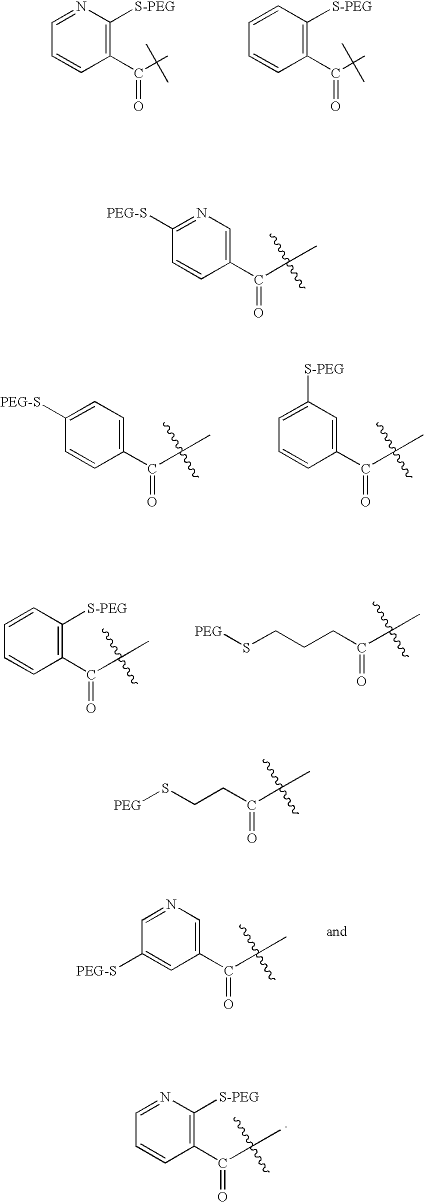

15. The peptide of claim 13, wherein PEG is selected from

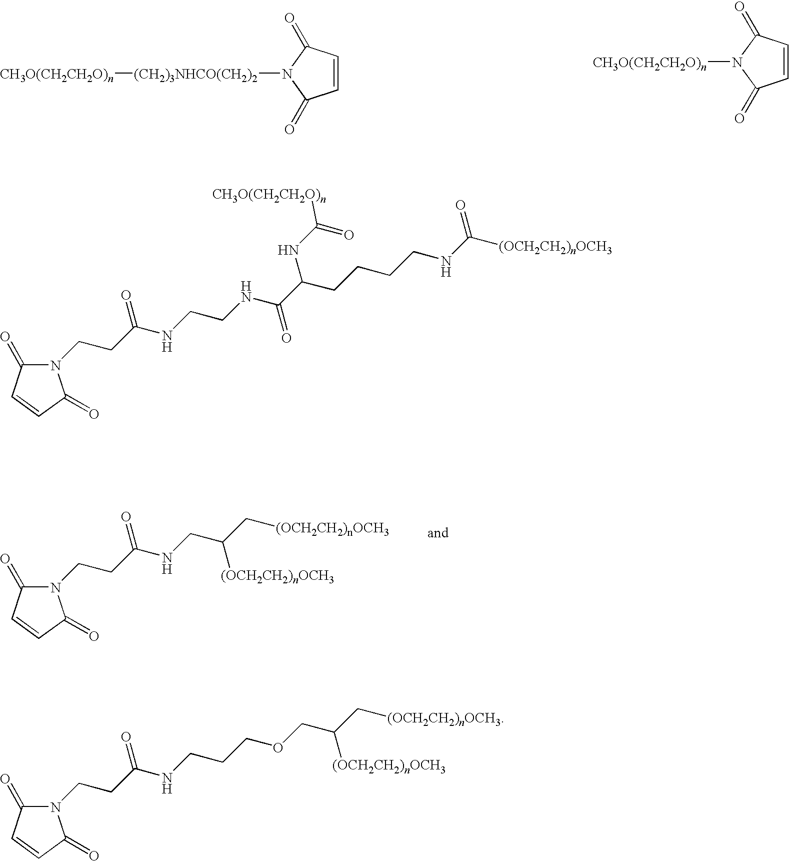

16. A polynucleotide encoding a peptide of SEQ ID NO: 1-156, or a degenerate variant thereof.

17. A vector comprising a polynucleotide of claim 16.

18. A host cell comprising a vector of claim 17.

19. A method for producing a peptide comprising:
a) culturing the host cell of claim 18 under conditions suitable for the expression of said polypeptide; and
b) recovering the peptide from the host cell culture.

20. A purified antibody which binds specifically to the peptide of claim 1.

21. A pharmaceutical composition comprising an effective amount of a peptide of claim 1, in combination with a pharmaceutically acceptable carrier.

22. A pharmaceutical composition comprising a therapeutically effective amount of claim 1, in combination with a pharmaceutically acceptable carrier and one or more pharmaceutical agents.

23. The pharmaceutical composition of claim 22, wherein said pharmaceutical agent is selected from the group consisting of PPAR ligands, insulin secretagogues, sulfonylurea drugs, α-glucosidase inhibitors, insulin sensitizers, hepatic glucose output lowering compounds, insulin and insulin derivatives, biguanides, protein tyrosine phosphatase-1B, dipeptidyl peptidase IV, 11beta-HSD inhibitors, anti-obesity drugs, HMG-CoA reductase inhibitors, nicotinic acid, lipid lowering drugs, ACAT inhibitors, bile acid sequestrants, bile acid reuptake inhibitors, microsomal triglyceride transport inhibitors, fibric acid derivatives, β-blockers, ACE inhibitors, calcium channel blockers, diuretics, renin inhibitors, AT-1 receptor antagonists, ET receptor antagonists, neutral endopeptidase inhibitors, vasopepsidase inhibitors, and nitrates.

24. A method of treating a condition comprising the step of administering to a subject in need thereof a therapeutically effective amount of a peptide of claim 1.

25. The method of claim 24, wherein said condition is diabetes Syndrome X, a diabetes-related disorder, secondary cause of diabetes, cardiovascular disorder, or obesity.

26. (canceled)

27. (canceled)

28. (canceled)

29. (canceled)

30. (canceled)

31. The method of claim 24, further comprising the step of administering the said peptide in combination with one or more pharmaceutical agents.

32. The method of claim 31, wherein said pharmaceutical agent is selected from the group consisting of PPAR agonists, sulfonylurea drugs, non-sulfonylurea secretagogues, α-glucosidase inhibitors, insulin sensitizers, insulin secretagogues, hepatic glucose output lowering compounds, insulin, and anti-obesity agents.

33. The method of claim 24, wherein said diabetes is selected from the group consisting of type 1 diabetes, type 2 diabetes, maturity-onset diabetes of the young, latent autoimmune diabetes adult, and gestational diabetes.

34. (canceled)

35. (canceled)

36. (canceled)

37. The method of claim 24, wherein said diabetes-related disorder is selected from the group consisting of hyperglycemia, hyperinsulinemia, impaired glucose tolerance, impaired fasting glucose, dyslipidemia, hypertriglyceridemia, and insulin resistance.

38. (canceled)

39. (canceled)

40. (canceled)

41. A method of treating diabetes, Syndrome X, diabetes-related disorders or secondary causes of diabetes comprising the step of administering to a subject in need thereof a therapeutically effective amount of a peptide of any one of claims 1-15 in combination with one or more agents selected from the group consisting of HMG-CoA reductase inhibitors, nicotinic acid, lipid lowering drugs, ACAT inhibitors, bile acid sequestrants, bile acid reuptake inhibitors, microsomal triglyceride transport inhibitors, fibric acid derivatives, β-blockers, ACE inhibitors, calcium channel blockers, diuretics, renin inhibitors, AT-1 receptor antagonists, ET receptor antagonists, neutral endopeptidase inhibitors, vasopepsidase inhibitors, and nitrates.

42. The method of claim 41, wherein said diabetes-related disorder is selected from the group consisting of hyperglycemia, hyperinsulinemia, impaired glucose tolerance, impaired fasting glucose, dyslipidemia, hypertriglyceridemia, and insulin resistance.

43. The method of claim 31, wherein the peptide and the one or more pharmaceutical agents are administered as a single pharmaceutical dosage formulation.

44. (canceled)

45. The method of claim 24, wherein said cardiovascular disease is selected from atherosclerosis, coronary heart disease, coronary artery disease, and hypertension.

46. (canceled)

47. A method of stimulating insulin secretion in a subject in need thereof by administering to said subject a therapeutically effective amount of a peptide of claim 1.

48. (canceled)

49. (canceled)

50. (canceled)

51. (canceled)
